(19) United States
(12) Patent Application Publication
(10) Pub. No.: US 2011/0165170 A1
(43) Pub. Date: Jul. 7, 2011

(54) OSTEOPONTIN ANTIBODIES

(75) Inventors: Trisha Ann Haubrich, San Diego, CA (US); Justin Thomas Bingham, Coronado, CA (US); Jitesh Pranial Jani, San Diego, CA (US); Alessandra Blasina, San Diego, CA (US); Kathrin Ladetzki-Baehs, Planegg (DE); Michael Tesar, Planegg-Martinsried (DE); Justin Guy Chapman, San Diego, CA (US); Dirk Ponsel, Germering (DE); Michael A. North, Rancho Santa Fe, CA (US)

(73) Assignee: Pfizer Inc., New York, NY (US)

(21) Appl. No.: 12/859,725

(22) Filed: Aug. 19, 2010

Related U.S. Application Data
(60) Provisional application No. 61/235,542, filed on Aug. 20, 2009.

(51) Int. Cl. A61K 39/395; C07K 16/18; C07H 21/00; C12N 15/63; C12N 1/21; C12N 5/10; C12P 21/02; A61P 35/00; A61P 35/04

(52) U.S. Cl. 424/158.1; 530/389.2; 530/387.3; 536/23.53; 435/320.1; 435/252.3; 435/325; 435/69.6

(57) ABSTRACT

The present disclosure provides isolated antibodies, particularly human antibodies, or antigen binding portions thereof, that bind to osteopontin with high affinity. Nucleic acid molecules encoding the antibodies of the disclosure, expression vectors, host cells and methods for expressing the antibodies of the disclosure are also provided.

Immunoconjugates, bispecific molecules and pharmaceutical compositions comprising the antibodies or antigen binding portions thereof are also provided. The disclosure also provides methods for treating various cancers using the anti-osteopontin antibodies or antigen binding portions thereof described herein.

### RELATED APPLICATIONS

This application claims the benefit of U.S. Patent Application No. 61/235,542, filed Aug. 20, 2009, which is hereby incorporated by reference in its entirety.

### REFERENCE TO SEQUENCE LISTING

This application is being filed electronically via EFS-Web and includes an electronically submitted sequence listing in .txt format. The .txt file contains a sequence listing entitled “PC33873A_SequenceListing.txt” created on Aug. 19, 2010 and having a size of 81 KB. The sequence listing contained in this .txt file is part of the specification and is incorporated herein by reference in its entirety.

### FIELD

The present disclosure relates to antibodies and antigen-binding portions thereof that bind to osteopontin. The disclosure also relates to nucleic acid molecules encoding such antibodies and antigen-binding portions, methods of making osteopontin antibodies and antigen-binding portions, compositions comprising these antibodies and antigen-binding portions, and methods of using the antibodies, antigen-binding portions, and compositions.

### BACKGROUND

The human osteopontin (also known as SPP1) gene encodes a 314 amino acid residue precursor protein with a 16 amino acid residue predicted signal peptide that is cleaved to yield a 298 amino acid residue mature protein with an integrin binding sequence and N- and O-glycosylation sites. Osteopontin (OPN) is a secreted glycosylated phosphoprotein with a molecular weight between 44 and 75 kDa depending on posttranslational modifications of phosphorylation and/ or sulphation (Sodek et al., Crit. Rev. Oral Biol. Med. 11(3):279-303 (2000)). OPN contains the classic RGD motif that is known to play a key role in cell attachment. The role of OPN in bone is well-known in the art. Osteoclasts, which are the predominant bone resorbing cell type, express the integrin αvβ3, a membrane-associated receptor for OPN (Dodds et al., J. Bone Miner. Res. 10(11):1666-1680 (1995)). OPN is capable of binding to several cell types including osteoblasts, osteoclasts, non transformed calvaria cell lines and many transformed fibroblast cell lines (Somerman et al., Matrix 9(1):49-54 (1989)). It has also been reported that OPN associates with fibronectin (Singh et al., J. Biol. Chem. 265(30):18696-18701 (1990); Nemir et al., J. Biol. Chem. 264(30):18202-18208 (1989)), type I collagen (Chen et al., J. Biol. Chem. 267(34):24871-24878 (1992)), and osteocalcin (Ritter et al., J. Bone Miner. Res. 7(8):877-885 (1992)).

Aside from cell attachment, OPN can also affect cell physiology by interaction with its receptor in a calcium dependent manner. OPN is capable of binding multiple Ca²⁺ ions with relatively low affinity, and the conformation of OPN is highly sensitive to changes in the concentration of free Ca²⁺. The high density of negative charges around the RGD cell binding sequence suggests that folding of the protein in that region is dependent on free calcium levels, suggesting that calcium may affect the interaction of OPN with integrins.

OPN expression is also known to play a major role in malignant carcinomas. Initially it was suggested that macrophages infiltrating the tumor, rather than the tumor cells themselves, express OPN (Furger et al., Curr. Mol. Med. 1(5):621-632 (2001)). More recently, however, certain tumor cells have been reported to directly express OPN (Rittling et al., Br. J. Cancer 90(10):1877-1881 (2004)). Elevated levels of OPN were detected in malignant breast tumors (Bellahcene et al., Am. J. Pathol. 146(1):95-100 (1995)), malignant glioblastomas (Takano et al., Br. J. Cancer 82(12):1967-1973 (2000)), invasive primary cutaneous melanoma (Zhou et al., J. Invest. Dermatol. 124(5):1044-1052 (2005)) and ovarian cancer (Brakora et al., Gynecol. Oncol. 93(2):361-365 (2004)) and strongly correlate with poor patient survival (Bramwell et al., Clin. Cancer Res. 12(11):3337-3343 (2006).

### SUMMARY

It is an object of the disclosure to provide human, or humanized antibodies that specifically bind osteopontin. It is another object of the disclosure to provide antibodies that are safe for human administration. It is also an object of the present disclosure to provide methods for treating disease and/or conditions associated with osteopontin up-regulation by using one or more antibodies of the disclosure. These and other objects of the disclosure are more fully described herein.

In one aspect, the disclosure provides an isolated human antibody or antigen-binding portion thereof that specifically binds osteopontin with a K_{D} of 600 nM or less, 100 nM or less, 50 nM or less, 10 nM or less, 5 nM or less, or 1 nM or less. In one aspect, said osteopontin is human osteopontin. In another aspect, said osteopontin is murine osteopontin.

In a further aspect, the disclosure provides an isolated antibody, or antigen-binding portion thereof, that specifically binds osteopontin, wherein said antibody or antigen-binding portion comprises: (a) an H-CDR1 as set forth in SEQ ID NO:1, SEQ ID NO:15, or SEQ ID NO:29; (b) an H-CDR2 as set forth in SEQ ID NO:2, SEQ ID NO:16, or SEQ ID NO:30; and (c) an H-CDR3 as set forth in SEQ ID NO:3, SEQ ID NO: 17, or SEQ ID NO:31. In a further aspect, such antibodies or antigen-binding portions further comprise: (a) an L-CDR1 as set forth in SEQ ID NO:4, SEQ ID NO:18, or SEQ ID NO:32; (b) an L-CDR2 as set forth in SEQ ID NO:5, SEQ ID NO:19, or SEQ ID NO:33; and (c) an L-CDR3 as set forth in SEQ ID NO:6, SEQ ID NO:20, SEQ ID NO:34, or SEQ ID NO:75.

In a further aspect, the disclosure provides an isolated antibody, or antigen-binding portion thereof, that specifically binds osteopontin, wherein said antibody or antigen-binding portion comprises: (a) an L-CDR1 as set forth in SEQ ID NO:4, SEQ ID NO:18, or SEQ ID NO:32; (b) an L-CDR2 as set forth in SEQ ID NO:5, SEQ ID NO:19, or SEQ ID NO:33; and (c) an L-CDR3 as set forth in SEQ ID NO:6, SEQ ID NO:20, SEQ ID NO:34, or SEQ ID NO:75. In a further aspect, such antibodies or antigen-binding portions further comprise: (a) an H-CDR1 as set forth in SEQ ID NO:1, SEQ ID NO:15, or SEQ ID NO:29; (b) an H-CDR2 as set forth in SEQ ID NO:2, SEQ ID NO:16, or SEQ ID NO:30; and (c) an H-CDR3 as set forth in SEQ ID NO:3, SEQ ID NO:17, or SEQ ID NO:31.

In a further aspect, the disclosure provides an isolated antibody, or antigen-binding portion thereof, that specifically binds osteopontin, wherein said antibody or antigen-binding portion comprises an H-CDR1 as set forth in SEQ ID NO:1, an H-CDR2 as set forth in SEQ ID NO:2, and an H-CDR3 as set forth in SEQ ID NO:3.

In a further aspect, the disclosure provides an isolated antibody, or antigen-binding portion thereof, that specifically binds osteopontin, wherein said antibody or antigen-binding portion comprises an H-CDR1 as set forth in SEQ ID NO:15, an H-CDR2 as set forth in SEQ ID NO:16, and an H-CDR3 as set forth in SEQ ID NO:17.

In a further aspect, the disclosure provides an isolated antibody, or antigen-binding portion thereof, that specifically binds osteopontin, wherein said antibody or antigen-binding portion comprises an H-CDR1 as set forth in SEQ ID NO:29, an H-CDR2 as set forth in SEQ ID NO:30, and an H-CDR3 as set forth in SEQ ID NO:31.

In a further aspect, the disclosure provides an isolated antibody, or antigen-binding portion thereof, that specifically binds osteopontin, wherein said antibody or antigen-binding portion comprises an L-CDR1 as set forth in SEQ ID NO:4, an L-CDR2 as set forth in SEQ ID NO:5, and an L-CDR3 as set forth in SEQ ID NO:6.

In a further aspect, the disclosure provides an isolated antibody, or antigen-binding portion thereof, that specifically binds osteopontin, wherein said antibody or antigen-binding portion comprises an L-CDR1 as set forth in SEQ ID NO:18, an L-CDR2 as set forth in SEQ ID NO:19, and an L-CDR3 as set forth in SEQ ID NO:20.

In a further aspect, the disclosure provides an isolated antibody, or antigen-binding portion thereof, that specifically binds osteopontin, wherein said antibody or antigen-binding portion comprises an L-CDR1 as set forth in SEQ ID NO:32, an L-CDR2 as set forth in SEQ ID NO:33, and an L-CDR3 as set forth in SEQ ID NO:34.

In a further aspect, the disclosure provides an isolated antibody, or antigen-binding portion thereof, that specifically binds osteopontin, wherein said antibody or antigen-binding portion comprises an L-CDR1 as set forth in SEQ ID NO:4, an L-CDR2 as set forth in SEQ ID NO:5, and an L-CDR3 as set forth in SEQ ID NO:75.

In a further aspect, the disclosure provides an isolated antibody, or antigen-binding portion thereof, that specifically binds osteopontin, wherein said antibody or antigen-binding portion comprises an H-CDR1 as set forth in SEQ ID NO:1, an H-CDR2 as set forth in SEQ ID NO:2, an H-CDR3 as set forth in SEQ ID NO:3, an L-CDR1 as set forth in SEQ ID NO:4, an L-CDR2 as set forth in SEQ ID NO:5, and an L-CDR3 as set forth in SEQ ID NO:6.

In a further aspect, the disclosure provides an isolated antibody, or antigen-binding portion thereof, that specifically binds osteopontin, wherein said antibody or antigen-binding portion comprises an H-CDR1 as set forth in SEQ ID NO:15, an H-CDR2 as set forth in SEQ ID NO:16, an H-CDR3 as set forth in SEQ ID NO:17, an L-CDR1 as set forth in SEQ ID NO:18, an L-CDR2 as set forth in SEQ ID NO:19, and an L-CDR3 as set forth in SEQ ID NO:20.

In a further aspect, the disclosure provides an isolated antibody, or antigen-binding portion thereof, that specifically binds osteopontin, wherein said antibody or antigen-binding portion comprises an H-CDR1 as set forth in SEQ ID NO:29, an H-CDR2 as set forth in SEQ ID NO:30, an H-CDR3 as set forth in SEQ ID NO:31, an L-CDR1 as set forth in SEQ ID NO:32, an L-CDR2 as set forth in SEQ ID NO:33, and an L-CDR3 as set forth in SEQ ID NO:34.

In a further aspect, the disclosure provides an isolated antibody, or antigen-binding portion thereof, that specifically binds osteopontin, wherein said antibody or antigen-binding portion comprises an H-CDR1 as set forth in SEQ ID NO:1, an H-CDR2 as set forth in SEQ ID NO:2, an H-CDR3 as set forth in SEQ ID NO:3, an L-CDR1 as set forth in SEQ ID NO:4, an L-CDR2 as set forth in SEQ ID NO:5, and an L-CDR3 as set forth in SEQ ID NO:75.

In a further aspect, the disclosure provides an isolated antibody, or antigen-binding portion thereof, that specifically binds osteopontin, wherein said antibody or antigen-binding portion comprises a V_{H} chain amino acid sequence as set forth in SEQ ID NO:7, SEQ ID NO:21, SEQ ID NO:35, SEQ ID NO:44, SEQ ID NO:48, or SEQ ID NO:52. In one aspect, said antibody or antigen-binding portion further comprises a V_{L} chain amino acid sequence as set forth in SEQ ID NO:8, SEQ ID NO:22, SEQ ID NO:36, SEQ ID NO:46, SEQ ID NO:50, SEQ ID NO:54, SEQ ID NO:56 or SEQ ID NO:76.

In a further aspect, the disclosure provides an isolated antibody or antigen-binding portion thereof that specifically binds osteopontin, wherein said antibody or antigen-binding portion comprises a V_{L} chain amino acid sequence as set forth in SEQ ID NO:8, SEQ ID NO:22, SEQ ID NO:36, SEQ ID NO:46, SEQ ID NO:50, SEQ ID NO:54, SEQ ID NO:56, or SEQ ID NO:76. In one embodiment, said antibody or antigen-binding portion further comprises a V_{H} chain amino acid sequence as set forth in SEQ ID NO:7, SEQ ID NO:21, SEQ ID NO:35, SEQ ID NO:44, SEQ ID NO:48, or SEQ ID NO:52.

In one aspect, the disclosure provides an isolated antibody, or antigen-binding portion thereof, that specifically binds osteopontin, wherein said antibody or antigen-binding portion comprises a V_{H} chain amino acid sequence as set forth in SEQ ID NO:7 and a V_{L} chain amino acid sequence as set forth in SEQ ID NO:8.

In one aspect, the disclosure provides an isolated antibody, or antigen-binding portion thereof, that specifically binds osteopontin, wherein said antibody or antigen-binding portion comprises a V_{H} chain amino acid sequence as set forth in SEQ ID NO:21 and a V_{L} chain amino acid sequence as set forth in SEQ ID NO:22.

In one aspect, the disclosure provides an isolated antibody, or antigen-binding portion thereof, that specifically binds osteopontin, wherein said antibody or antigen-binding portion comprises a V_{H} chain amino acid sequence as set forth in SEQ ID NO:35 and a V_{L} chain amino acid sequence as set forth in SEQ ID NO:36.

In one aspect, the disclosure provides an isolated antibody, or antigen-binding portion thereof, that specifically binds osteopontin, wherein said antibody or antigen-binding portion comprises a V_{H} chain amino acid sequence as set forth in SEQ ID NO:44 and a V_{L} chain amino acid sequence as set forth in SEQ ID NO:46.

In one aspect, the disclosure provides an isolated antibody, or antigen-binding portion thereof, that specifically binds osteopontin, wherein said antibody or antigen-binding portion comprises a V_{H} chain amino acid sequence as set forth in SEQ ID NO:48 and a V_{L} chain amino acid sequence as set forth in SEQ ID NO:50.

In one aspect, the disclosure provides an isolated antibody, or antigen-binding portion thereof, that specifically binds osteopontin, wherein said antibody or antigen-binding portion comprises a V_{H} chain amino acid sequence as set forth in SEQ ID NO:52 and a V_{L} chain amino acid sequence as set forth in SEQ ID NO:54.

In one aspect, the disclosure provides an isolated antibody, or antigen-binding portion thereof, that specifically binds osteopontin, wherein said antibody or antigen-binding portion comprises a V_{H} chain amino acid sequence as set forth in SEQ ID NO:52 and a V_{L} chain amino acid sequence as set forth in SEQ ID NO:56.

In one aspect, the disclosure provides an isolated antibody, or antigen-binding portion thereof, that specifically binds osteopontin, wherein said antibody or antigen-binding portion comprises a V_{H} chain amino acid sequence as set forth in SEQ ID NO:7 and a V_{L} chain amino acid sequence as set forth in SEQ ID NO:76.

In one aspect, the osteopontin to which any of the antibodies, or antigen-binding portions, described herein specifically bind is human osteopontin. In a further aspect, said osteopontin is murine osteopontin.

In another aspect, the disclosure provides an antibody according to any of the antibodies as described herein, which is an IgG. For example, said antibodies can be IgG1, IgG2, IgG3, or IgG4.

In a further aspect, the disclosure provides an antibody according to any of the antibodies as described herein, which is a human, humanized, or chimeric antibody.

In a further aspect, the disclosure provides an antigen-binding portion according to any of the antigen-binding portions described herein, which is a Fab or scFv antibody fragment.

In one aspect, the C-terminal lysine of the heavy chain of any of the anti-OPN antibodies of the disclosure as described is cleaved, and is thus not present. For example, in a further aspect, the disclosure provides an isolated antibody comprising a heavy chain amino acid sequence as set forth in SEQ ID NO:11; and a light chain amino acid sequence as set forth in SEQ ID NO:12, with the proviso that the C-terminal lysine residue of SEQ ID NO:11 is optionally not present.

In a further aspect, the disclosure provides an isolated antibody comprising a heavy chain amino acid sequence as set forth in SEQ ID NO:25; and a light chain amino acid sequence as set forth in SEQ ID NO:26, with the proviso that the C-terminal lysine residue of SEQ ID NO:25 is optionally not present.

In a further aspect, the disclosure provides an isolated antibody comprising a heavy chain amino acid sequence as set forth in SEQ ID NO:39; and a light chain amino acid sequence as set forth in SEQ ID NO:40, with the proviso that the C-terminal lysine residue of SEQ ID NO:39 is optionally not present.

In a further aspect, the disclosure provides an isolated antibody comprising a heavy chain amino acid sequence as set forth in SEQ ID NO:58; and a light chain amino acid sequence as set forth in SEQ ID NO:59, with the proviso that the C-terminal lysine residue of SEQ ID NO:58 is optionally not present.

In a further aspect, the disclosure provides an isolated antibody comprising a heavy chain amino acid sequence as set forth in SEQ ID NO:62; and a light chain amino acid sequence as set forth in SEQ ID NO:63, with the proviso that the C-terminal lysine residue of SEQ ID NO:62 is optionally not present.

In a further aspect, the disclosure provides an isolated antibody comprising a heavy chain amino acid sequence as set forth in SEQ ID NO:66; and a light chain amino acid sequence as set forth in SEQ ID NO:67, with the proviso that the C-terminal lysine residue of SEQ ID NO:66 is optionally not present.

In a further aspect, the disclosure provides an isolated antibody comprising a heavy chain amino acid sequence as set forth in SEQ ID NO:11; and a light chain amino acid sequence as set forth in SEQ ID NO:78, with the proviso that the C-terminal lysine residue of SEQ ID NO:11 is optionally not present.

In a still further aspect, the disclosure provides an isolated antibody or antigen-binding portion thereof comprising a V_{H} chain that is encoded by (i) a nucleic acid sequence comprising SEQ ID NO:9, SEQ ID NO:23, SEQ ID NO:37, SEQ ID NO:45, SEQ ID NO:49, or SEQ ID NO:53, or (ii) a nucleic acid sequences that hybridizes under high stringency conditions to the complementary strand of SEQ ID NO:9, SEQ ID NO:23, SEQ ID NO:37, SEQ ID NO:45, SEQ ID NO:49, or SEQ ID NO:53, wherein said antibody or antigen-binding portion specifically binds osteopontin.

In a further aspect, the disclosure provides an isolated antibody, or antigen-binding portion thereof, that competes, and/or that cross-competes for binding to OPN with any of the OPN antibodies or antigen-binding portions disclosed herein. For example, an antibody, or antigen binding portion thereof that specifically binds to OPN and that competes for binding to OPN, and/or that cross-competes for binding to OPN with a monoclonal antibody selected from 6990, 6991, and 6993. In one aspect, such isolated antibody is a human antibody. In another aspect, such isolated antibody is a humanized antibody.

In a further aspect there is provided an isolated antibody or antigen binding portion thereof that binds to the same epitope on human OPN as any of the antibodies disclosed herein and/or competes for binding to human OPN with such an antibody. For example, an antibody, or antigen binding portion thereof, that specifically binds to OPN, and that binds to the same epitope on human OPN as a monoclonal antibody selected from 6990, 6991, and 6993, and/or competes for binding to human OPN with a monoclonal antibody selected from 6990, 6991, and 6993.

A further aspect of the present disclosure is an isolated antibody, or an antigen-binding portion thereof, comprising a heavy chain variable region that is the product of, or derived from, a human V_{H} 3-23 gene, wherein the antibody specifically binds OPN.

A further aspect of the present disclosure is an isolated monoclonal antibody, or an antigen-binding portion thereof, comprising a light chain variable region that is the product of, or derived from, a human V_{L} λ3 or λ1-13 gene, wherein the antibody specifically binds OPN.

In a further aspect, the disclosure provides an immunoconjugate comprising any of the antibodies, or antigen-binding portions thereof, as described herein, linked to a therapeutic agent. In one case, the therapeutic agent is a cytotoxin or a radioactive isotope. In a further aspect, the disclosure provides a composition comprising any of the immunoconjugates described herein and a pharmaceutically acceptable carrier. The disclosure also provides a bispecific molecule comprising an antibody, or antigen-binding portion thereof, linked to a second functional moiety having a different binding specificity than said antibody, or antigen binding portion thereof.

In a further aspect, the disclosure provides a method for preparing an anti-OPN antibody comprising:

(a) providing: (i) a heavy chain variable region antibody sequence comprising a H-CDR1 sequence selected from the group consisting of SEQ ID NOs: 1, 15, and 29, a H-CDR2 sequence selected from the group consisting of SEQ ID NOs: 2, 16, and 30, and/or a H-CDR3 sequence selected from the group consisting of SEQ ID NOs: 3, 17, and 31; and/or (ii) a light chain variable region antibody sequence comprising a L-CDR1 sequence selected from the group consisting of SEQ ID NOs: 4, 18, and 32, a L-CDR2 sequence selected from the group consisting of SEQ ID NOs: 5, 19, and 33, and/or a L-CDR3 sequence selected from the group consisting of SEQ ID NOs: 6, 20, 34, and 75; and

(b) expressing the antibody sequence as a protein.

In a further aspect, the disclosure provides a method for preparing an anti-OPN antibody comprising:

(a) providing: (i) a nucleic acid sequence that encodes a heavy chain variable region antibody sequence comprising a H-CDR1 sequence selected from the group consisting of SEQ ID NOs: 1, 15, and 29, a H-CDR2 sequence selected from the group consisting of SEQ ID NOs: 2, 16, and 30, and/or a H-CDR3 sequence selected from the group consisting of SEQ ID NOs: 3, 17, and 31; and/or (ii) a nucleic acid sequence that that encodes a light chain variable region antibody sequence comprising a L-CDR1 sequence selected from the group consisting of SEQ ID NOs: 4, 18, and 32, a L-CDR2 sequence selected from the group consisting of SEQ ID NOs: 5, 19, and 33, and/or a L-CDR3 sequence selected from the group consisting of SEQ ID NOs: 6, 20, 34, and 75; and

(b) expressing the nucleic acid sequence to produce an antibody or an antigen-binding portion thereof.

In a further aspect, the disclosure provides a method for preparing an anti-OPN antibody comprising:

(a) providing: (i) a heavy chain variable region antibody sequence comprising a H-CDR1 sequence selected from the group consisting of SEQ ID NOs: 1, 15, and 29, a H-CDR2 sequence selected from the group consisting of SEQ ID NOs: 2, 16, and 30, and/or a H-CDR3 sequence selected from the group consisting of SEQ ID NOs: 3, 17, and 31; and/or (ii) a light chain variable region antibody sequence comprising a L-CDR1 sequence selected from the group consisting of SEQ ID NOs: 4, 18, and 32, a L-CDR2 sequence selected from the group consisting of SEQ ID NOs: 5, 19, and 33, and/or a L-CDR3 sequence selected from the group consisting of SEQ ID NOs: 6, 20, 34, and 75;

(b) altering at least one amino acid residue within the heavy chain variable region antibody sequence and/or the light chain variable region antibody sequence to create at least one altered antibody sequence; and

(c) expressing the altered antibody sequence as a protein.

In a still further aspect, the disclosure provides an isolated antibody or antigen-binding portion thereof comprising a V_{L} chain that is encoded by (i) a nucleic acid sequence comprising SEQ ID NO:10, SEQ ID NO:24, SEQ ID NO:38, SEQ ID NO:47, SEQ ID NO:51, SEQ ID NO:55, SEQ ID NO:57, or SEQ ID NO:77, or (ii) a nucleic acid sequences that hybridizes under high stringency conditions to the complementary strand of SEQ ID NO:10, SEQ ID NO:24, SEQ ID NO:38, SEQ ID NO:47, SEQ ID NO:51, SEQ ID NO:55, SEQ ID NO:57, or SEQ ID NO:77, wherein said antibody or antigen-binding portion specifically binds OPN.

In a further aspect, the disclosure provides an isolated nucleic acid that encodes any of the antibodies as described herein.

In a further aspect, the disclosure provides an isolated nucleic acid that encodes a V_{H} chain of an antibody or antigen-binding portion thereof, and that comprises (i) SEQ ID NO:9, SEQ ID NO:23, SEQ ID NO:37, SEQ ID NO:45, SEQ ID NO:49, or SEQ ID NO:53; or (ii) a nucleic acid sequence that hybridizes under high stringency conditions to the complementary strand of SEQ ID NO:9, SEQ ID NO:23, SEQ ID NO:37, SEQ ID NO:45, SEQ ID NO:49, or SEQ ID NO:53; wherein said antibody or antigen-binding portion specifically binds OPN.

In a still further aspect is provided an isolated nucleic acid that encodes a V_{L} chain of an antibody or antigen-binding portion thereof, and that comprises (i) SEQ ID NO:10, SEQ ID NO:24, SEQ ID NO:38, SEQ ID NO:47, SEQ ID NO:51, SEQ ID NO:55, SEQ ID NO:57, or SEQ ID NO:77; or (ii) a nucleic acid sequence that hybridizes under high stringency conditions to the complementary strand of SEQ ID NO:10, SEQ ID NO:24, SEQ ID NO:38, SEQ ID NO:47, SEQ ID NO:51, SEQ ID NO:55, SEQ ID NO:57, or SEQ ID NO:77; wherein said antibody or antigen-binding portion specifically binds OPN.

In a further aspect, the disclosure provides a vector comprising any of the nucleic acids described herein. In a still further aspect, the disclosure provides a host cell comprising any of the vectors described herein. For example, such host cells can be bacterial or mammalian.

In a further aspect, the disclosure provides a pharmaceutical composition comprising any of the antibodies or antigen-binding portions, immunoconjugates, or bispecific molecules described herein and a pharmaceutically acceptable carrier or excipient.

The disclosure further provides methods for treating abnormal cell growth comprising administering to a subject in need thereof an effective amount of any of the pharmaceutical compositions described herein. The disclosure further provides methods of reducing tumor cell metastasis in a subject, comprising administering to said subject an effective amount of any of the antibodies, antigen-binding portions, or pharmaceutical compositions described herein.

In a further aspect, the disclosure provides a use of any of the antibodies, antigen-binding portions, or pharmaceutical compositions described herein, for the manufacture of a medicament for the treatment of abnormal cell growth in a subject in need thereof. In a still further aspect, the disclosure provides a use of any of the antibodies, antigen-binding portions, or pharmaceutical compositions described herein, for the manufacture of a medicament for the treatment of tumor cell metastasis in a subject in need thereof.

In a further aspect, the disclosure provides the antibodies, antigen-binding portions, or pharmaceutical compositions described herein for use in the treatment of abnormal cell growth in a subject in need thereof. In a still further aspect, the disclosure provides the antibodies, antigen-binding portions, or pharmaceutical compositions described herein for use in the treatment of tumor cell metastasis in a subject in need thereof.

In another aspect, the disclosure provides methods of preparing an anti-ostepontin antibody, or antigen-binding portion thereof, comprising expressing the antibody or antigen-binding portion in any of the host cells described herein.

In a further aspect, the disclosure provides any of the human antibodies, or antigen-binding portions thereof, wherein said human antibodies or antigen-binding portions are synthetic human antibodies or antigen-binding portions.

The present disclosure further provides antibodies, or antigen-binding portions thereof, comprising peptide variants of any of the specific sequences disclosed herein (e.g. SEQ ID NOs: 1 to 42, and 44 to 69). Such peptide variants can include both conservative and non-conservative substitutions, deletions, and/or additions, and typically include peptides that are at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% identical to any of the specific sequences disclosed herein.

For example, in one aspect, the disclosure provides an isolated antibody or antigen-binding portion thereof that comprises a V_{H} chain amino acid sequence as set forth in SEQ ID NO:7, SEQ ID NO:21, SEQ ID NO:35, SEQ ID NO:44, SEQ ID NO:48, or SEQ ID NO:52 or a peptide variant thereof. In one aspect, said peptide variant comprises 1, 2, 3, 4, 5, 6, 7, 8, 9, 10, 11, 12, 13, 14, or 15 conservative or non-conservative substitutions, and/or 1, 2, 3, 4, 5, 6, 7, 8, 9, 10, 11, 12, 13, 14, or 15 additions and/or deletions to SEQ ID NO:7, SEQ ID NO:21, SEQ ID NO:35, SEQ ID NO:44, SEQ ID NO:48, or SEQ ID NO:52. In a further aspect, said peptide variant is at least 65%, at least 75%, at least 85%, at least 90%, at least 95%, at least 96%, at least 97%, at least 98%, or at least 99% identical to SEQ ID NO:7, SEQ ID NO:21, SEQ ID NO:35, SEQ ID NO:44, SEQ ID NO:48, or SEQ ID NO:52, and wherein said antibody or antigen-binding portion specifically binds osteopontin.

In a further aspect, the disclosure provides an isolated antibody or antigen-binding portion thereof that comprises a V_{L} chain amino acid sequence as set forth in SEQ ID NO:8, SEQ ID NO:22, SEQ ID NO:36, SEQ ID NO:46, SEQ ID NO:50, SEQ ID NO:54, SEQ ID NO:56, or SEQ ID NO:76 or a peptide variant thereof. In one aspect, said peptide variant comprises 1, 2, 3, 4, 5, 6, 7, 8, 9, 10, 11, 12, 13, 14, or 15 conservative or non-conservative substitutions, and/or 1, 2, 3, 4, 5, 6, 7, 8, 9, 10, 11, 12, 13, 14, or 15 additions and/or deletions to SEQ ID NO:8, SEQ ID NO:22, SEQ ID NO:36, SEQ ID NO:46, SEQ ID NO:50, SEQ ID NO:54, SEQ ID NO:56, or SEQ ID NO:76. In a further aspect, said peptide variant is at least 65%, at least 75%, at least 85%, at least 90%, at least 95%, at least 96%, at least 97%, at least 98%, or at least 99% identical to SEQ ID NO:8, SEQ ID NO:22, SEQ ID NO:36, SEQ ID NO:46, SEQ ID NO:50, SEQ ID NO:54, SEQ ID NO:56, or SEQ ID NO:76, and wherein said antibody or antigen-binding portion specifically binds OPN.

### BRIEF DESCRIPTION OF THE FIGURES

FIG. 1A shows the DNA sequence of the MOR-6990 heavy chain variable region—corresponding CDR regions are underlined (SEQ ID NO:9);

FIG. 1B shows the amino acid sequence of the MOR-6990 heavy chain variable region (SEQ ID NO:7)—CDR regions are underlined;

FIG. 1C shows the DNA sequence of the MOR-6990 light chain variable region—corresponding CDR regions are underlined (SEQ ID NO:10);

FIG. 1D shows the amino acid sequence of the MOR-6990 light chain variable region (SEQ ID NO:8)—CDR regions are underlined;

FIG. 1E shows the DNA sequence of the MOR-6991 heavy chain variable region—corresponding CDR regions are underlined (SEQ ID NO:23);

FIG. 1F shows the amino acid sequence of the MOR-6991 heavy chain variable region (SEQ ID NO:21)—CDR regions are underlined;

FIG. 1G shows the DNA sequence of the MOR-6991 light chain variable region—corresponding CDR regions are underlined (SEQ ID NO:24);

FIG. 1H shows the amino acid sequence of the MOR-6991 light chain variable region (SEQ ID NO:20)—CDR regions are underlined;

FIG. 1I shows the DNA sequence of the MOR-6993 heavy chain variable region—corresponding CDR regions are underlined (SEQ ID NO:37);

FIG. 1J shows the amino acid sequence of the MOR-6993 heavy chain variable region (SEQ ID NO:35)—CDR regions are underlined;

FIG. 1K shows the DNA sequence of the MOR-6993 light chain variable region—corresponding CDR regions are underlined (SEQ ID NO:38);

FIG. 1L shows the amino acid sequence of the MOR-6993 light chain variable region (SEQ ID NO:36)—CDR regions are underlined;

FIG. 1M shows the DNA sequence of the MOR-6990-GL heavy chain variable region, where germ line mutations are shown by boxing, and corresponding CDR regions are underlined (SEQ ID NO:45);

FIG. 1N shows the amino acid sequence of the MOR-6990-GL heavy chain variable region, where germ line mutations are shown by boxing, and corresponding CDR regions are underlined (SEQ ID NO:44);

FIG. 1O shows the DNA sequence of the MOR-6990-GL light chain variable region, where germ line mutations are shown by boxing, and corresponding CDR regions are underlined (SEQ ID NO:47);

FIG. 1P shows the amino acid sequence of the MOR-6990-GL light chain variable region, where germ line mutations are shown by boxing, and corresponding CDR regions are underlined (SEQ ID NO:46);

FIG. 1Q shows the DNA sequence of the MOR-6991-GL heavy chain variable region, where germ line mutations are shown by boxing, and corresponding CDR regions are underlined (SEQ ID NO:49);

FIG. 1R shows the amino acid sequence of the MOR-6991-GL heavy chain variable region, where germ line mutations are shown by boxing, and corresponding CDR regions are underlined (SEQ ID NO:48);

FIG. 1S shows the DNA sequence of the MOR-6991-GL light chain variable region, where germ line mutations are shown by boxing, and corresponding CDR regions are underlined (SEQ ID NO:51);

FIG. 1T shows the amino acid sequence of the MOR-6991-GL light chain variable region, where germ line mutations are shown by boxing, and corresponding CDR regions are underlined (SEQ ID NO:50);

FIG. 1U shows the DNA sequence of the MOR-6993-GL heavy chain variable region, where germ line mutations are shown by boxing, and corresponding CDR regions are underlined (SEQ ID NO:53);

FIG. 1V shows the amino acid sequence of the MOR-6993-GL heavy chain variable region, where germ line mutations are shown by boxing, and corresponding CDR regions are underlined (SEQ ID NO:52);

FIG. 1W shows the DNA sequence of the MOR-6993-GL light chain variable region, where germ line mutations are shown by boxing, and corresponding CDR regions are underlined (SEQ ID NO:55);

FIG. 1X shows the amino acid sequence of the MOR-6993-GL light chain variable region, where germ line mutations are shown by boxing, and corresponding CDR regions are underlined (SEQ ID NO:54);

FIG. 1Y shows the DNA sequence of the MOR-6993-GL-V44K light chain variable region, where germ line mutations are shown by boxing, the V44K mutation is shown in bold, and corresponding CDR regions are underlined (SEQ ID NO:57);

FIG. 1Z shows the amino acid sequence of the MOR-6993-GL-V44K light chain variable region, where germ line mutations are shown by boxing, the V44K is shown in bold, and corresponding CDR regions are underlined (SEQ ID NO:56);

FIG. 2A shows the DNA sequence of the MOR-6990 heavy chain (SEQ ID NO:13), where variable region sequence is shown in uppercase, while constant region sequence is shown in lowercase, and with corresponding CDR regions underlined;

FIG. 2B shows the amino acid sequence of the MOR-6990 heavy chain (SEQ ID NO:11), where variable region sequence is shown in uppercase, while constant region sequence is shown in lowercase, and with corresponding CDR regions underlined;

FIG. 2C shows the DNA sequence of the MOR-6990 light chain (SEQ ID NO:14), where variable region sequence is shown in uppercase, while constant region sequence is shown in lowercase, and with corresponding CDR regions underlined;

FIG. 2D shows the amino acid sequence of the MOR-6990 light chain (SEQ ID NO:12), where variable region sequence is shown in uppercase, while constant region sequence is shown in lowercase, and with corresponding CDR regions underlined;

FIG. 2E shows the DNA sequence of the MOR-6991 heavy chain (SEQ ID NO:27), where variable region sequence is shown in uppercase, while constant region sequence is shown in lowercase, and with corresponding CDR regions underlined;

FIG. 2F shows the amino acid sequence of the MOR-6991 heavy chain (SEQ ID NO:25), where variable region sequence is shown in uppercase, while constant region sequence is shown in lowercase, and with corresponding CDR regions underlined;

FIG. 2G shows the DNA sequence of the MOR-6991 light chain (SEQ ID NO:28), where variable region sequence is shown in uppercase, while constant region sequence is shown in lowercase, and with corresponding CDR regions underlined;

FIG. 2H shows the amino acid sequence of the MOR-6991 light chain (SEQ ID NO:26), where variable region sequence is shown in uppercase, while constant region sequence is shown in lowercase, and with corresponding CDR regions underlined;

FIG. 2I shows the DNA sequence of the MOR-6993 heavy chain (SEQ ID NO:41), where variable region sequence is shown in uppercase, while constant region sequence is shown in lowercase, and with corresponding CDR regions underlined;

FIG. 2J shows the amino acid sequence of the MOR-6993 heavy chain (SEQ ID NO:39), where variable region sequence is shown in uppercase, while constant region sequence is shown in lowercase, and with corresponding CDR regions underlined;

FIG. 2K shows the DNA sequence of the MOR-6993 light chain (SEQ ID NO:42), where variable region sequence is shown in uppercase, while constant region sequence is shown in lowercase, and with corresponding CDR regions underlined;

FIG. 2L shows the amino acid sequence of the MOR-6993 light chain (SEQ ID NO:40), where variable region sequence is shown in uppercase, while constant region sequence is shown in lowercase, and with corresponding CDR regions underlined;

FIG. 2M shows the DNA sequence of the MOR-6990-GL heavy chain (SEQ ID NO:60), where germ line mutations are shown by boxing, and corresponding CDR regions underlined; variable region sequence is shown in uppercase, while constant region sequence is lower case;

FIG. 2N shows the amino acid sequence of the MOR-6990-GL heavy chain (SEQ ID NO:58), where germ line mutations are shown by boxing, and corresponding CDR regions underlined; variable region sequence is shown in uppercase, while constant region sequence is lower case;

FIG. 2O shows the DNA sequence of the MOR-6990-GL light chain (SEQ ID NO:61), where germ line mutations are shown by boxing, and corresponding CDR regions underlined; variable region sequence is shown in uppercase, while constant region sequence is lower case;

FIG. 2P shows the amino acid sequence of the MOR-6990-GL light chain (SEQ ID NO:59), where germ line mutations are shown by boxing, and corresponding CDR regions underlined; variable region sequence is shown in uppercase, while constant region sequence is lower case;

FIG. 2Q shows the DNA sequence of the MOR-6991-GL heavy chain (SEQ ID NO:64), where germ line mutations are shown by boxing, and corresponding CDR regions underlined; variable region sequence is shown in uppercase, while constant region sequence is lower case;

FIG. 2R shows the amino acid sequence of the MOR-6991-GL heavy chain (SEQ ID NO:62), where germ line mutations are shown by boxing, and corresponding CDR regions underlined; variable region sequence is shown in uppercase, while constant region sequence is lower case;

FIG. 2S shows the DNA sequence of the MOR-6991-GL light chain (SEQ ID NO:65), where germ line mutations are shown by boxing, and corresponding CDR regions underlined; variable region sequence is shown in uppercase, while constant region sequence is lower case;

FIG. 2T shows the amino acid sequence of the MOR-6991-GL light chain (SEQ ID NO:63), where germ line mutations are shown by boxing, and corresponding CDR regions underlined; variable region sequence is shown in uppercase, while constant region sequence is lower case;

FIG. 2U shows the DNA sequence of the MOR-6993-GL heavy chain (SEQ ID NO:68), where germ line mutations are shown by boxing, and corresponding CDR regions underlined; variable region sequence is shown in uppercase, while constant region sequence is lower case;

FIG. 2V shows the amino acid sequence of the MOR-6993-GL heavy chain (SEQ ID NO:66), where germ line mutations are shown by boxing, and corresponding CDR regions underlined; variable region sequence is shown in uppercase, while constant region sequence is lower case;

FIG. 2W shows the DNA sequence of the MOR-6993-GL light chain (SEQ ID NO:69), where germ line mutations are shown by boxing, and corresponding CDR regions underlined; variable region sequence is shown in uppercase, while constant region sequence is lower case;

FIG. 2X shows the amino acid sequence of the MOR-6993-GL light chain (SEQ ID NO:67), where germ line mutations are shown by boxing, and corresponding CDR regions underlined; variable region sequence is shown in uppercase, while constant region sequence is lower case;

FIG. 3 shows the amino acid sequence of human OPN (SEQ ID NO:43), isoform b (Genbank NP_{—}000573).

FIG. 4A shows the amino acid sequence of the MOR-10475 light chain variable region (SEQ ID NO:76)—CDR regions are underlined;

FIG. 4B shows the DNA sequence of the MOR-10475 light chain variable region—corresponding CDR regions are underlined (SEQ ID NO:77);

FIG. 4C shows the amino acid sequence of the MOR-10475 light chain (SEQ ID NO:78), where variable region sequence is shown in uppercase, while constant region sequence is shown in lowercase, and with corresponding CDR regions underlined;

FIG. 5A shows the effect of MOR-6993 on tumor weight in a preclinical model of breast cancer;

FIG. 5B shows the effect of MOR-6993 on metastasis in a preclinical model of breast cancer;

FIG. 6A shows the neutralization of mouse osteopontin by MOR-6990 and MOR-6993;

FIG. 6B shows the neutralization of human osteopontin by MOR-6990 and MOR-6993.

### DETAILED DESCRIPTION

The present disclosure is based on the discovery of novel antibodies that have a high affinity for osteopontin and can deliver a therapeutic benefit to a subject. The antibodies of the present disclosure, which may be human, or humanized, can be used in many contexts, which are more fully described herein.

In order that the present disclosure may be more readily understood, certain terms are first defined. Additional definitions are set forth throughout the detailed description.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art.

The methods and techniques of the present disclosure are generally performed according to methods well known in the art and as described in various general and more specific references that are cited and discussed throughout the present specification unless otherwise indicated. Such references include, e.g., Sambrook and Russell, Molecular Cloning, A Laboratory Approach, Cold Spring Harbor Press, Cold Spring Harbor, N.Y. (2001), Ausubel et al., Current Protocols in Molecular Biology, John Wiley & Sons, NY (2002), and Harlow and Lane Antibodies: A Laboratory Manual, Cold Spring Harbor Laboratory Press, Cold Spring Harbor, N.Y. (1990). Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)).

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). The CDR regions can be determined using the Kabat or Chothia numbering systems, both of which are well known to those of skill in the art. See, e.g. Kabat, E. A., et al. (1991) Sequences of Proteins of Immunological Interest, Fifth Edition, U.S. Department of Health and Human Services, NIH Publication No. 91-3242; Chothia and Lesk, J. Mol. Biol. 196:901-917 (1987). Each V_{H} and V_{L} is composed of three CDRs and four FRs, arranged from amino-terminus to carboxy-terminus in the following order: FR1, CDR1, FR2, CDR2, FR3, CDR3, FR4. Throughout the present disclosure, the three CDRs of the heavy chain are referred to as H-CDR1, H-CDR2, and H-CDR3. Similarly, the three CDRs of the light chain are referred to as L-CDR1, L-CDR2, and L-CDR3. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. Within light and heavy chains, the variable and constant regions are joined by a “J” region of about 12 or more amino acids, with the heavy chain also including a “D” region of about 10 or more amino acids. See generally, Fundamental Immunology Ch. 7 (Paul, W., ed., 2^{nd} ed. Raven Press, N.Y. (1989)).

A “human” antibody, or antigen-binding portion thereof, is hereby defined as one that is not chimeric (e.g., not “humanized”) and not from (either in whole or in part) a non-human species. A human antibody or antigen-binding portion can be derived from a human or can be a synthetic human antibody. A “synthetic human antibody” is defined herein as an antibody having a sequence derived, in whole or in part, in silico from synthetic sequences that are based on the analysis of known human antibody sequences. In silico design of a human antibody sequence or fragment thereof can be achieved, for example, by analyzing a database of human antibody or antibody fragment sequences and devising a polypeptide sequence utilizing the data obtained therefrom. Another example of a human antibody or antigen-binding portion, is one that is encoded by a nucleic acid isolated from a library of antibody sequences of human origin (i.e., such library being based on antibodies taken from a human natural source).

A “humanized antibody”, or antigen-binding portion thereof, is defined herein as one that is (i) derived from a non-human source (e.g., a transgenic mouse which bears a heterologous immune system), which antibody is based on a human germline sequence; or (ii) chimeric, wherein the variable domain is derived from a non-human origin and the constant domain is derived from a human origin or (iii) CDR-grafted, wherein the CDRs of the variable domain are from a non-human origin, while one or more frameworks of the variable domain are of human origin and the constant domain (if any) is of human origin. In the case where the CDRs are grafted from a non-human origin, said CDRs can be subsequently altered in order to improve the binding affinity to the target of interest.

As used herein, an antibody “specifically binds”, “specifically binds to,” is “specific to/for” or “specifically recognizes” an antigen (here, osteopontin) if such antibody is able to discriminate between such antigen and one or more reference antigen(s), since binding specificity is not an absolute, but a relative property. In its most general form (and when no defined reference is mentioned), “specific binding” is referring to the ability of the antibody to discriminate between the antigen of interest and an unrelated antigen, as determined, for example, in accordance with one of the following methods. Such methods comprise, but are not limited to Western blots, ELISA-, RIA-, ECL-, IRMA-tests and peptide scans. For example, a standard ELISA assay can be carried out. The scoring may be carried out by standard color development (e.g. secondary antibody with horseradish peroxide and tetramethyl benzidine with hydrogenperoxide). The reaction in certain wells is scored by the optical density, for example, at 450 nm. Typical background (=negative reaction) may be 0.1 OD; typical positive reaction may be 1 OD. This means the difference positive/negative can be more than 10-fold. Typically, determination of binding specificity is performed by using not a single reference antigen, but a set of about three to five unrelated antigens, such as milk powder, BSA, transferrin or the like. As used above, corresponding antigens from different species are considered “related”, and are thus not “unrelated”. For example, unless indicated otherwise, an antibody or antigen-binding portion described herein that binds both murine and human OPN is considered to “bind specifically” to OPN, provided that such antibody or antigen-binding portion does not also bind antigens that are “unrelated”, as described above. Typically, a specific or selective reaction will be at least twice the background signal or noise and more typically more than 10 times the background, even more specifically, an antibody is said to “specifically bind” an antigen when the equilibrium dissociation constant (K_{D}) is ≦1 μM, for example ≦100 nM and, further for example, ≦10 nM.

The term “kₒₙ”, as used herein, is intended to refer to the on-rate, or association rate of a particular antibody-antigen interaction, whereas the term “k_{off},” as used herein, is intended to refer to the off-rate, or dissociation rate of a particular antibody-antigen interaction. The term “K_{D}”, as used herein, is intended to refer to the dissociation constant, which is obtained from the ratio of k_{off} to kₒₙ (i.e., k_{off}/kₒₙ) and is expressed as a molar concentration (M). K_{D} values for antibodies can be determined using methods well established in the art. One method for determining the K_{D} of an antibody is by using surface plasmon resonance, typically using a biosensor system such as a Biacore® system.

The term “compete”, as used herein with regard to an antibody, refers to when a first antibody, or an antigen-binding portion thereof, competes for binding with a second antibody, or an antigen-binding portion thereof, where binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). For instance, cross-competing antibodies can bind to the epitope, or portion of the epitope, to which the antibodies as disclosed herein bind. Use of both competing and cross-competing antibodies is encompassed by the present disclosure. Regardless of the mechanism by which such competition or cross-competition occurs (e.g., steric hindrance, conformational change, or binding to a common epitope, or portion thereof, and the like), the skilled artisan would appreciate, based upon the teachings provided herein, that such competing and/or cross-competing antibodies are encompassed and can be useful for the methods disclosed herein.

Also, as used herein, an “immunoglobulin” (Ig) is defined as a protein belonging to the class, or isotype, IgG, IgM, IgE, IgA, or IgD (or any subclass thereof), and includes all conventionally known antibodies and antigen-binding portions thereof.

As used herein, “isotype” or “class” refers to the antibody class (e.g., IgM or IgG) that is encoded by the heavy chain constant region genes. The constant domains of antibodies are not involved in binding to antigen, but exhibit various effector functions. Depending on the amino acid sequence of the heavy chain constant region, a given human antibody or immunoglobulin can be assigned to one of five major classes of immunoglobulins: IgA, IgD, IgE, IgG, and IgM. The structures and three-dimensional configurations of different classes of immunoglobulins are well-known. Of the various human immunoglobulin classes, only human IgG1, IgG2, IgG3, IgG4, and IgM are known to activate complement. Human IgG1 and IgG3 are known to mediate ADCC in humans.

As used herein, “subclass” refers to the further specification within an isotype of the heavy chain constant region gene, such as, for example, the IgG1, IgG2, IgG3, or IgG4 subclasses within the IgG isotype.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. Conformational and nonconformational epitopes are distinguished in that the binding to the former, but not the latter, is lost in the presence of denaturing solvents.

The term “antigen-binding portion” of an antibody (or simply “antibody portion”), as used herein, refers to one or more fragments of an antibody that retain the ability to specifically bind to an antigen (e.g., OPN). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., Nature 341:544-546 (1989)), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv). Such single chain antibodies are also intended to be encompassed within the term “antigen-binding portion” of an antibody. These antibody fragments may be obtained using any suitable technique, including conventional techniques known to those with skill in the art, and the fragments may be screened for utility in the same manner as are intact antibodies.

An “isolated antibody”, as used herein, is intended to refer to an antibody that is substantially free of other antibodies having different antigenic specificities (e.g., an isolated antibody that specifically binds OPN is substantially free of antibodies that specifically bind antigens other than OPN). An isolated antibody that specifically binds OPN may, however, have cross-reactivity to other antigens, such as OPN molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “sequence identity” between two polypeptide sequences indicates the percentage of amino acids that are identical between the sequences. The amino acid sequence identity of polypeptides can be determined conventionally using known computer programs such as Bestfit, FASTA, or BLAST (see, e.g. Pearson, Methods Enzymol. 183:63-98 (1990); Pearson, Methods Mol. Biol. 132:185-219 (2000); Altschul et al., J. Mol. Biol. 215:403-410 (1990); Altschul et al., Nucelic Acids Res. 25:3389-3402 (1997)). When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, fragments, or variants thereof disclosed herein.

“Glycoform” refers to a complex oligosaccharide structure comprising linkages of various carbohydrate units. Such structures are described in, e.g., Essentials of Glycobiology Varki et al., eds., Cold Spring Harbor Laboratory Press, Cold Spring Harbor, N.Y. (1999), which also provides a review of standard glycobiology nomenclature. Such glycoforms include, but are not limited to, G2, G1, G0, G-1, and G-2 (see, e.g., International Patent Publication No. WO 99/22764).

“Glycosylation pattern” is defined as the pattern of carbohydrate units that are covalently attached to a protein (e.g., the glycoform) as well as to the site(s) to which the glycoform(s) are covalently attached to the peptide backbone of a protein, more specifically to an immunoglobulin protein. It is likely that antibodies expressed by different cell lines or in transgenic animals will have different glycoforms and/or glycosylation patterns compared with each other. However, all antibodies encoded by the nucleic acid molecules provided herein, or comprising the amino acid sequences provided herein are part of the present disclosure, regardless of the glycosylation of such antibodies.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc.

As used herein, the terms “treat”, “treating”, or “treatment”, with reference to a certain disease condition, mean reducing the frequency with which symptoms of the disease (i.e., tumor growth and/or metastasis, or other effect mediated by the numbers and/or activity of immune cells, and the like) are experienced by a subject. These terms include the administration of the compounds or agents of the present disclosure to prevent or delay the onset of the symptoms, complications, or biochemical indicia of the disease, or to alleviate the symptoms or arrest or inhibit further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

As used herein, the term “compound” or “pharmaceutical compound” includes antibodies, antigen-binding portions thereof, immunoconjugates, and bispecific molecules.

### Antibodies of the Disclosure

Antibodies of the disclosure may be derived from a recombinant antibody library that is based on amino acid sequences that have been designed in silico and encoded by nucleic acids that are synthetically created. In silico design of an antibody sequence is achieved, for example, by analyzing a database of human sequences and devising a polypeptide sequence utilizing the data obtained therefrom. Methods for designing and obtaining in silico-created sequences are described, for example, in Knappik et al., J. Mol. Biol. (2000) 296:57; Krebs et al., J. Immunol. Methods. (2001) 254:67; and U.S. Pat. No. 6,300,064 issued to Knappik et al.

Throughout this disclosure, reference is made to the following representative antibodies of the disclosure: MOR-6990 (6990), MOR-6991 (6991), MOR-6993 (6993), and MOR-10475 (10475). As further described in Example 5, 6990 represents an antibody having a variable heavy region corresponding to SEQ ID NO:7, and a variable light region corresponding to SEQ ID NO:8; 6991 represents an antibody having a variable heavy region corresponding to SEQ ID NO:21, and a variable light region corresponding to SEQ ID NO:22; and 6993 represents an antibody having a variable heavy region corresponding to SEQ ID NO:35, and a variable light region corresponding to SEQ ID NO:36. 10475 represents an antibody having a variable heavy region corresponding to SEQ ID NO:7, and a variable light region corresponding to SEQ ID NO:76.

The amino acid CDR sequences for the 6990, 6991, 6993, and 10475 representative antibodies are shown below in Table 1.

TABLE 1
CDR sequences of antibodies 6990, 6991, and 6993.

| | | |
|---|---|---|
| MOR-6990 | H-CDR1: SNYVMH | (SEQ ID NO: 1) |
| | H-CDR2: SIFGSGSDTYYADSVKG | (SEQ ID NO: 2) |
| | H-CDR3: RSASSGFGFAGYGIDS | (SEQ ID NO: 3) |
| | L-CDR1: SGDSLRYYYAH | (SEQ ID NO: 4) |
| | L-CDR2: DDNKRPS | (SEQ ID NO: 5) |
| | L-CDR3: QSWDLFHSSV | (SEQ ID NO: 6) |
| MOR-6991 | H-CDR1: NNYAVS | (SEQ ID NO: 15) |
| | H-CDR2: GISYGGSNTYYADSVKG | (SEQ ID NO: 16) |
| | H-CDR3: RTLGGDFDH | (SEQ ID NO: 17) |
| | L-CDR1: SGSSSNIGSNYVN | (SEQ ID NO: 18) |
| | L-CDR2: GNSKRPS | (SEQ ID NO: 19) |
| | L-CDR3: QSFTQMLLV | (SEQ ID NO: 20) |
| MOR-6993 | H-CDR1: TTSSMH | (SEQ ID NO: 29) |
| | H-CDR2: RISSHGSNTYYADSVKG | (SEQ ID NO: 30) |
| | H-CDR3: RDMYRGVYGFAL | (SEQ ID NO: 31) |
| | L-CDR1: SGDAIRNYYVH | (SEQ ID NO: 32) |
| | L-CDR2: EDSDRPS | (SEQ ID NO: 33) |
| | L-CDR3: QSYDKSNVV | (SEQ ID NO: 34) |
| MOR-10475 | H-CDR1: SNYVMH | (SEQ ID NO: 1) |
| | H-CDR2: SIFGSGSDTYYADSVKG | (SEQ ID NO: 2) |
| | H-CDR3: RSASSGFGFAGYGIDS | (SEQ ID NO: 3) |
| | L-CDR1: SGDSLRYYYAH | (SEQ ID NO: 4) |
| | L-CDR2: DDNKRPS | (SEQ ID NO: 5) |
| | L-CDR3: QAWDLINSHV | (SEQ ID NO: 75) |

In one aspect, the disclosure provides antibodies having an antigen-binding region that can bind specifically to or has a high affinity for, one or more regions of human osteopontin, whose amino acid sequence is set forth in SEQ ID NO:43, and in FIG. 3. An antibody is said to have a “high affinity” for an antigen if the affinity measurement is at least 100 nM (monovalent affinity of Fab fragment). An antibody or antigen-binding portion of the present disclosure typically binds to human osteopontin with an affinity of about less than 500 nM, for example less than about 100 nM, less than about 60 nM, less than about 30 nM, less than about 10 nM, or less than about 3 nM. Exemplary antibodies of the present disclosure and their corresponding binding affinities for osteopontin are further described in Examples 2 and 3 herein.

The present disclosure also provides CDR portions of antibodies to osteopontin (including Chothia and Kabat CDRs). Determination of CDR regions is well within the skill of the art. It is understood that in some embodiments, CDRs can be a combination of the Kabat and Chothia CDR (also termed “combined CDRs” or “extended CDRs”). In some embodiments, the CDRs are the Kabat CDRs. In other embodiments, the CDRs are the Chothia CDRs. In other words, in embodiments with more than one CDR, the CDRs may be any of Kabat, Chothia, combination CDRs, or combinations thereof.

An antibody of the present disclosure can be species cross-reactive with humans and at least one other species, which may be murine or rat. An antibody that is cross reactive with at least one osteopontin species, for example, can provide greater flexibility and benefits over known anti-osteopontin antibodies, for purposes of conducting in vivo studies in multiple species with the same antibody.

Preferably, an antibody of the disclosure not only is able to bind to OPN, but also is able to reduce tumor cell metastasis and/or reduce abnormal cell growth, such as cancer.

### Antibodies Having Particular Germline Sequences

In certain aspects, an antibody of the disclosure comprises a heavy chain variable region from a particular germline heavy chain immunoglobulin gene and/or a light chain variable region from a particular germline light chain immunoglobulin gene.

For example, in one aspect, the disclosure provides an isolated monoclonal antibody, or an antigen-binding portion thereof, comprising a heavy chain variable region that is the product of, or derived from, a human V_{H} 3-23 gene, wherein the antibody specifically binds OPN. In yet another aspect, the disclosure provides an isolated monoclonal antibody, or an antigen-binding portion thereof, comprising a light chain variable region that is the product of, or derived from, a human V_{L} λ3, or λ1-13 gene, wherein the antibody specifically binds OPN. In yet another illustrative aspect, the disclosure provides an isolated monoclonal antibody, or antigen-binding portion thereof, wherein the antibody:

(a) comprises a heavy chain variable region that is the product of, or derived from, a human V_{H} 3-23 gene (which gene encodes the amino acid sequences set forth in SEQ ID NOs: 7, 21, and 35);

(b) comprises a light chain variable region that is the product of, or derived from, a human V_{L} λ3, or λ1-13 gene (which genes encodes the amino acid sequences set forth in SEQ ID NOs: 8, 36, or 22, respectively); and

(c) specifically binds to OPN, preferably human OPN.

Examples of antibodies having V_{H} and V_{L} of V_{H} 3-23 and V_{L} λ3, respectively, are 6990 and 6993. An example of an antibody having V_{H} and V_{L} of V_{H} 3-23 and V_{L} λ1-13, respectively, is 6991.

As used herein, a human antibody comprises heavy or light chain variable regions that is “the product of” or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a selected human antibody typically is at least 90% identical in amino acid sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. In certain cases, the human antibody is identical in amino acid sequence to the amino acid sequence encoded by the germline Ig gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid differences from the amino acid sequence encoded by the germline immunoglobulin gene.

Antibodies that Bind the Same Epitope as the Antibodies of the Disclosure

In another aspect, the disclosure provides antibodies that bind to the same epitope on human OPN as any of the illustrative OPN monoclonal antibodies of the disclosure (i.e., antibodies that have the ability to cross-compete for binding to OPN with any of the monoclonal antibodies of the disclosure). For example, the reference antibody for cross-competition studies can be the monoclonal antibody 6990 (having V_{H} and V_{L} sequences as shown in SEQ ID NOs: 7 and 8, respectively), or the monoclonal antibody 6991 (having V_{H} and V_{L} sequences as shown in SEQ ID NOs: 21 and 22, respectively), or the monoclonal antibody 6993 (having V_{H} and V_{L} sequences as shown in SEQ ID NOs: 35 and 36, respectively). Such cross-competing antibodies can be identified based on their ability to cross-compete with 6990, 6991, or 6993 in standard OPN binding assays. For example, BIAcore analysis, ELISA assays or flow cytometry may be used to demonstrate cross-competition with the illustrative antibodies of the current disclosure. The ability of a test antibody to inhibit the binding of, for example, 6990, 6991, or 6993 to human OPN demonstrates that the test antibody can compete with 6990, 6991, or 6993 for binding to human OPN and thus binds to the same epitope on human OPN as 6990, 6991, or 6993. In one case, the antibody that binds to the same epitope on human OPN as 6990, 6991, or 6993 is a human monoclonal antibody. Such human monoclonal antibodies can be prepared and isolated as described, for example, in the Examples.

### Antibody Variants

Antibodies of the present disclosure are not limited to the specific peptide sequences provided herein. Rather, the disclosure also provides variants of these polypeptides. With reference to the instant disclosure and conventionally available technologies and references, the skilled worker will be able to prepare, test and utilize functional variants of the antibodies disclosed herein, while appreciating that variants having the ability to specifically bind to OPN fall within the scope of the present disclosure.

As used herein, the term “peptide variant” or “antibody variant” encompasses both conservative and non-conservative substitutions, additions, and deletions, and can include, for example, an antibody that has at least one altered CDR (hypervariable) and/or framework (FR) (variable) domain/position, vis-à-vis a peptide sequence disclosed herein. For example, it is well known in the art that the antigen-binding site of an antibody is formed by one or more CDRs, yet the FR regions provide the structural framework for the CDRs and, hence, play an important role in antigen binding. By altering one or more amino acid residues in a CDR or FR region, the skilled worker routinely can generate mutated or diversified antibody sequences, which can be screened against the antigen, for new or improved properties, for example.

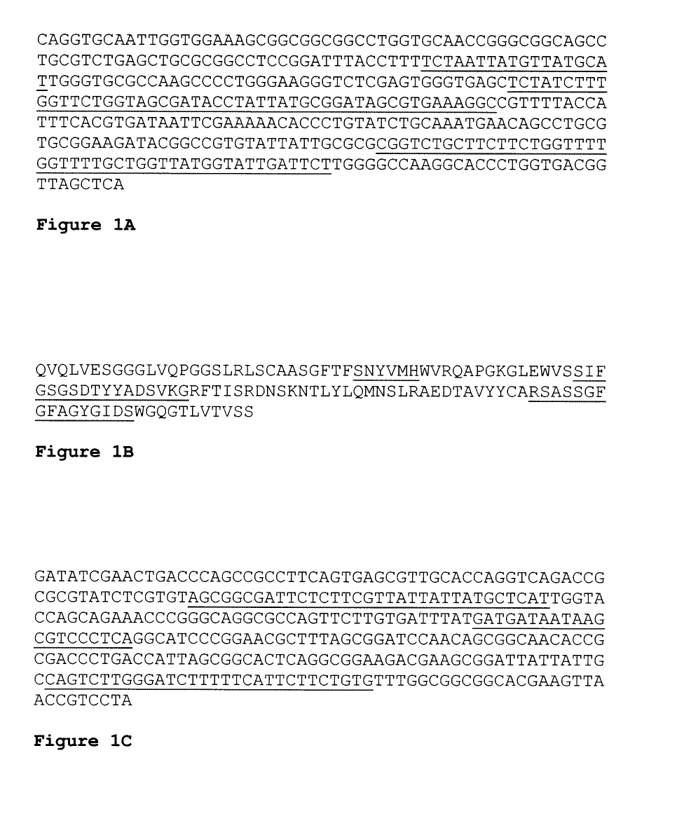

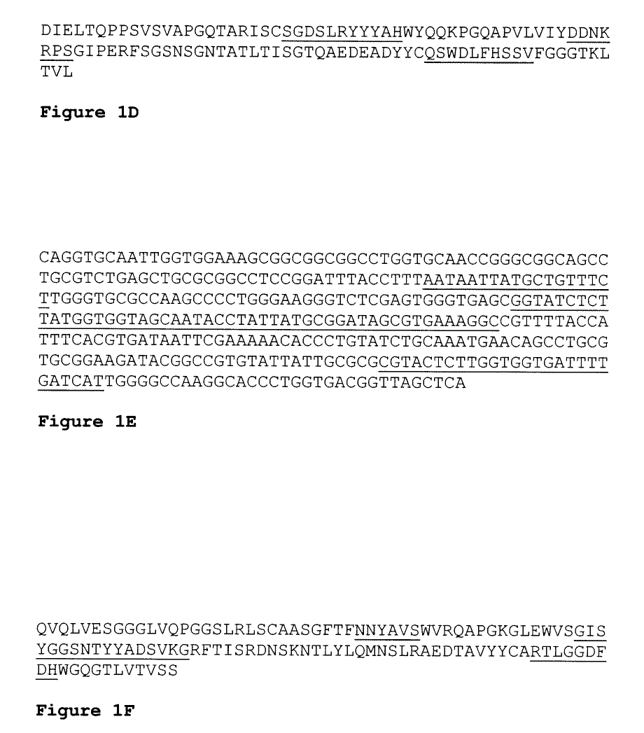

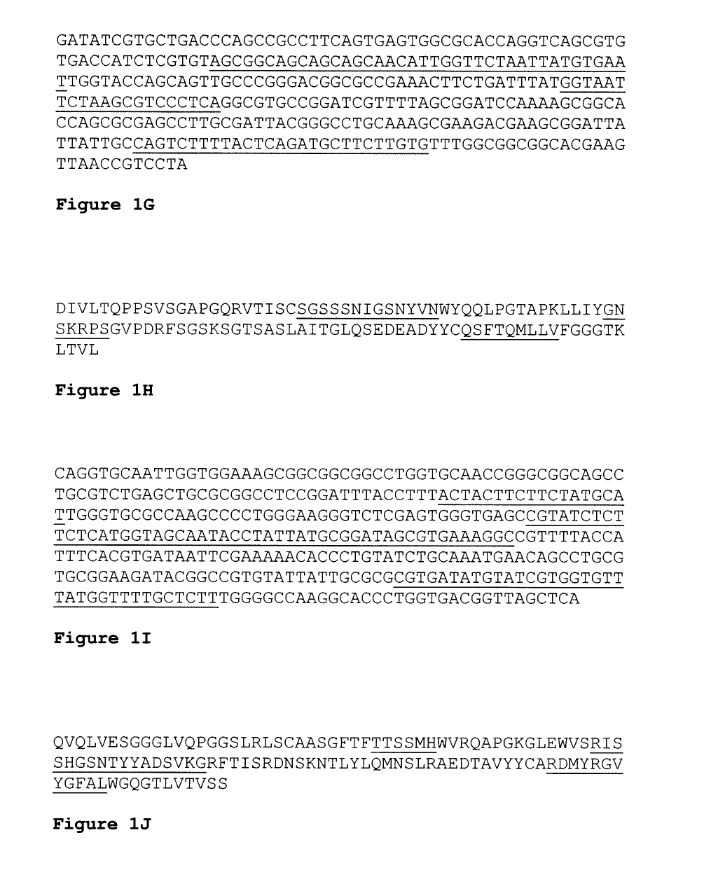

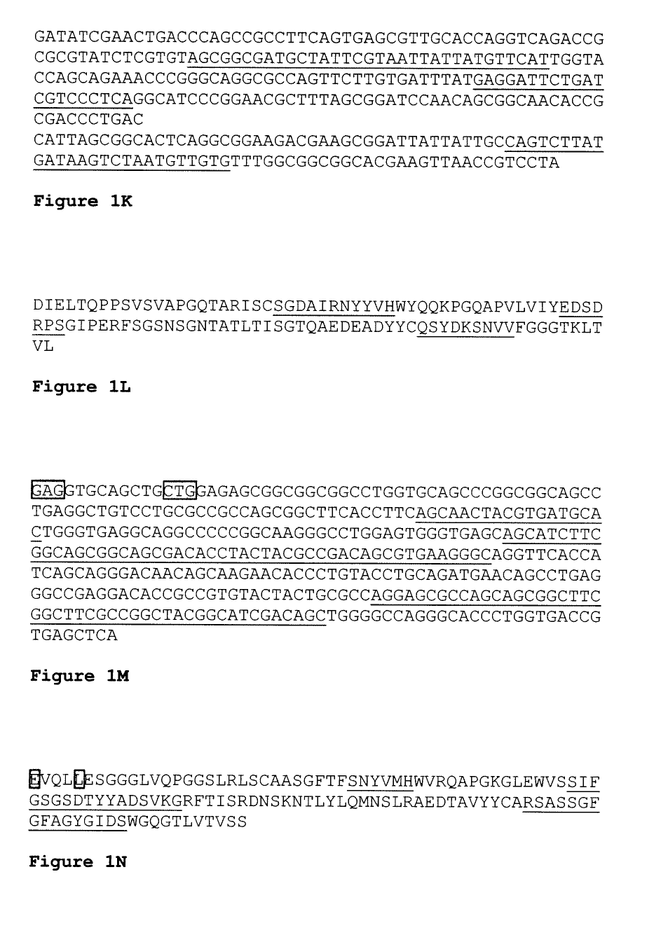

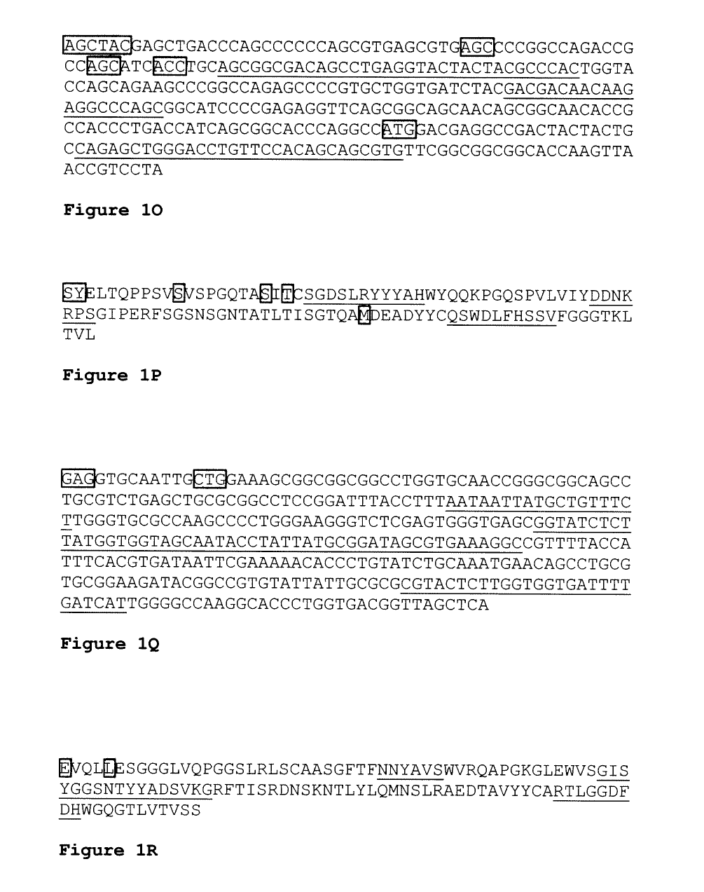

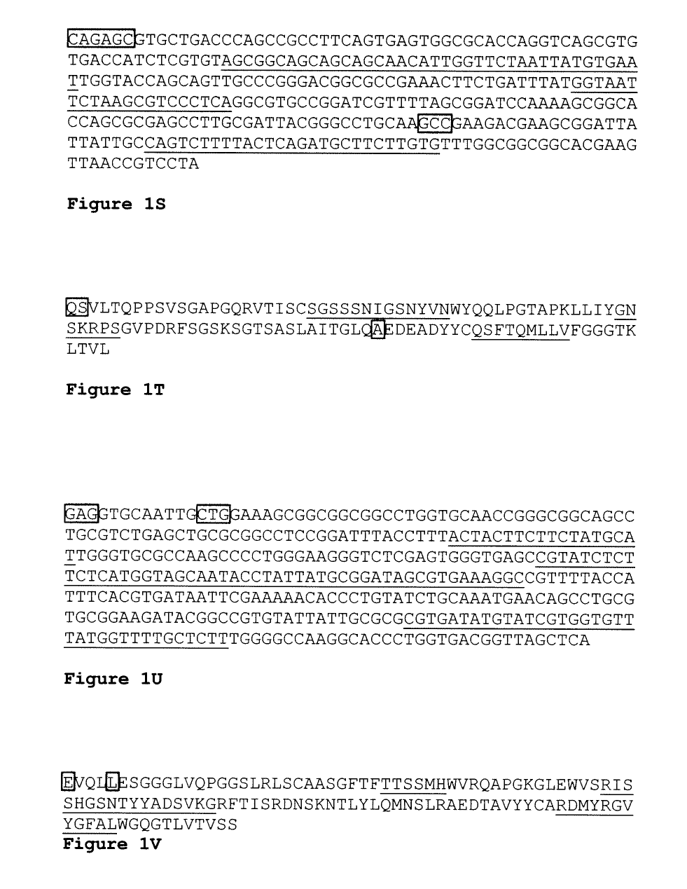

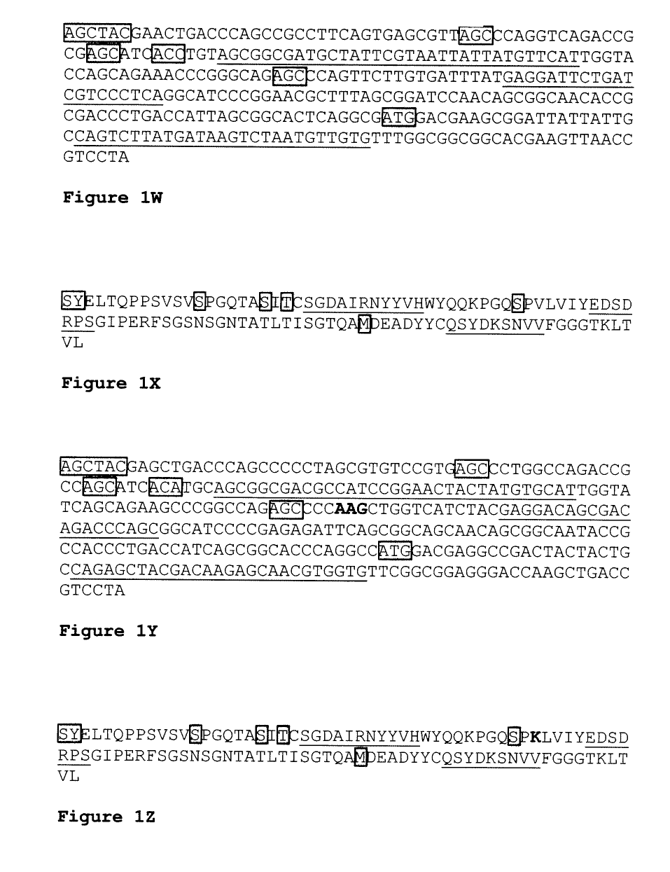

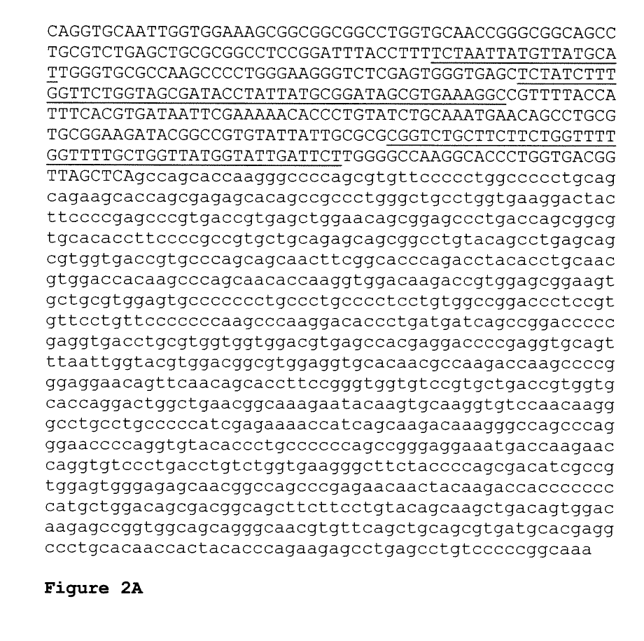

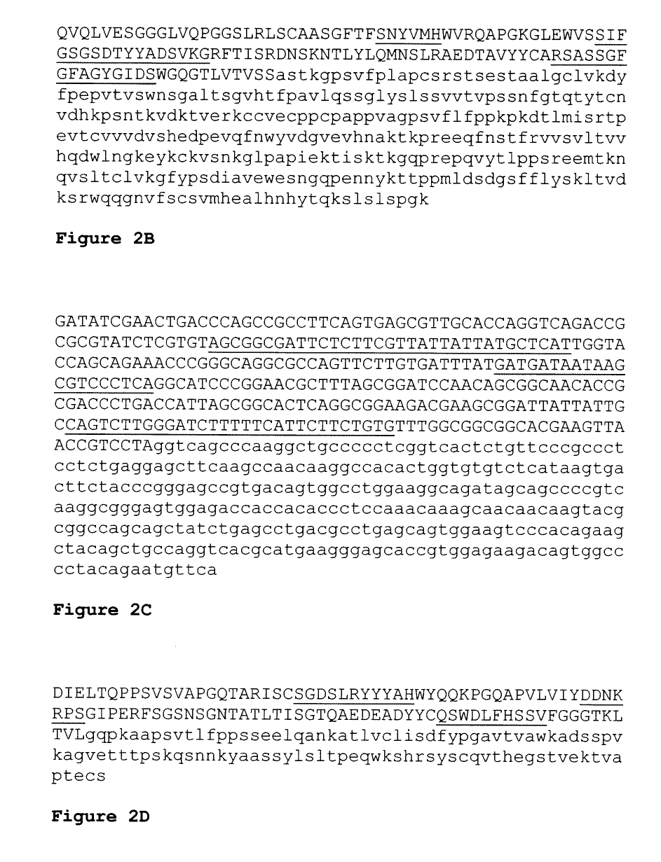

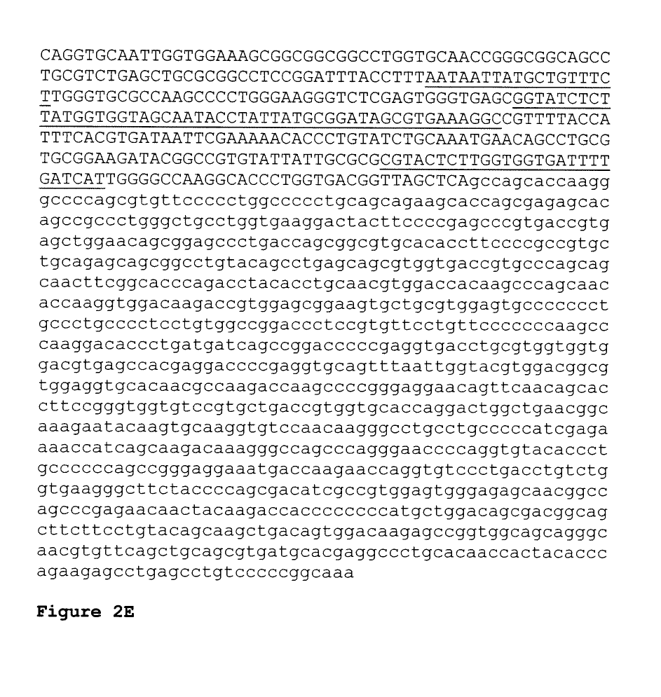

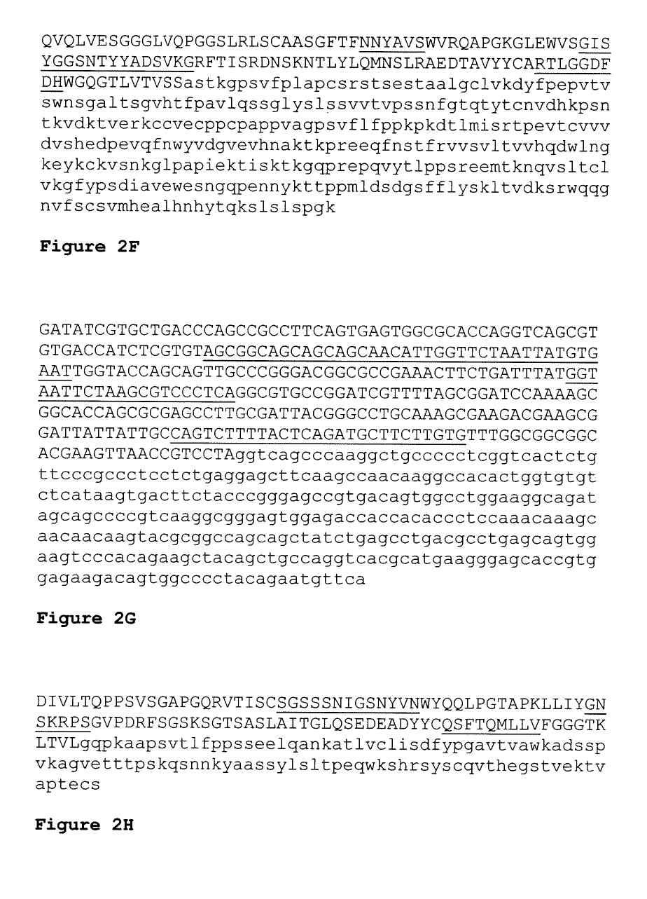

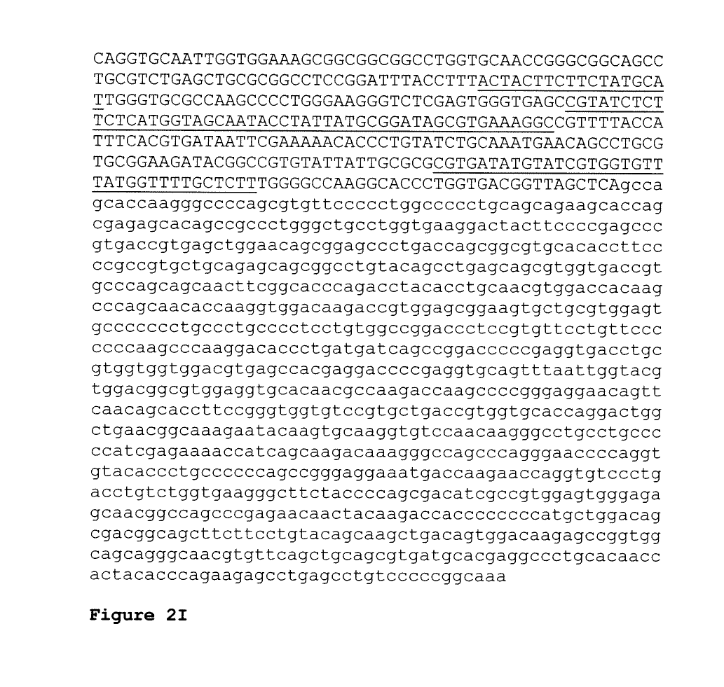

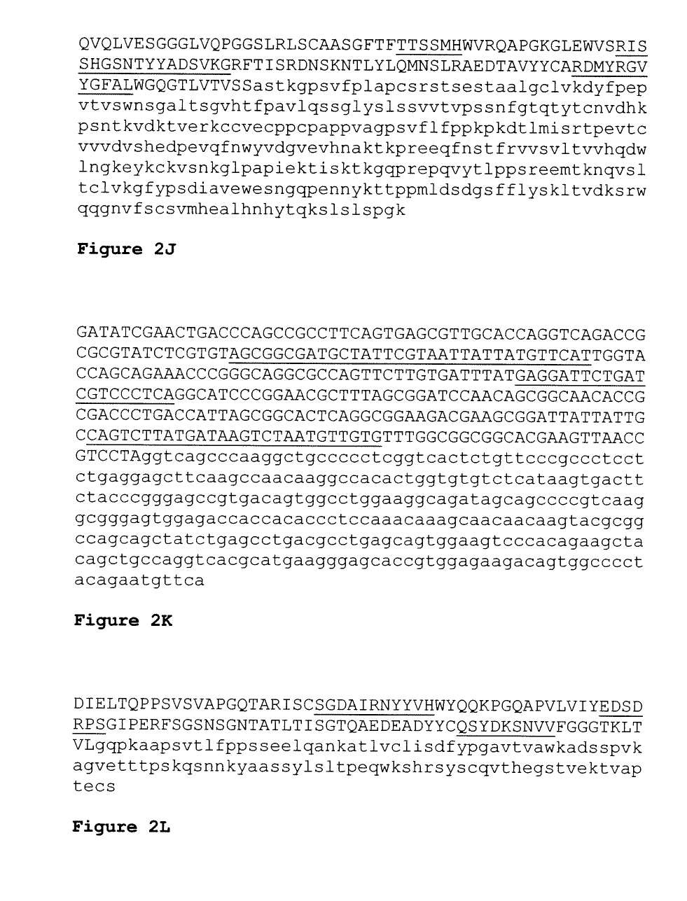

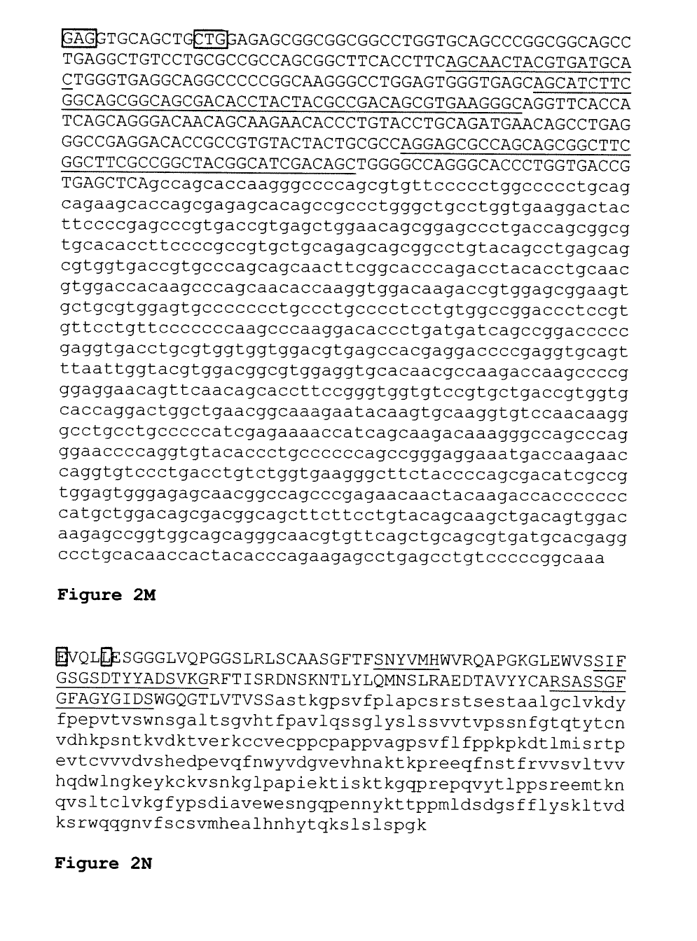

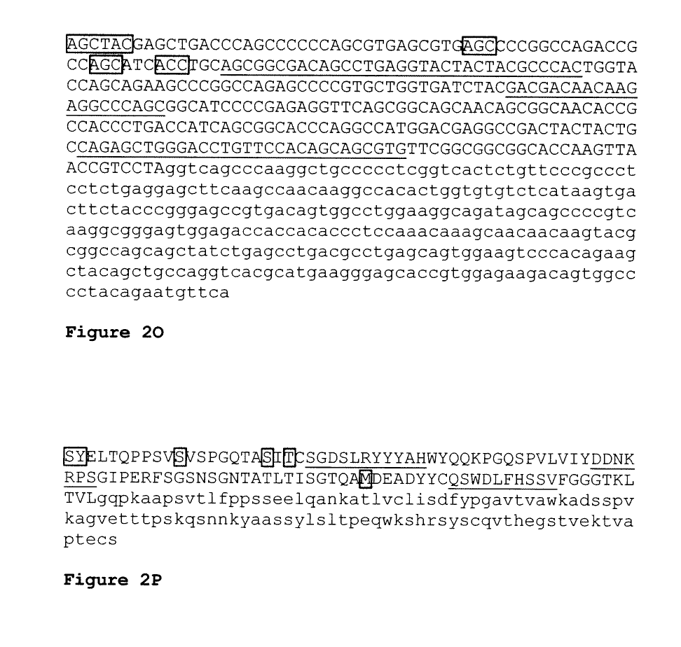

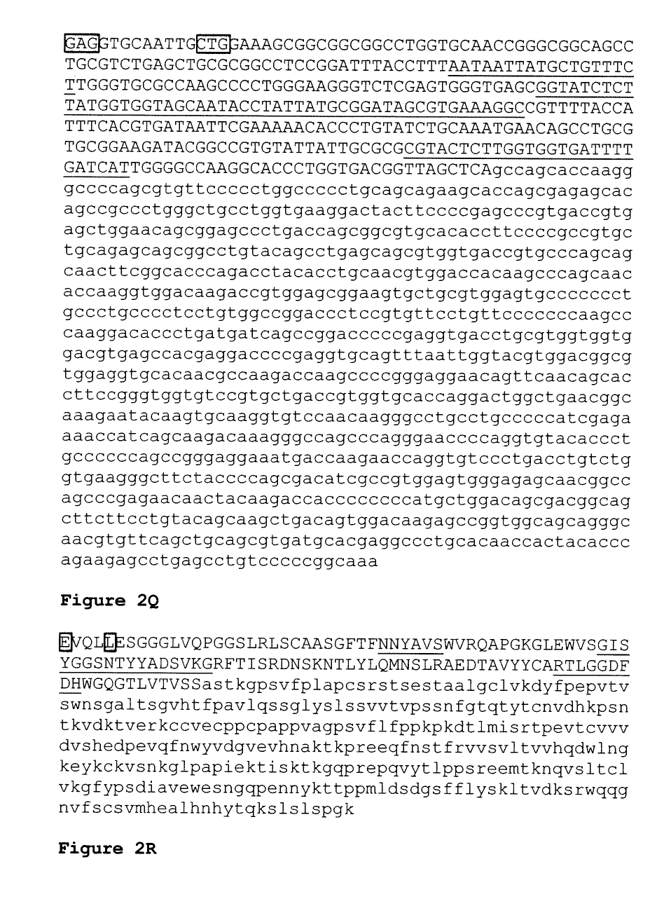

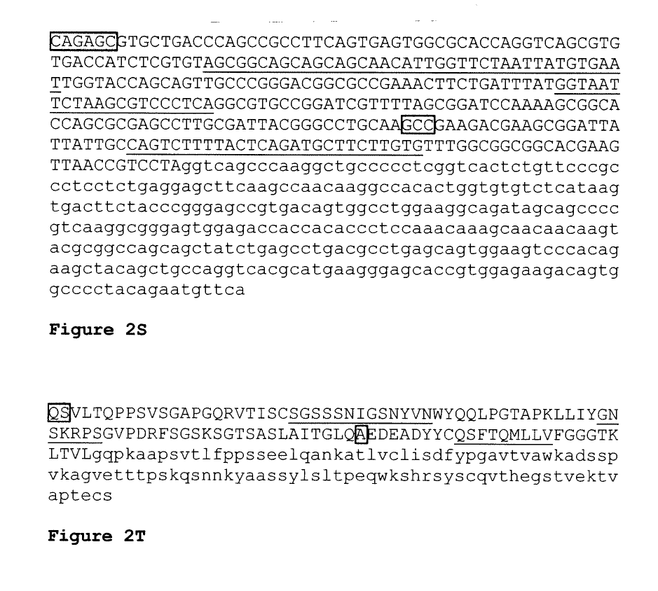

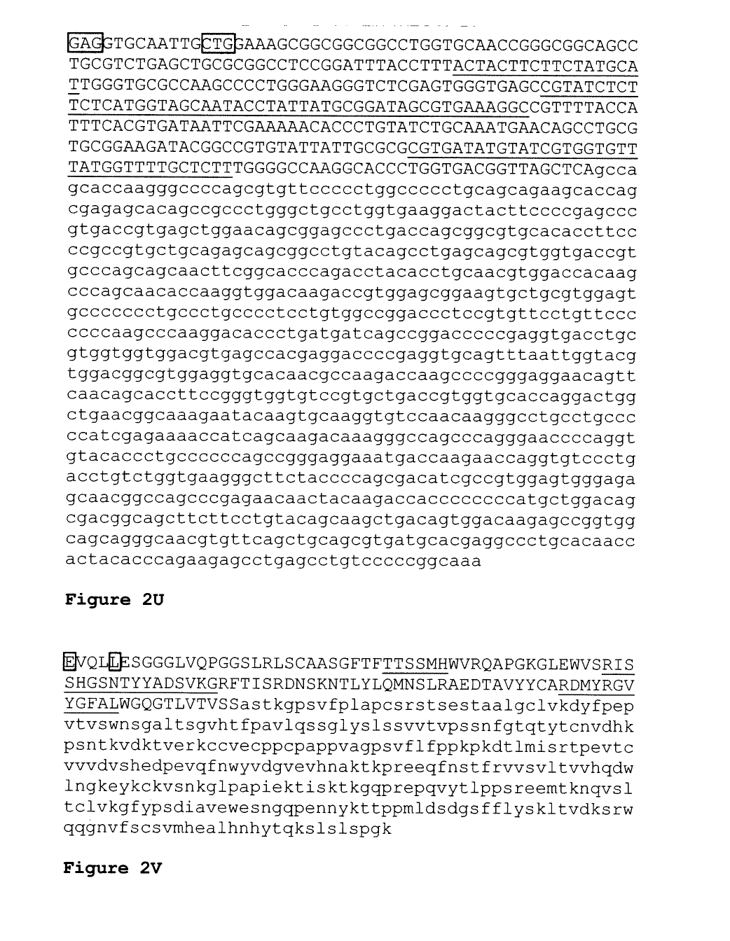

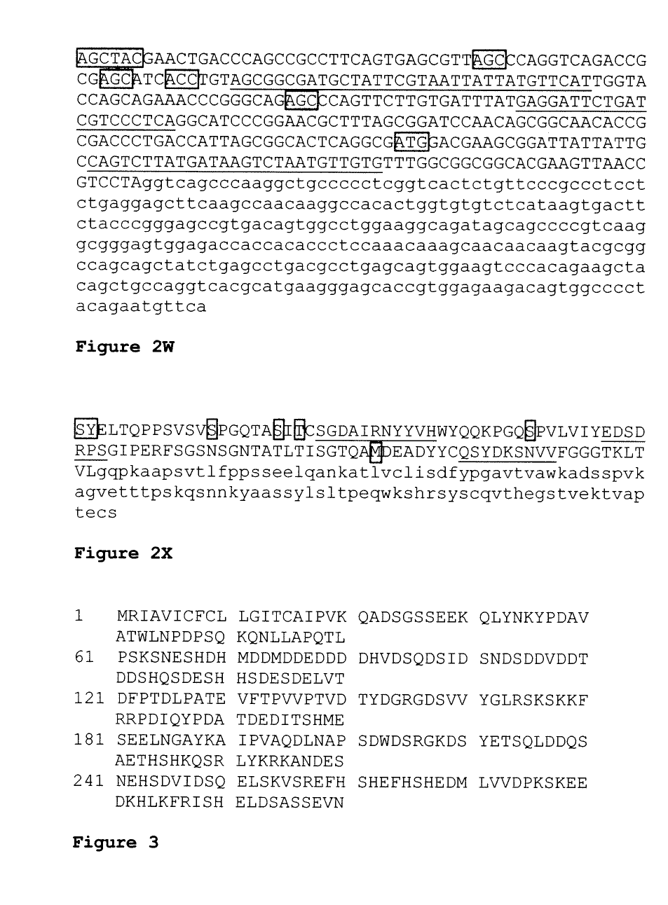

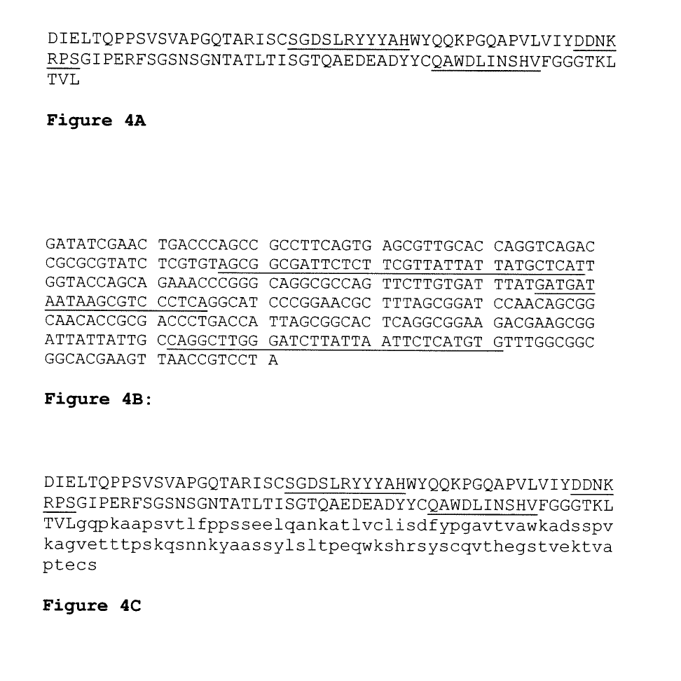

FIGS. 1 and 2 show the VH and VL sequences for certain antibodies of the present disclosure, where the CDR regions are indicated by underline. The skilled worker can use the sequence information described herein to design peptide variants that are within the scope of the present disclosure. For example, variants can be constructed by changing amino acids within one or more CDR regions; a variant might also have one or more altered framework regions. For example, a peptide FR domain might be altered where there is a deviation in a residue compared to a germline sequence.

To determine which amino acid residues to modify, the skilled worker can compare the amino acid sequences disclosed herein to known sequences of the same class of such antibodies, using, for example, the procedure described by Knappik et al., J. Mol. Biol. 296:57 (2000) and U.S. Pat. No. 6,300,064.

For example, variants may be obtained by diversifying one or more amino acid residues in one or more CDRs, and by screening the resulting collection of antibody variants for variants with improved properties. Particularly preferred is diversification of one or more amino acid residues in L-CDR3, H-CDR3, L-CDR1, and/or H-CDR2. Diversification can be done by synthesizing a collection of DNA molecules using trinucleotide mutagenesis (TRIM) technology (Virnekas, et al., Nucl. Acids Res. 22:5600 (1994)). For example, MOR-10475 was obtained by diversifying amino acids in L-CDR3 of MOR-6990.

### Conservative Amino Acid Substitutions

Polypeptide variants may be made that conserve the overall molecular structure of an antibody peptide sequence described herein. Given the properties of the individual amino acids, some rational substitutions will be recognized by the skilled worker. Conservative amino acid substitutions may be made, for instance, on the basis of similarity in polarity, charge, solubility, hydrophobicity, hydrophilicity, and/or the amphipathic nature of the residues involved.

For example, (a) nonpolar (hydrophobic) amino acids include alanine, leucine, isoleucine, valine, proline, phenylalanine, tryptophan, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt α-helices. Similarly, certain amino acids, such as alanine, cysteine, leucine, methionine, glutamic acid, glutamine, histidine and lysine are more commonly found in α-helices, while valine, isoleucine, phenylalanine, tyrosine, tryptophan and threonine are more commonly found in β-pleated sheets. Glycine, serine, aspartic acid, asparagine, and proline are commonly found in turns. Some preferred substitutions may be made among the following groups: (i) S and T; (ii) P and G; and (iii) A, V, L and I. Given the known genetic code, and recombinant and synthetic DNA techniques, the skilled scientist readily can construct DNAs encoding the conservative amino acid variants.

### Engineered and Modified Antibodies

An antibody, or antigen binding portion thereof, of the present disclosure can be prepared using an antibody having one or more of the V_{H} and/or V_{L} sequences disclosed herein as starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. Nature 332:323-327 (1998); Jones, P. et al. Nature 321:522-525 (1986); Queen, C. et al. Proc. Natl. Acad. Sci. U.S.A. 86:10029-10033 (1989); U.S. Pat. No. 5,225,539; and U.S. Pat. Nos. 5,530,101; 5,585,089; 5,693,762 and 6,180,370)

Accordingly, another aspect of the disclosure pertains to isolated antibodies, or antigen binding portions thereof, that contain the V_{H} and V_{L} CDR sequences of the monoclonal antibodies 6990, 6991, and 6993, yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database (Kabat, E. A., et al., Sequences of Proteins of Immunological Interest, Fifth Edition, U.S. Department of Health and Human Services, NIH Publication No. 91-3242 (1991); Tomlinson, I. M., et al., J. Mol. Biol. 227:776-798 (1992); and Cox, J. P. L. et al., Eur. J. Immunol. 24:827-836 (1994)). As another example, the germline DNA sequences for human heavy and light chain variable region genes can be found in the Genbank database.

Framework sequences for use in the antibodies of the disclosure include, but are not limited to, those that are structurally similar to the framework sequences used by selected antibodies of the disclosure, e.g., similar to the V_{H} 3-23 framework sequences and/or the V_{L} λ3 or λ1-13 framework sequences used by illustrative antibodies of the disclosure. For example, the H-CDR1, H-CDR2, and H-CDR3 sequences, and the L-CDR1, L-CDR2, and L-CDR3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

Another type of variant is to mutate amino acid residues within the V_{H} and/or V_{L} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Typically, conservative substitutions (as discussed above) are introduced. The mutations may be amino acid additions and/or deletions. Moreover, typically no more than one, two, three, four or five residues within a CDR region are altered.

Engineered antibodies of the disclosure include those in which modifications have been made to framework residues within the V_{H} and/or V_{L} regions, e.g., to improve the properties of the antibody. Typically such framework variants are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Several of the OPN antibodies of the present disclosure underwent such “back-mutations” to certain germline sequences, as described further in Example 6.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 2003-0153043.

To create an engineered antibody, it is not necessary to actually prepare (i.e., express as a protein) an antibody having one or more of the V_{H} and/or V_{L} sequences provided herein, or one or more CDR regions thereof. Rather, the information contained in the sequence(s) may be used as the starting material to create a “second generation” sequence(s) derived from the original sequence(s) and then the “second generation” sequence(s) is prepared and expressed as a protein. Standard molecular biology techniques can be used to prepare and express the altered antibodies. Preferably, the altered antibody sequence(s) is one that retains one, some or all of the functional properties of the OPN antibodies described herein. The functional properties of the altered antibodies can be assessed using standard assays available in the art and/or described herein, such as those set forth in the Examples.

In certain aspects of the methods of engineering antibodies of the disclosure, mutations can be introduced randomly or selectively along all or part of an OPN antibody coding sequence and the resulting modified OPN antibodies can be screened for binding activity and/or other functional properties as described herein. Mutational methods have been described in the art. For example, PCT Publication WO 02/092780 describes methods for creating and screening antibody mutations using saturation mutagenesis, synthetic ligation assembly, or a combination thereof. Alternatively, PCT Publication WO 03/074679 describes methods of using computational screening methods to optimize physiochemical properties of antibodies.

In addition or alternative to modifications made within the framework or CDR regions, antibodies of the disclosure may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the disclosure may be chemically modified (e.g., one or more chemical moieties can be attached to the antibody) or be modified to alter its glycosylation pattern, again to alter one or more functional properties of the antibody. Each of these aspects is described in further detail below. The numbering of residues in the Fc region is that of the EU index of Kabat.

In one case, the hinge region of CH1 is modified such that the number of cysteine residues in the hinge region is altered, e.g., increased or decreased. This approach is described further in U.S. Pat. No. 5,677,425. The number of cysteine residues in the hinge region of CH1 is altered to, for example, facilitate assembly of the light and heavy chains or to increase or decrease the stability of the antibody.

In another case, the Fc hinge region of an antibody is mutated to decrease the biological half life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745.

In another case, the antibody is modified to increase its biological half life. Various approaches are possible. For example, one or more of the following mutations can be introduced: T252L, T254S, T256F, as described in U.S. Pat. No. 6,277,375. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022.

In yet other cases, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260.

In another case, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551.

In another example, one or more amino acid residues within amino acid positions 231 and 239 are altered to thereby alter the ability of the antibody to fix complement. This approach is described further in PCT Publication WO 94/29351.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072. Moreover, the binding sites on human IgG1 for FcγR1, FcγRII, FcγRIII and FcRn have been mapped and variants with improved binding have been described (see Shields et al., J. Biol. Chem. 276:6591-6604 (2001)). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In still another example, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the disclosure to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8^{-/-} cell lines were created by the targeted disruption of the FUT8 gene in CHO/DG44 cells using two replacement vectors (see U.S. Patent Publication No. 2004-0110704, and Yamane-Ohnuki et al., Biotechnol Bioeng 87:614-22 (2004)). As another example, European Patent Publication No. EP1,176,195 describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. EP1,176,195 also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 describes a variant CHO cell line, Lec13 cells, with reduced ability to attach fucose to Asn(297)-linked carbohydrates, also resulting in hypofucosylation of antibodies expressed in that host cell (see also Shields et al., J. Biol. Chem. 277:26733-26740 (2002)). PCT Publication WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180 (1999)). Alternatively, the fucose residues of the antibody may be cleaved off using a fucosidase enzyme. For example, the fucosidase alpha-L-fucosidase removes fucosyl residues from antibodies (Tarentino et al., (1975) Biochem. 14:5516-23 (1975)).

Another modification of the antibodies herein that is contemplated by the disclosure is pegylation. An antibody can be pegylated to, for example, increase the biological (e.g., serum) half life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Typically, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁ to C₁₀) alkoxy- or aryloxy-polyethylene glycol or polyethylene glycol-maleimide. In certain cases, the antibody to be pegylated is an aglycosylated antibody. Methods for pegylating proteins are known in the art and can be applied to the antibodies of the present disclosure. See for example, European Patent Nos. EP 0154316B1 and EP 0401384B1.

### Production of Monoclonal Antibodies of the Disclosure

Monoclonal antibodies (mAbs) of the present disclosure can be produced by a variety of techniques, including conventional monoclonal antibody methodology e.g., the standard somatic cell hybridization technique of Kohler and Milstein, Nature 256:495 (1975). Other techniques for producing monoclonal antibodies also can be employed, e.g., viral or oncogenic transformation of B lymphocytes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Humanized antibodies of the present disclosure can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using suitable molecular biology techniques. For example, to create a chimeric antibody, the murine variable regions can be linked to human constant regions using methods known in the art (see e.g., U.S. Pat. No. 4,816,567). To create a humanized antibody, the murine CDR regions can be inserted into a human framework using methods known in the art (see e.g., U.S. Pat. No. 5,225,539, and U.S. Pat. Nos. 5,530,101; 5,585,089; 5,693,762 and 6,180,370).

In some cases, the antibodies of the disclosure are human monoclonal antibodies. Such human monoclonal antibodies directed against OPN can be generated using transgenic or transchromosomic mice carrying parts of the human immune system rather than the mouse system. See, e.g. Taylor et al. (1992) Nucleic Acids Research 20:6287-6295 (1992); Chen et al. International Immunology 5: 647-656 (1993); Tuaillon et al., Proc. Natl. Acad. Sci. USA 90:3720-3724 (1993); Choi et al. Nature Genetics 4:117-123 (1993); Chen, et al., EMBO J. 12:821-830 (1993); Tuaillon et al., J. Immunol. 152:2912-2920 (1994); Taylor et al., International Immunology 6:579-591 (1994); and Fishwild et al., Nature Biotechnology 14:845-851 (1996). See further, U.S. Pat. Nos. 5,545,806; 5,569,825; 5,625,126; 5,633,425; 5,789,650; 5,877,397; 5,661,016;

5,814,318; 5,874,299; and 5,770,429; U.S. Pat. No. 5,545,807; and PCT Publication Nos. WO 92/03918, WO 93/12227, WO 94/25585, WO 97/13852, WO 98/24884, WO 01/14424 and WO 99/45962.

In another case, human antibodies of the disclosure can be raised using a mouse that carries human immunoglobulin sequences on transgenes and transchomosomes, such as a mouse that carries a human heavy chain transgene and a human light chain transchromosome. Such mice are described in detail in PCT Publication WO 02/43478.

Still further, alternative transgenic animal systems expressing human immunoglobulin genes are available in the art and can be used to raise OPN antibodies of the disclosure. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963.

Moreover, alternative transchromosomic animal systems expressing human immunoglobulin genes are available in the art and can be used to raise OPN antibodies of the disclosure. For example, mice carrying both a human heavy chain transchromosome and a human light chain transchromosome, referred to as “TC mice” can be used; such mice are described in Tomizuka et al., Proc. Natl. Acad. Sci. USA 97:722-727 (2000). Furthermore, cows carrying human heavy and light chain transchromosomes have been described in the art (Kuroiwa et al., Nature Biotechnology 20:889-894 (2002)) and can be used to raise OPN antibodies of the disclosure.

Human monoclonal antibodies of the disclosure can also be prepared using phage display methods for screening libraries of human immunoglobulin genes. Such phage display methods (e.g. HuCAL® Libraries as described further in Example 1 and herein) for isolating human antibodies are established in the art. See for example: U.S. Pat. Nos. 5,223,409; 5,403,484; 5,571,698; 5,427,908; 5,580,717; 5,969,108; 6,172,197; 5,885,793; 6,521,404; 6,544,731; 6,555,313; 6,582,915 and 6,593,081.

Human monoclonal antibodies of the disclosure can also be prepared using SCID mice into which human immune cells have been reconstituted such that a human antibody response can be generated upon immunization. Such mice are described in, for example, U.S. Pat. Nos. 5,476,996 and 5,698,767.

### Nucleic Acid Molecules of the Disclosure

The present disclosure also relates to nucleic acid molecules that encode antibodies disclosed herein. These sequences include, but are not limited to, those nucleic acid molecules set forth in FIGS. 1A, 1C, 1E, 1G, 1I, 1K, 2A, 2C, 2E, 2G, 2I, and 2K. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by any suitable techniques, including alkaline/SDS treatment, CsCl banding, column chromatography, agarose gel electrophoresis and others. A nucleic acid of the disclosure can be, for example, DNA or RNA and may or may not contain intronic sequences. Typically, the nucleic acid is a cDNA molecule.

Nucleic acids of the disclosure can be obtained using any suitable molecular biology techniques. For antibodies expressed by hybridomas, cDNAs encoding the light and heavy chains of the antibody made by the hybridoma can be obtained by PCR amplification or cDNA cloning techniques. For antibodies obtained from an immunoglobulin gene library (e.g., using phage display techniques), nucleic acid encoding the antibody can be recovered from the library.

The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g., Kabat et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. The IgG1 constant region sequence can be any of the various alleles or allotypes known to occur among different individuals, such as Gm(1), Gm(2), Gm(3), and Gm(17). These allotypes represent naturally occurring amino acid substitution in the IgG1 constant regions. For a Fab fragment heavy chain gene, the V_{H}-encoding DNA can be operatively linked to another DNA molecule encoding only the heavy chain CH1 constant region.

The isolated DNA encoding the V_{L} region can be converted to a full-length light chain gene (as well as a Fab light chain gene) by operatively linking the V_{L}-encoding DNA to another DNA molecule encoding the light chain constant region, CL. The sequences of human light chain constant region genes are known in the art (see e.g., Kabat et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

To create a scFv gene, the V_{H}- and V_{L}-encoding DNA fragments are operatively linked to another fragment encoding a flexible linker, e.g., encoding the amino acid sequence (Gly₄ -Ser)₃, such that the V_{H} and V_{L} sequences can be expressed as a contiguous single-chain protein, with the V_{L} and V_{H} regions joined by the flexible linker (see e.g., Bird et al., Science 242:423-426 (1988); Huston et al., Proc. Natl. Acad. Sci. USA 85:5879-5883 (1988); and McCafferty et al., Nature 348:552-554 (1990)).

### Nucleic Acid Variants

Nucleic acid molecules of the disclosure are not limited to the sequences disclosed herein, but also include variants thereof. Nucleic acid variants within the disclosure may be described by reference to their physical properties in hybridization. For example, the skilled worker will recognize that DNA can be used to identify its complement and, since DNA is double stranded, its equivalent or homolog, using nucleic acid hybridization techniques. It also will be recognized that hybridization can occur with less than 100% complementarity. However, given appropriate choice of conditions, hybridization techniques can be used to differentiate among DNA sequences based on their structural relatedness to a particular probe. For guidance regarding such conditions see, Sambrook and Russell, Molecular Cloning, A Laboratory Approach, Cold Spring Harbor Press, Cold Spring Harbor, N.Y. (2001) and Ausubel et al., Current Protocols in Molecular Biology, John Wiley & Sons, NY (2002).

Structural similarity between two polynucleotide sequences can be expressed as a function of “stringency” of the conditions under which the two sequences will hybridize with one another. As used herein, the term “stringency” refers to the extent that the conditions disfavor hybridization. Stringent conditions strongly disfavor hybridization, and only the most structurally related molecules will hybridize to one another under such conditions. Conversely, non-stringent conditions favor hybridization of molecules displaying a lesser degree of structural relatedness. Hybridization stringency, therefore, directly correlates with the structural relationships of two nucleic acid sequences. The following relationships are useful in correlating hybridization and relatedness (where Tₘ is the melting temperature of a nucleic acid duplex):
a. Tₘ=69.3+0.41(G+C)%
b. The Tₘ of a duplex DNA decreases by 1° C. with every increase of 1% in the number of mismatched base pairs.
c. (Tₘ)_{μ2}−(Tₘ)_{μ1}=18.5 log₁₀μ2/μ1
   where μ1 and μ2 are the ionic strengths of two solutions.

Hybridization stringency is a function of many factors, including overall DNA concentration, ionic strength, temperature, probe size and the presence of agents which disrupt hydrogen bonding. Factors promoting hybridization include high DNA concentrations, high ionic strengths, low temperatures, longer probe size and the absence of agents that disrupt hydrogen bonding. Hybridization typically is performed in two phases: the “binding” phase and the “washing” phase.

First, in the binding phase, the probe is bound to the target under conditions favoring hybridization. Stringency is usually controlled at this stage by altering the temperature. For high stringency, the temperature is usually between 65° C. and 70° C., unless short (<20 nucleotides) oligonucleotide probes are used. A representative hybridization solution comprises 6×SSC, 0.5% SDS, 5× Denhardt's solution and 100 μg of nonspecific carrier DNA. See Ausubel et al., Current Protocols in Molecular Biology, John Wiley & Sons, NY (2002). Of course, many different, yet functionally equivalent, buffer conditions are known. Where the degree of relatedness is lower, a lower temperature may be chosen. Low stringency binding temperatures are between about 25° C. and 40° C. Medium stringency is between at least about 40° C. to less than about 65° C. High stringency is at least about 65° C.

Second, the excess probe is removed by washing. It is at this phase that more stringent conditions usually are applied. Hence, it is this “washing” stage that is most important in determining relatedness via hybridization. Washing solutions typically contain lower salt concentrations. One exemplary medium stringency solution contains 2×SSC and 0.1% SDS. A high stringency wash solution contains the equivalent (in ionic strength) of less than about 0.2×SSC, with a preferred stringent solution containing about 0.1×SSC. The temperatures associated with various stringencies are the same as discussed above for “binding.” The washing solution also typically is replaced a number of times during washing. For example, typical high stringency washing conditions comprise washing twice for 30 minutes at 55° C., and three times for 15 minutes at 60° C.

Accordingly, the present disclosure includes nucleic acid molecules that hybridize to the DNA molecules as described herein under high stringency binding and washing conditions, where such nucleic molecules encode an antibody or functional fragment thereof having properties as described herein. Preferred molecules (from an mRNA perspective) are those that have at least 75% or 80% (preferably at least 85%, more preferably at least 90% and most preferably at least 95%) sequence identity with one of the DNA molecules described herein.

Yet another class of nucleic acid variants within the scope of the present disclosure may be described with reference to the product they encode. These functionally equivalent genes are characterized by the fact that they encode the same peptide sequences disclosed herein (e.g. in FIGS. 1 and 2) due to the degeneracy of the genetic code.

It is recognized that variants of DNA molecules provided herein can be constructed in several different ways. For example, they may be constructed as completely synthetic DNAs. Methods of efficiently synthesizing oligonucleotides in the range of 20 to about 150 nucleotides are widely available. See, e.g. Ausubel et al., Current Protocols in Molecular Biology, John Wiley & Sons, NY (2002). Overlapping oligonucleotides may be synthesized and assembled in a fashion first reported by Khorana et al., J. Mol. Biol. 72:209-217 (1971). Synthetic DNAs preferably are designed with convenient restriction sites engineered at the 5′ and 3′ ends of the gene to facilitate cloning into an appropriate vector.

As indicated, a method of generating variants is to start with one of the DNAs disclosed herein and then to conduct site-directed mutagenesis. See Ausubel et al., Current Protocols in Molecular Biology, John Wiley & Sons, NY (2002). In a typical method, a target DNA is cloned into a single-stranded DNA bacteriophage vehicle. Single-stranded DNA is isolated and hybridized with an oligonucleotide containing the desired nucleotide alteration(s). The complementary strand is synthesized and the double stranded phage is introduced into a host. Some of the resulting progeny will contain the desired mutant, which can be confirmed using DNA sequencing. In addition, various methods are available that increase the probability that the progeny phage will be the desired mutant. These methods are well known to those in the field and kits are commercially available for generating such mutants.

### Recombinant Nucleic Acid Constructs and Expression

The present disclosure further provides recombinant DNA constructs comprising one or more of the nucleotide sequences of the present disclosure. The recombinant constructs of the present disclosure are used in connection with a vector, such as a plasmid, phagemid, phage or viral vector, into which a DNA molecule encoding an antibody of the disclosure is inserted.

The encoded gene may be produced by techniques described in Sambrook and Russell, Molecular Cloning, A Laboratory Approach, Cold Spring Harbor Press, Cold Spring Harbor, N.Y. (2001), and Ausubel et al., Current Protocols in Molecular Biology, John Wiley & Sons, NY (2002). Alternatively, the DNA sequences may be chemically synthesized using, for example, synthesizers. See, for example, the techniques described in Oligonucleotide Synthesis (1984, Gait, ed., IRL Press, Oxford). For example, to express the antibodies of the disclosure, or antibody fragments thereof, DNAs encoding partial or full-length light and heavy chains, can be obtained by standard molecular biology techniques (e.g., PCR amplification or cDNA cloning using a hybridoma or phage that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by any suitable methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype and subclass by inserting them into expression vectors already encoding heavy chain constant and light chain constant regions of the desired isotype and subclass such that the V_{H} segment is operatively linked to the C_{H} segment(s) within the vector and the V_{K} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the disclosure typically carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SR promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors of the disclosure may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr- host cells with methotrexate selection/amplification) and the neo gene (for G418 selection).

For expression of the light and heavy chains, the expression vector(s) encoding the heavy and light chains is transfected into a host cell by any suitable techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the antibodies of the disclosure in either prokaryotic or eukaryotic host cells, expression of antibodies in eukaryotic cells, and typically mammalian host cells, is most typical.

The present disclosure further provides host cells containing at least one of the DNAs disclosed herein. The host cell can be virtually any cell for which expression vectors are available. It may be, for example, a higher eukaryotic host cell, such as a mammalian cell, a lower eukaryotic host cell, such as a yeast cell, and may be a prokaryotic cell, such as a bacterial cell. Introduction of the recombinant construct into the host cell can be effected by calcium phosphate transfection, DEAE, dextran mediated transfection, electroporation or phage infection.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus.

Bacterial vectors may be, for example, bacteriophage-, plasmid- or phagemid-based. These vectors can contain a selectable marker and bacterial origin of replication derived from commercially available plasmids typically containing elements of the well known cloning vector pBR322 (ATCC 37017). Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

In bacterial systems, a number of expression vectors may be advantageously selected depending upon the use intended for the protein being expressed. For example, when a large quantity of such a protein is to be produced, for the generation of antibodies or to screen peptide libraries, for example, vectors which direct the expression of high levels of fusion protein products that are readily purified may be desirable.

Mammalian host cells for expressing the recombinant antibodies of the disclosure include, for example, Chinese Hamster Ovary (CHO) cells (including dhfr- CHO cells, described in Urlaub and Chasin, Proc. Natl. Acad. Sci. USA 77:4216-4220 (1980), used with a DHFR selectable marker, e.g., as described in Kaufman and Sharp, J. Mol. Biol. 159:601-621 (1982), NS0 myeloma cells, COS cells and Sp2 cells. In particular, for use with NS0 myeloma or CHO cells, another expression system is the GS (glutamine synthetase) gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using any suitable protein purification methods.

### Immunoconjugates

In another aspect, the present disclosure features an OPN antibody, or a fragment thereof, conjugated to a therapeutic moiety, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Other examples of therapeutic cytotoxins that can be conjugated to an antibody, or antigen binding portion thereof, of the disclosure include duocarmycins, calicheamicins, maytansines and auristatins, and derivatives thereof. An example of a calicheamicin antibody conjugate is commercially available (Mylotarg™; Wyeth-Ayerst).

Cytoxins can be conjugated to antibodies of the disclosure or antigen binding portions thereof using various linker technologies. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

For further discussion of types of cytotoxins, linkers and methods for conjugating therapeutic agents to antibodies, see also Saito et al., Adv. Drug Deliv. Rev. 55:199-215 (2003); Trail, et al., Cancer Immunol. Immunother. 52:328-337 (2003); Payne, Cancer Cell 3:207-212 (2003); Allen, Nat. Rev. Cancer 2:750-763 (2002); Pastan, I. and Kreitman, Curr. Opin. Investig. Drugs 3:1089-1091 (2002); Senter, P. D. and Springer, C. J. (2001) Adv. Drug Deliv. Rev. 53:247-264.

Antibodies or antigen binding portions thereof of the present disclosure also can be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine¹³¹, indium¹¹¹, yttrium⁹⁰ and lutetium¹⁷⁷. Methods for preparing radioimmunoconjugates are established in the art. Examples of radioimmunoconjugates are commercially available, including Zevalin™ (IDEC Pharmaceuticals) and Bexxar™ (Corixa Pharmaceuticals), and similar methods can be used to prepare radioimmunoconjugates using the antibodies of the disclosure.

The antibody conjugates of the disclosure can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-γ; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are known. See, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-256 (Alan R. Liss, Inc. 1985); Hellstrom et al., “Antibodies For Drug Delivery”, in Controlled Drug Delivery (2nd Ed.), Robinson et al. (eds.), pp. 623-653 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-316 (Academic Press 1985), and Thorpe et al., Immunol. Rev., 62:119-158 (1982).

### Bispecific Molecules

In another aspect, the present disclosure features bispecific molecules comprising an OPN antibody, or an antigen-binding portion thereof, of the present disclosure. An antibody of the disclosure, or antigen-binding portion thereof, can be derivatized or linked to another functional molecule, e.g., another peptide or protein (e.g., another antibody or ligand for a receptor) to generate a bispecific molecule that binds to at least two different binding sites or target molecules. The antibodies of the disclosure may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule of the disclosure, an antibody of the disclosure can be functionally linked (e.g., by chemical coupling, genetic fusion, noncovalent association or otherwise) to one or more other binding molecules, such as another antibody, antibody fragment, peptide or binding mimetic, such that a bispecific molecule results.

Accordingly, the present disclosure includes bispecific molecules comprising at least one first binding specificity for OPN and a second binding specificity for a second target epitope. In a particular aspect of the disclosure, the second target epitope is an Fc receptor, e.g., human FcγRI (CD64) or a human Fcγ receptor (CD89). Therefore, the disclosure includes bispecific molecules capable of binding both to FcγR or FcγR expressing effector cells (e.g., monocytes, macrophages or polymorphonuclear cells (PMNs)), and to OPN. These bispecific molecules target OPN to effector cell and trigger Fc receptor-mediated effector cell activities, such as phagocytosis of an OPN expressing cell, antibody dependent cell-mediated cytotoxicity (ADCC), cytokine release, or generation of superoxide anion.

In an aspect of the disclosure in which the bispecific molecule is multispecific, the molecule can further include a third binding specificity, in addition to an anti-Fc binding specificity and an OPN binding specificity. In one case, the third binding specificity is an anti-enhancement factor (EF) portion, e.g., a molecule which binds to a surface protein involved in cytotoxic activity and thereby increases the immune response against the target cell. The “anti-enhancement factor portion” can be an antibody, functional antibody fragment or a ligand that binds to a given molecule, e.g., an antigen or a receptor, and thereby results in an enhancement of the effect of the binding determinants for the Fc receptor or target cell antigen. The “anti-enhancement factor portion” can bind an Fc receptor or a target cell antigen. Alternatively, the anti-enhancement factor portion can bind to an entity that is different from the entity to which the first and second binding specificities bind. For example, the anti-enhancement factor portion can bind a cytotoxic T-cell (e.g. via CD2, CD3, CD8, CD28, CD4, CD40, ICAM-1 or other immune cell that results in an increased immune response against the target cell).

In one case, the bispecific molecules of the disclosure comprise as a binding specificity at least one antibody, or an antibody fragment thereof, including, e.g., an Fab, Fab′, F(ab′)₂, Fv, or a single chain Fv. The antibody may also be a light chain or heavy chain dimer, or any minimal fragment thereof such as a Fv or a single chain construct as described in U.S. Pat. No. 4,946,778.

In one case, the binding specificity for an Fcγ receptor is provided by a monoclonal antibody, the binding of which is not blocked by human immunoglobulin G (IgG). As used herein, the term “IgG receptor” refers to any of the eight γ-chain genes located on chromosome 1. These genes encode a total of twelve transmembrane or soluble receptor isoforms which are grouped into three Fcγ receptor classes: FcγRI (CD64), FcγRII(CD32), and FcγRIII (CD16). In one case, the Fcγ receptor is a human high affinity FcγRI. The human FcγRI is a 72 kDa molecule, which shows high affinity for monomeric IgG (10⁸ to 10⁹ M⁻¹).

The production and characterization of certain anti-Fcy monoclonal antibodies are described in PCT Publication WO 88/00052 and in U.S. Pat. No. 4,954,617. These antibodies bind to an epitope of FcγRI, FcγRII or FcγRIII at a site which is distinct from the Fcγ binding site of the receptor and, thus, their binding is not blocked substantially by physiological levels of IgG. Specific anti-FcγRI antibodies useful in this disclosure are mAb 22, mAb 32, mAb 44, mAb 62 and mAb 197. The hybridoma producing mAb 32 is available from the American Type Culture Collection, ATCC Accession No. HB9469. In other cases the anti-Fcγ receptor antibody is a humanized form of monoclonal antibody 22 (H22). The production and characterization of the H22 antibody is described in Graziano et al. J. Immunol 155(10):4996-5002 (1995) and PCT Publication WO 94/10332. The H22 antibody producing cell line was deposited at the American Type Culture Collection under the designation HA022CL1 and has the Accession No. CRL 11177.

In still other cases, the binding specificity for an Fc receptor is provided by an antibody that binds to a human IgA receptor, e.g., an Fc-alpha receptor (FcαRI (CD89)), the binding of which is typically not blocked by human immunoglobulin A (IgA). The term “IgA receptor” is intended to include the gene product of one α-gene (FcαRI) located on chromosome 19. This gene is known to encode several alternatively spliced transmembrane isoforms of 55 to 110 kDa. FcαRI (CD89) is constitutively expressed on monocytes/macrophages, eosinophilic and neutrophilic granulocytes, but not on non-effector cell populations. FcαRI has medium affinity 5×10⁷ M⁻¹) for both IgA1 and IgA2, which is increased upon exposure to cytokines such as G-CSF or GM-CSF (Morton et al., Critical Reviews in Immunology 16:423-440 (1996)). Four FcαRI-specific monoclonal antibodies, identified as A3, A59, A62 and A77, which bind FcαRI outside the IgA ligand binding domain, have been described (Monteiro et al., J. Immunol. 148:1764 (1992)).

FcαRI and FcγRI are illustrative trigger receptors for use in the bispecific molecules of the disclosure because they are (1) expressed primarily on immune effector cells, e.g., monocytes, PMNs, macrophages and dendritic cells; (2) expressed at high levels (e.g., 5,000 to 100,000 per cell); (3) mediators of cytotoxic activities (e.g., ADCC, phagocytosis); (4) mediate enhanced antigen presentation of antigens, including self-antigens, targeted to them.

While human monoclonal antibodies are preferred, other antibodies which can be employed in the bispecific molecules of the disclosure are murine, chimeric and humanized monoclonal antibodies.

The bispecific molecules of the present disclosure can be prepared by conjugating the constituent binding specificities, e.g., the anti-FcR and anti-OPN binding specificities, using any suitable methods. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-1-carboxylate (sulfo-SMCC) (see e.g., Karpovsky et al., J. Exp. Med. 160:1686 (1984); Liu et al., Proc. Natl. Acad. Sci. USA 82:8648 (1985)). Other methods include those described in Paulus (1985) Behring Ins. Mitt. No. 78, 118-132; Brennan et al., Science 229:81-83 (1985)), and Glennie et al., J. Immunol. 139:2367-2375 (1987)). Suitable conjugating agents include SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In one case, the hinge region is modified to contain an odd number of sulfhydryl residues, such as one residue, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand×Fab fusion protein. A bispecific molecule of the disclosure can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986). The radioactive isotope can be detected by such means as the use of a γ counter or a scintillation counter or by autoradiography.

### Therapeutic Methods

Therapeutic methods involve administering to a subject in need of treatment a therapeutically effective amount, or “effective amount”, of an antibody, or antigen-binding portion, contemplated by the present disclosure. As used herein, a “therapeutically effective”, or “effective”, amount refers to an amount of an antibody or portion thereof that is of sufficient quantity to result in a decrease in severity of disease symptoms, an increase in frequency and duration of disease symptom-free periods, or a prevention of impairment or disability due to the disease affliction—either as a single dose or according to a multiple dose regimen, alone or in combination with other agents. One of ordinary skill in the art would be able to determine such amounts based on such factors as the subject's size, the severity of the subject's symptoms, and the particular composition or route of administration selected. The subject may be a human or non-human animal (e.g., rabbit, rat, mouse, monkey or other lower-order primate).

An antibody or antigen-binding portion of the disclosure might be co-administered with known medicaments, and in some instances the antibody might itself be modified. For example, an antibody could be conjugated to an immunotoxin or radioisotope to potentially further increase efficacy. Regarding co-administration with additional therapeutic agents, such agents can include a cytotoxic agent, a radiotoxic agent or an immunosuppressive agent. The antibody can be linked to the agent (as an immunocomplex) or can be administered separately from the agent. In the latter case (separate administration), the antibody can be administered before, after or concurrently with the agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil, and cyclophosphamide hydroxyurea which, by themselves, are only effective at levels which are toxic or subtoxic to a patient. Cisplatin can be intravenously administered as a 100 mg dose once every four weeks and adriamycin is intravenously administered as a 60 to 75 mg dose once every 21 days. Co-administration of the OPN antibodies, or antigen binding fragments thereof, of the present disclosure with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody.

The antibodies and antigen-binding portions disclosed herein can be used as a therapeutic or a diagnostic tool in a variety of situations where OPN is undesirably expressed or found. Given the expression of OPN by various tumor cells, and the role that OPN plays in tumor metastasis, disorders and conditions particularly suitable for treatment with an antibody or antigen-binding portion of the present disclosure include abnormal cell growth, for example, mesothelioma, hepatobilliary (hepatic and billiary duct), a primary or secondary CNS tumor, a primary or secondary brain tumor, lung cancer (NSCLC and SCLC), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non-Hodgkin's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

To treat any of the foregoing disorders, pharmaceutical compositions for use in accordance with the present disclosure may be formulated in a conventional manner using one or more pharmaceutically acceptable carriers or excipients. An antibody or antigen-binding portion of the disclosure can be administered by any suitable means, which can vary, depending on the type of disorder being treated. Possible administration routes include parenteral (e.g., intramuscular, intravenous, intra-arterial, intraperitoneal, or subcutaneous), intrapulmonary and intranasal, and, if desired for local immunosuppressive treatment, intralesional administration. In addition, an antibody of the disclosure might be administered by pulse infusion, with, e.g., declining doses of the antibody. Preferably, the dosing is given by injections, most preferably intravenous or subcutaneous injections, depending in part on whether the administration is brief or chronic. The amount to be administered will depend on a variety of factors such as the clinical symptoms, weight of the individual, whether other drugs are administered. The skilled artisan will recognize that the route of administration will vary depending on the disorder or condition to be treated.

Determining a therapeutically effective amount of an antibody or antigen-binding portion according to the present disclosure will largely depend on particular patient characteristics, route of administration, and the nature of the disorder being treated. General guidance can be found, for example, in the publications of the International Conference on Harmonization and in Remington's Pharmaceutical Sciences, chapters 27 and 28, pp. 484-528 (18th ed., Alfonso R. Gennaro, Ed., Easton, Pa.: Mack Pub. Co., 1990). More specifically, determining a therapeutically effective amount will depend on such factors as toxicity and efficacy of the medicament. Toxicity may be determined using methods well known in the art and found in the foregoing references. Efficacy may be determined utilizing the same guidance in conjunction with the methods described below in the Examples.

For administration of the antibody, the dosage can range from about 0.0001 to 100 mg/kg, and more usually 0.01 to 20 mg/kg, of the host body weight. For example dosages can be 0.3 mg/kg body weight, 1 mg/kg body weight, 3 mg/kg body weight, 5 mg/kg body weight, 10 mg/kg body weight, 15 mg/kg body weight, 20 mg/kg body weight, or within the range of 1 to 20 mg/kg. An exemplary treatment regime entails administration once per week, once every two weeks, once every three weeks, once every four weeks, once per month, once every 3 months or once every three to 6 months. Dosage regimens for an anti-OPN antibody or antigen binding portion thereof of the disclosure include, for example, 1 mg/kg body weight, 3 mg/kg body weight, 5 mg/kg body weight, 10 mg/kg body weight, 15 mg/kg body weight, or 20 mg/kg body weight via intravenous administration, with the antibody being given using one of the following dosing schedules: (i) every four weeks for six dosages, then every three months; (ii) every three weeks; (iii) 1-20 mg/kg body weight once followed by 1-20 mg/kg body weight every three weeks.

### Diagnostic Methods

OPN is highly expressed in various tumor cells; thus, an anti-OPN antibody of the disclosure may be employed in order to image or visualize a site of possible OPN in a patient. In this regard, an antibody can be detectably labeled, through the use of radioisotopes, affinity labels (such as biotin, avidin, etc.) fluorescent labels, paramagnetic atoms, etc. Procedures for accomplishing such labeling are well known to the art. Clinical application of antibodies in diagnostic imaging are reviewed by Grossman, Urol. Clin. North Amer. 13:465-474 (1986), Unger et al., Invest. Radiol. 20:693-700 (1985), and Khaw et al., Science 209:295-297 (1980).

The detection of foci of such detectably labeled antibodies might be indicative of certain types of cancer, for example. In one embodiment, this examination is done by removing samples of tissue or blood and incubating such samples in the presence of the detectably labeled antibodies. In a preferred embodiment, this technique is done in a non-invasive manner through the use of magnetic imaging, fluorography, etc. Such a diagnostic test may be employed in monitoring the success of treatment of diseases, where presence or absence of a target OPN-positive cell is a relevant indicator.

### Therapeutic and Diagnostic Compositions

The antibodies and antigen-binding portions of the present disclosure can be formulated according to known methods to prepare pharmaceutically useful compositions, wherein at least one antibody of the present disclosure (including any antigen-binding portion thereof) is combined in a mixture with a pharmaceutically acceptable carrier. Suitable carriers and their formulation are described, for example, in Remington's Pharmaceutical Sciences (18th ed., Alfonso R. Gennaro, Ed., Easton, Pa.: Mack Pub. Co., 1990). In order to form a pharmaceutically acceptable composition suitable for effective administration, such compositions will contain an effective amount of one or more of the antibodies of the present disclosure, together with a suitable amount of a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Typically, the carrier is suitable for intravenous, intramuscular, subcutaneous, parenteral, spinal or epidermal administration (e.g., by injection or infusion). Depending on the route of administration, the active compound, i.e., antibody, antigen-binding portion thereof, immunoconjugate, or bispecific molecule, may be coated in a material to protect the compound from the action of acids and other natural conditions that may inactivate the compound.

In certain embodiments, the antibodies of the present disclosure may be present in a neutral form (including zwitter ionic forms) or as a positively or negatively-charged species. In some cases, the antibodies may be complexed with a counterion to form a pharmaceutically acceptable salt. Thus, the pharmaceutical compounds of the disclosure may include one or more pharmaceutically acceptable salts.

A “pharmaceutically acceptable salt” refers to a salt that retains the desired biological activity of the parent compound (e.g. antibody) and does not impart undesired toxicological effects (see e.g., Berge, S. M., et al. (1977) J. Pharm. Sci. 66:1-19). For example, the term “pharmaceutically acceptable salt” includes a complex comprising one or more antibodies and one or more counterions, where the counterions are derived from pharmaceutically acceptable inorganic and organic acids and bases.

Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Furthermore, pharmaceutically acceptable inorganic bases include metallic ions. Metallic ions include, but are not limited to, appropriate alkali metal salts, alkaline earth metal salts and other physiological acceptable metal ions. Salts derived from inorganic bases include aluminum, ammonium, calcium, cobalt, nickel, molybdenum, vanadium, manganese, chromium, selenium, tin, copper, ferric, ferrous, lithium, magnesium, manganic salts, manganous, potassium, rubidium, sodium, and zinc, and in their usual valences.

Pharmaceutically acceptable acid addition salts of the antibodies of the present disclosure can be prepared from the following acids, including, without limitation formic, acetic, acetamidobenzoic, adipic, ascorbic, boric, propionic, benzoic, camphoric, carbonic, cyclamic, dehydrocholic, malonic, edetic, ethylsulfuric, fendizoic, metaphosphoric, succinic, glycolic, gluconic, lactic, malic, tartaric, tannic, citric, nitric, ascorbic, glucuronic, maleic, folic, fumaric, propionic, pyruvic, aspartic, glutamic, benzoic, hydrochloric, hydrobromic, hydroiodic, lysine, isocitric, trifluoroacetic, pamoic, propionic, anthranilic, mesylic, orotic, oxalic, oxalacetic, oleic, stearic, salicylic, aminosalicylic, silicate, p-hydroxybenzoic, nicotinic, phenylacetic, mandelic, embonic, sulfonic, methanesulfonic, phosphoric, phosphonic, ethanesulfonic, ethanedisulfonic, ammonium, benzenesulfonic, pantothenic, naphthalenesulfonic, toluenesulfonic, 2-hydroxyethanesulfonic, sulfanilic, sulfuric, nitric, nitrous, sulfuric acid monomethyl ester, cyclohexylaminosulfonic, β-hydroxybutyric, glycine, glycylglycine, glutamic, cacodylate, diaminohexanoic, camphorsulfonic, gluconic, thiocyanic, oxoglutaric, pyridoxal 5-phosphate, chlorophenoxyacetic, undecanoic, N-acetyl-L-aspartic, galactaric and galacturonic acids.

Pharmaceutically acceptable organic bases include trimethylamine, diethylamine, N, N′-dibenzylethylenediamine, chloroprocaine, choline, dibenzylamine, diethanolamine, ethylenediamine, meglumine(N-methylglucamine), procaine, cyclic amines, quaternary ammonium cations, arginine, betaine, caffeine, clemizole, 2-ethylaminoethanol, 2-diethylaminoethanol, 2-dimethylaminoethanol, ethanediamine, butylamine, ethanolamine, ethylenediamine, N-ethylmorpholine, N-ethylpiperidine, ethylglucamine, glucamine, glucosamine, histidine, hydrabamine, imidazole, isopropylamine, methylglucamine, morpholine, piperazine, pyridine, pyridoxine, neodymium, piperidine, polyamine resins, procaine, purines, theobromine, triethylamine, tripropylamine, triethanolamine, tromethamine, methylamine, taurine, cholate, 6-amino-2-methyl-2-heptanol, 2-amino-2-methyl-1,3-propanediol, 2-amino-2-methyl-1-propanol, aliphatic mono- and dicarboxylic acids, phenyl-substituted alkanoic acids, hydroxy alkanoic acids, aromatic acids, aliphatic and aromatic sulfonic acids, strontium, tricine, hydrazine, phenylcyclohexylamine, 2-(N-morpholino)ethanesulfonic acid, bis(2-hydroxyethyl)amino-tris(hydroxymethyl)methane, N-(2-acetamido)-2-aminoethanesulfonic acid, 1,4-piperazinediethanesulfonic acid, 3-morpholino-2-hydroxypropanesulfonic acid, 1,3-bis[tris(hydroxymethyl)methylamino]propane, 4-morpholinepropanesulfonic acid, 4-(2-hydroxyethyl)piperazine-1-ethanesulfonic acid, 2-[(2-hydroxy-1,1-bis(hydroxymethyl)ethyl)amino]ethanesulfonic acid, N,N-bis(2-hydroxyethyl)-2-aminoethanesulfonic acid, 4-(N-morpholino)butanesulfonic acid, 3-(N,N-bis[2-hydroxyethyl]amino)-2-hydroxypropanesulfonic acid, 2-hydroxy-3-[tris(hydroxymethyl)methylamino]-1-propanesulfonic acid, 4-(2-hydroxyethyl)piperazine-1-(2-hydroxypropanesulfonic acid), piperazine-1,4-bis(2-hydroxypropanesulfonic acid)dihydrate, 4-(2-hydroxyethyl)-1-piperazinepropanesulfonic acid, N,N-bis(2-hydroxyethyl)glycine, N-(2-hydroxyethyl)piperazine-N′-(4-butanesulfonic acid), N-[tris(hydroxymethyl)methyl]-3-aminopropanesulfonic acid, N-tris(Hydroxymethyl)methyl-4-aminobutanesulfonic acid, N-(1,1-dimethyl-2-hydroxyethyl)-3-amino-2-hydroxypropanesulfonic acid, 2-(cyclohexylamino)ethanesulfonic acid, 3-(cyclohexylamino)-2-hydroxy-1-propanesulfonic acid, 3-(cyclohexylamino)-1-propanesulfonic acid, N-(2-acetamido)iminodiacetic acid, 4-(cyclohexylamino)-1-butanesulfonic acid, N-[tris(hydroxymethyl)methyl]glycine, 2-amino-2-(hydroxymethyl)-1,3-propanediol, and trometamol.

A pharmaceutical composition of the disclosure also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include, but are not limited to, vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the disclosure are dictated by and directly dependent on (a) the unique characteristics of the active compound and the particular therapeutic effect to be achieved, and (b) the limitations inherent in the art of compounding such an active compound for the treatment of sensitivity in individuals.

Preparations may be suitably formulated to provide controlled-release of the active compound. Controlled-release preparations may be achieved through the use of polymers to complex or absorb an anti-OPN antibody. The controlled delivery may be exercised by selecting appropriate macromolecules (for example polyesters, polyamino acids, polyvinyl, pyrrolidone, ethylenevinyl-acetate, methylcellulose, carboxymethylcellulose, or protamine, sulfate) and the concentration of macromolecules as well as the methods of incorporation in order to control release. Another possible method to control the duration of action by controlled release preparations is to incorporate an anti-OPN antibody into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatine-microcapsules and poly(methylmethacylate) microcapsules, respectively, or in colloidal drug delivery systems, for example, liposomes, albumin microspheres, microemulsions, nanoparticles, and nanocapsules or in macroemulsions. Such techniques are disclosed in Remington's Pharmaceutical Sciences (18th ed., Alfonso R. Gennaro, Ed., Easton, Pa.: Mack Pub. Co., 1990).

Antibody preparations may be formulated for parenteral administration by injection, e.g., by bolus injection or continuous infusion. Formulations for injection may be presented in unit dosage form, e.g., in ampules, or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compositions may, if desired, be presented in a pack or dispenser device, which may contain one or more unit dosage forms containing the active ingredient. The pack may, for example, comprise metal or plastic foil, such as a blister pack. The pack or dispenser device may be accompanied by instructions for administration.

The present disclosure is further illustrated by the following examples which should not be construed as further limiting. The contents of all figures and all references, patents and published patent applications cited throughout this disclosure are expressly incorporated herein by reference in their entirety.

### EXAMPLES

As used in the Examples below, the following abbreviations have the following meanings, unless indicated otherwise, are readily available from commercial suppliers: PBS: phosphate buffered saline, pH 7.4; IPTG: Isopropyl-β-D-thiogalactopyranoside; HSA: human serum albumin;

### Example 1

### Antibody Generation from HuCAL® Libraries

For the generation of therapeutic antibodies against OPN, selections with the MorphoSys HuCAL GOLD® phagemid library were carried out. The phagemid library is based on the HuCAL® concept (Knappik et al., J. Mol. Biol. 296:57 (2000)) and employs the CysDisplay™ technology for displaying the Fab on the phage surface (Löhning, WO 01/05950). HuCAL GOLD® antibody-phage of different frameworks were either combined to form one pool (VH1-6) or were divided into sub-pools (e.g. VH1/5, VH2/4/6, VH3) and subsequently these sub-pools were individually subjected to selection rounds on antigen as described below. Phage for the 1st round of pannings were prepared by Hyperphage (M13KO7ΔpIII, obtained from Progen, Heidelberg, Germany).

### Solid Phase Panning Against OPN

Solid phase panning was performed using recombinant human OPN (R&D Systems #1433-OP/CF, carrier free), recombinant mouse OPN (R&D Systems #441-OP/CF, carrier free), or SPP1 peptides comprising functional domains of human OPN or mouse OPN (27 aa). Different antigens were used for either 3 rounds with or without alternating between human and mouse OPN as well as strategies including alternating of human/mouse OPN antigen with SPP1 peptides. For pannings using OPN proteins, different buffer conditions were used. Antigen was coated either in PBS or PBS supplemented with Ca²⁺ and Mg²⁺ (100 mg/L CaCl₂; 100 mg/L MgCl₂).

A final concentration of 50 μg/mL diluted in PBS or PBS (supplemented with Ca²⁺ and Mg²⁺) of human/mouse OPN was used for coating of an appropriate number of wells on Maxisorp™ plates (F96 Maxisorp™, 442402, Nunc, Rochester, N.Y.) for the first and second round of pannings. For the third round, 25 μg/mL of human and 10 μg/mL of mouse OPN were used for coating. Respective plates were then incubated overnight at 4° C. On the next day the wells were washed twice with PBS and then blocked with MPBST (PBS, 0.05% Tween20 (Sigma, St. Louis, Mo., USA), 5% milk powder) for 2 hours at room temperature. 100 μL of phage from original HuCAL GOLD® subpools (VH1-6, VH1/5 and VH3, prepared with hyperphage) were used. Phages were pre-blocked in a PBS solution containing 2.5% milk powder, 2.5% BSA and 0.05% Tween20. The pre-blocking of phage was performed in 2 mL reaction tubes for 2 hours at room temperature on a rotator.

For the selection process, the antigen solution was removed from the Maxisorp™ plate and the wells were washed three times with PBS. The pre-blocked phage were added to the corresponding wells and the plate was incubated for 2 hours at room temperature on a microplate shaker. The phage solution was then removed and the wells were washed several times (stringency depending on the panning strategy and selection round) with PBST (PBS, 0.05% Tween20), followed by the same washing steps with PBS. The washing stringency was increased from round to round. PBS was removed after the last washing step before continuing with elution. For elution of specifically bound phage, 20 mM DTT in 10 mM Tris/HCl, pH 8.0 was added and the samples were incubated for 10 minutes at room temperature.

The eluates were used to infect log phase E. coli TG1 cultures. Infected E. coli were harvested by centrifugation and plated onto LB agar plates supplemented with 34 μg/mL chloramphenicol and 1% glucose. The agar plates were incubated overnight at 30° C. On the following day the colonies were scraped off and grown until reaching an OD600 nm of 0.5 to proceed to helper phage infection. Helper phage infection: TG1 cells were infected with the helper phage VCSM13 (multiplicity of infection of at least 20) at 37° C. The infected cells were harvested by centrifugation and resuspended in 2×YT medium containing 34 μg/mL chloramphenicol, 50 μg/mL kanamycin and 0.25 mM IPTG for induction of Fab expression. The cells were grown overnight and the produced phage were precipitated from the supernatant with polyethylene glycol (PEG)/NaCl and resuspended in PBS. Input and output titers were determined by spot titration.

### Solution Panning Against OPN

Solution panning was performed using recombinant human OPN, recombinant mouse OPN, or SPP1 peptides comprising functional domains of hOPN or mOPN (27aa). Different antigens were used for either 3 rounds with or without alternating between human and mouse OPN as well as strategies including alternating of h/m OPN antigen with SPP1 peptides. For pannings using OPN proteins, different buffer conditions were used. Antigen was used either in PBS or PBS supplemented with Ca²⁺ and Mg²⁺ (100 mg/L CaCl₂; 100 mg/L MgCl₂).

All tubes used for the selections were pre-blocked with ChemiBLOCKER (Chemicon, Temecula, Calif., USA). HuCAL GOLD® phage were blocked with ChemiBLOCKER (+0.05% Tween20) and pre-adsorbed twice on M-280 Streptavidin Dynabeads® (Dynal Biotech, Oslo, Norway). Pre-blocked phage and biotinylated OPN protein or peptide antigen were incubated in a 2 mL tube for 2 hours at room temperature on a rotator. For the first selection round, 100 nM of biotinylated antigen concentration was used for bead coupling. The second and third panning rounds were performed using 10 nM biotinylated antigens.

Pre-adsorbed Streptavidin Dynabeads® were added to the phage-antigen solution and incubated further for 10 minutes at room temperature on a rotator. A magnetic particle separator, MPC-E (Dynal Biotech, Oslo, Norway), was used to separate phage bound to the captured antigen. The beads were washed several times with PBST (PBS, 0.05% Tween 20), followed by several washing steps with PBS. The washing stringency was increased with every panning round. PBS was removed after the last washing step before continuing with elution. Elution and further steps were performed as described previously for the solid phase panning.

### Subcloning and Microexpression of Selected Fab Fragments

To facilitate rapid expression of soluble Fab, the Fab encoding inserts of the selected HuCAL GOLD® phage were subcloned via XbaI and EcoRI into the expression vector pMORPH®X9_MH. After transformation of the expression plasmids into E. coli TG1 F-cells chloramphenicol-resistant single clones were picked into the wells of a sterile 384-well microtiter plate pre-filled with 2×YT medium (supplemented with 34 μg/mL chloramphenicol and 1% glucose) and grown overnight at 37° C. These plates were regarded as master plates. Before storage of the master plates at −80° C., the E. coli TG1 F-cultures were inoculated into new, sterile 384-well microtiter plates pre-filled with 40 μL 2×YT medium supplemented with 34 μg/mL chloramphenicol and 0.1% glucose per well. The microtiter plates were incubated at 30° C. shaking at 400 rpm on a microplate shaker until the cultures were slightly turbid (˜2 to 4 hours) with an OD600 of ˜0.5. These plates were regarded as expression plates, and 10 μL 2×YT medium supplemented with 34 μg/mL chloramphenicol and 5 mM IPTG was added per well (end concentration 1 mM IPTG), the microtiter plates were sealed with a gas-permeable tape, and incubated overnight at 30° C. shaking at 400 rpm.

Generation of whole cell lysates (BEL extracts): To each well of the expression plates, 15 μL BEL buffer was added and incubated for 1 hour at 22° C. on a microtiter plate shaker (400 rpm). BEL buffer: 24.7 g/L boric acid, 18.7 g NaCl/L, 1.49 g EDTA/I, pH 8.0 supplemented with 2.5 mg/mL lysozyme.

### Expression and Purification of HuCAL-Fab Antibodies in E. Coli

Expression of Fab fragments encoded by pMORPHX9_FH in TG-1 F-cells was carried out in shaker flask cultures with 1 L of 2×TY medium supplemented with 34 μg/mL chloramphenicol. After induction with 0.5 mM IPTG, cells were grown at 30° C. for 20 hours. Whole cell lysis (Lysozyme) of cell pellets were prepared and Fab fragments isolated by HT-IMAC-purification. The apparent molecular weights were determined by size exclusion chromatography (SEC) with calibration standards. Concentrations were determined by UV-spectrophotometry.

### Example 2

### Screening of OPN Positive Clones

OPN positive clones were further identified by screening the clones generated in Example 1 for antigen binding using the ELISA assay methods as described below.

### Screening on Directly Coated OPN

Primary and secondary screening was performed using hOPN and mOPN protein as well as SSP1 peptides. hOPN was used for overnight coating of Maxisorp® microtiter plates at 4° C. at a concentration of 12.5 μg/mL (diluted in PBS), mOPN was coated at a concentration of 5 μg/mL.

After overnight incubation, coated plates were washed twice with PBST (PBS/0.05% Tween20) and blocked with 5% MPBST (5% milkpowder in PBST) for 1 hour at room temperature on a microplate shaker. The plates were washed twice with PBST before primary antibodies were added (crude extracts of microexpressed HuCAL® Fabs, purified HuCAL® Fabs, anti-OPN monoclonal control antibody AKm2A1, 1:200, Santa Cruz #SC-21742). The plates containing the primary antibodies were incubated for 1 hour at room temperature on a microplate shaker. The plates were washed twice with PBST and for the detection of HuCAL® Fabs the secondary antibody (Goat anti-human F(ab)₂—Fragment specific—AP labeled, Jackson Cat. No. 109-055-097) was added, diluted 1:5000 in 0.5% MPBST. The plate containing the secondary antibodies was incubated for 1 hour at room temperature on a microplate shaker. The wells were washed five times with TBST (TBS/0.05% Tween20), Attophos (AttoPhos Substrate Set, Roche, #11681982001) was added (diluted 1:10 in water) and fluorescence emission at 535 nm was recorded with excitation at 430 nm.

### Capture Screening Using Biotinylated OPN Protein

Maxisorp (Nunc, Rochester, N.Y., USA) 384 well plates were coated with 20 μL/well NeutrAvidin™ biotin binding protein (Pierce, Cat. No. #31000) at a final concentration of 10 μg/mL diluted in PBS, pH 7.4 by incubation over night at 4° C. and 450 rpm on a microplate shaker.

The following day, the plates were washed two times using TBST (TBS/0.05% Tween20) and blocked for 1 hour using 90 μL/well Superblock solution (Pierce, Cat. No. #37545). Blocked plates were washed three times using TBST followed by incubation for 2 hours of 10 μL/well biotinylated human or mouse OPN protein (2 μg/mL). Plates were washed three times using TBST followed by blocking for 1 hour by incubation of 90 mL/well 10% BSA in TBS. Plates were finally washed five times using TBST before 40 μL/well BEL extract was incubated for 1.5 hours at room temperature. Subsequently HuCAL® Fab fragments were allowed to bind to captured biotinylated antigen OPN. The plates were washed twice with PBST and for the detection of HuCAL® Fabs the secondary antibody (Goat anti-human F(ab)₂—Fragment specific—AP labeled, Jackson Cat. No. #109-055-097) was added, diluted 1:5000 in 0.5% MPBST. The plate containing the secondary antibodies was incubated for 1 hour at room temperature on a microplate shaker. The wells were washed five times with TBST, Attophos (AttoPhos Substrate Set, Roche, #11681982001) was added (diluted 1:10 in water) and fluorescence emission at 535 nm was recorded with excitation at 430 nm.

The EC₅₀ values (determined as described above) for twelve selected Fabs are shown below in Table 2.

TABLE 2
Summary of ELISA EC₅₀ Values for Twelve Selected Fabs

| | Protein ELISA | | | | Peptide ELISA | | | |
|---|---|---|---|---|---|---|---|---|
| | hOPN | | mOPN | | hOPN | | mOPN | |
| Fab | EC₅₀ | Std | EC₅₀ | Std | EC₅₀ | Std | EC₅₀ | Std |
| 6453 | 1.3 | 0.5 | 2.8 | 1.6 | no binding | | no binding | |
| 6454 | 1.5 | 0.8 | 393.0 | 179.6 | no binding | | no binding | |
| 6455 | 0.7 | 0.4 | n.d. | n.d. | no binding | | no binding | |
| 6989 | 1.8 | 2.4 | 3.7 | 3.5 | 0.7 | 0.4 | 0.5 | 0.4 |
| 6990 | 8.3 | 7.9 | 3.3 | 2.1 | 1.2 | 1.1 | 0.4 | 0.3 |
| 6991 | 3.4 | 3.3 | 327.2 | 214.1 | 0.3 | 0.1 | 49.0 | 37.00.5 |
| 6992 | 0.9 | 0.6 | 4.5 | 3.1 | 3.9 | 5.0 | 1.0 | 0.1 |
| 6993 | 1.2 | 0.8 | 0.9 | 0.7 | 0.4 | 0.3 | 0.3 | |
| 7201 | 10.5 | 8.1 | 15.0 | 14.1 | no binding | | no binding | |
| 7202 | 242.0 | 196.6 | 16.8 | 10.7 | no binding | | no binding | |
| 7203 | 1.7 | 2.0 | 12.2 | 9.8 | 1.2 | n.d. | 2.2 | 1.3 |

| | | | | | | |
|---|---|---|---|---|---|---|
| 7212 | 5.2 | 0.8 | 9.8 | 2.1 | no binding | no binding |

n.d. = not determined;
Std = standard deviation

The EC₅₀ values (determined as described above) for seven of the selected IgGs (seven selected Fabs were converted to full length human IgG2 antibodies as described in Example 4) are shown below in Table 3.

TABLE 3
Summary of ELISA EC₅₀ Values for Seven Selected IgGs

| | Protein ELISA | | | | Peptide ELISA | | | |
|---|---|---|---|---|---|---|---|---|
| | hOPN | | mOPN | | hOPN | | mOPN | |
| Fab | EC₅₀ | Std | EC₅₀ | Std | EC₅₀ | Std | EC₅₀ | Std |
| 6454 | 1.7 | 0.4 | 248.7 | 45.4 | no binding | | no binding | |
| 6455 | 1.6 | 0.2 | 31.4 | 1.4 | no binding | | no binding | |
| 6990 | 2.7 | 0.5 | 1.6 | 0.5 | 1.1 | 0.5 | 1.1 | 0.3 |
| 6991 | 2.7 | 0.6 | 8.5 | 2.3 | 0.6 | 0.4 | 1.9 | 1.3 |
| 6993 | 7.1 | 6.9 | 2.0 | 1.5 | 1.8 | 1.5 | 0.9 | 0.8 |

| | | | | | | |
|---|---|---|---|---|---|---|
| 7202 | 3.7 | 1.0 | 2.5 | 0.8 | no binding | no binding |
| 7212 | 3.7 | 0.4 | 5.3 | 0.1 | no binding | no binding |

Std = standard deviation

### Example 3

### Characterization of HuCAL GOLD® Fabs and IgGs

Selected HuCAL GOLD® Fabs and IgGs were further characterized using several assays as described below, as well as with the ELISA techniques as described in Example 2.

### Cell Adhesion Assay

HuCAL GOLD® Fabs and IgGs were tested for their ability to inhibit OPN mediated adhesion of metastatic breast cancer cell line MDA-MB 435 (ATCC #HTB-129) in a Mn²⁺-dependent, as well as Mn²⁺-independent setup. A Maxisorp™ plate (Nunc Cat. No. #43711) was coated overnight at 4° C. with 50 μL/well of 1 μg/mL hOPN diluted in PBS+ (PBS supplemented with 100 μg/mL CaCl₂, 100 μg/mL MgCl₂) with or without 0.5 mM MnCl₂. The following day, plates were washed twice with PBS+ and blocked with TBS containing 10% BSA for 2 hours at 37° C., 5% CO₂. After blocking, the plates were washed twice with PBS+ and once with adhesion buffer II (HBSS, Gibco #14025-100; 50 nM HEPES Buffer Solution, Gibco #15630-056; 1 mg/mL BSA; 1 mM MnCl₂). HuCAL GOLD® Fabs or IgGs were diluted in adhesion buffer II to the indicated concentrations and 50 μL/well were added. Plates were incubated for 1 hour at 37° C., 5% CO₂.

MDA-MB453 cells were detached using Accutase (PAA Laboratories #L11-007). 1×10⁶ cells/mL were resuspended in adhesion buffer I (HBSS, Gibco #14025-100; 50 nM HEPES Buffer Solution, Gibco #15630-056; 1 mg/mL BSA) and incubated with calcein AM (1 μg/mL/10⁶ cells, Invitrogen #C3099) for 45 minutes at 37° C., 5% CO₂. Cells were centrifuged and resuspended in adhesion buffer II at a concentration of 1×10⁶ cells/mL. 50 μL of cells (1×10⁵ cells/well) were added to antibody containing wells and incubated for 90 minutes at 37° C., 5% CO₂. Adhesion was stopped by gently washing the wells 5 times with adhesion buffer II followed by washing two times with PBS+ leaving 100 μL PBS/well after the last washing step. Fluorescence emission at 535 nm was recorded with excitation at 485 nm.

Cell adhesion data (determined as described above) for twelve selected Fabs are shown below in Table 4. For two of the Fabs (7201 and 7202), no adhesion activity was observed. Fab 7212 was not evaluated. For the Mn2+-independent adhesion assay, IC50 values could not be determined. An inhibitory effect, however, was observed (indicated by a (+)) for all Fabs except 7201 and 7202.

TABLE 4
Summary of Cell Adhesion Data for Twelve Selected Fabs

| | |
|---|---|
| Adhesion Assay | |
| Mn²⁺-dependent | Adhesion Assay |
| hOPN | Mn²⁺-independent |

| Fab | IC₅₀ (nM) | Std | hOPN |
|---|---|---|---|
| 6453 | 117.3 | 13.7 | (+) |
| 6454 | 95.1 | 42.1 | (+) |
| 6455 | 131.4 | 45.5 | (+) |
| 6989 | 66.9 | 27.4 | (+) |
| 6990 | 156.8 | 47.3 | (+) |
| 6991 | 264.3 | 118.7 | (+) |
| 6992 | 98.5 | 40.1 | (+) |
| 6993 | 29.8 | 22.6 | (+) |

| | | |
|---|---|---|
| 7201 | No inhibition | No inhibition |
| 7202 | No inhibition | No inhibition |

| | | | |
|---|---|---|---|
| 7203 | 56.1 | 15.6 | (+) |

| | | |
|---|---|---|
| 7212 | Not evaluated | (+) |

Std = standard deviation

Cell adhesion data (determined as described above) for seven selected IgGs (seven selected Fabs were converted to full length human IgG2 antibodies as described in Example 4) are shown below in Table 5. For two of the IgGs (6455 and 7202), no adhesion activity was observed. For the Mn²⁺-independent adhesion assay, EC₅₀ values could not be determined. An inhibitory effect, however, was observed (indicated by a (+)) for all Fabs except 6455 and 7202.

TABLE 5
Summary of Cell Adhesion Data for Seven Selected IgGs

| | |
|---|---|
| Adhesion Assay | |
| Mn²⁺-dependent | Adhesion Assay |
| hOPN | Mn²⁺-independent |

| IgGs | IC₅₀ (nM) | Std | hOPN |
|---|---|---|---|
| 6454 | 1.3 | 1.4 | (+) |

| | | |
|---|---|---|
| 6455 | No inhibition | No inhibition |

| | | | |
|---|---|---|---|
| 6990 | 0.4 | 0.4 | (+) |
| 6991 | 0.6 | 0.1 | (+) |
| 6993 | 0.4 | 0.4 | (+) |

| | | |
|---|---|---|
| 7202 | No inhibition | No inhibition |

| | | | |
|---|---|---|---|
| 7212 | 9.7 | 4.0 | (+) |

Std = standard deviation

### Affinity Determination Using Solution Equilibrium Titration (SET)

For K_{D} determination by solution equilibrium titration (SET), monomer fractions of antibody protein were used (at least 90% monomer content, analyzed by analytical SEC; Superdex75 (Amersham Pharmacia) for Fab, or Tosoh G3000SWXL (Tosoh Bioscience) for IgG, respectively). Affinity determination in solution was basically performed as described in the literature (Friguet et al., J. Immunol. Methods 77:305 (1985)). In order to improve the sensitivity and accuracy of the SET method, it was transferred from classical ELISA to ECL based technology (Haenel et al., Anal. Biochem. 339:182-184 (2005)). 1 mg/mL goat-anti-human (Fab)₂ fragment specific antibodies (Dianova) were labeled with MSD Sulfo-TAG™ NHS-Ester (Meso Scale Discovery, Gaithersburg, Md., USA) according to the manufacturer's instructions. The experiments were carried out in polypropylene microtiter plates and PBS, pH 7.4, with 0.5% BSA and 0.02% Tween 20 as assay buffer. Unlabeled osteopontin was diluted in a 2n series, starting with a concentration at least 10 times higher than the expected K_{D}. Wells without antigen were used to determine Bₘₐₓ values; wells with assay buffer were used to determine background. After addition of e.g. 25 pM Fab (final concentration in 60 μL final volume), the mixture was incubated over night at room temperature. The applied Fab concentration was similar to or below the expected K_{D}.

Streptavidin coated MSD plates were blocked over night with 3% BSA in PBS (50 μL/well), subsequently the blocking solution was discarded and the plates were coated with 0.2 pg/mL biotinylated osteopontin in assay buffer (30 μL/well) for 1 hour. After washing the coated MSD plates with assay buffer, the equilibrated samples were transferred to those plates (30 μL/well) and incubated for 20 minutes. After washing, 30 μL/well of the MSD Sulfo-tag labeled detection antibody (goat anti-human (Fab)₂) in a final dilution of 1:1000 was added to the MSD plate and incubated for 30 minutes on an Eppendorf shaker (700 rpm).

After washing the plate and adding 30 μL/well MSD Read Buffer T with surfactant, electro-chemiluminescence signals were detected using a Sector Imager 6000 (Meso Scale Discovery, Gaithersburg, Md., USA).

The data was evaluated with XLfit (IDBS) software applying customized fitting models. For K_{D} determination of Fab molecules, a fit model was used according to Haenel et al., Anal. Biochem. 339:182-184 (2005).

A similar protocol was applied to determine K_{D} values for IgG molecules, with the following differences: instead of Fab molecules, whole IgG molecules were added to the dilution series of antigen, and equilibrated over night at room temperature. Subsequently, the samples were treated as described above. KD values for IgG molecules were then determined using a fitting model that was modified according to Piehler et al., J. Immunol. Methods 201:189-192 (1997).

### Biacore K_{D} Determination on Directly Coated Antigen

For K_{D} determination, monomeric Fab fractions (at least 90% monomer content, analyzed by analytical SEC; Superdex75, Amersham Pharmacia) were used as analyte. Binding to immobilized antigen was analyzed using the BIAcore3000 instrument (Biacore, Sweden).

For antigen immobilization, two alternative strategies were used: in the case of human OPN, biotinylated human OPN was bound to a Streptavidin coated sensor chip (Biacore, Sweden) to a binding level of approximately 300 RU. The reference flow cell was coated with a similar amount of biotinylated HSA. In the case of murine OPN, the antigen was immobilized covalently using standard EDC-NHS amine coupling chemistry. CM5 chips (Biacore, Sweden) were coated with murine OPN in 10 mM acetate buffer, pH 3.5 to a level of 400 RU. For the reference flow cell, a respective amount of HSA was used. Regeneration was accomplished with two injections of 10 mM Gly/HCl, pH 1.5 (5 μL) and 50 mM phosphoric acid (5 μL), respectively. Kinetic measurements were done in Dulbeccos PBS, pH 7.4 (Gibco) with 0.05% Tween 20 at a flow rate of 20 μL/min using a 2n serial dilution row of Fab samples. The Fab concentrations ranged from 15.6 to 500 nM. The injection time for each concentration was 1 minute, and the dissociation time was set to a minimum of 3 minutes. A blank injection of running buffer was used for double referencing. All sensorgrams were fitted globally using BIA evaluation software 3.2 (Biacore, Sweden), to determine association and dissociation rate constants (kₒₙ and k_{off}), which were used to calculate the affinity (K_{D}=k_{off}/kₒₙ).

Biacore affinity and/or SET affinity K_{D} data (determined as described above) for twelve selected Fabs are shown below in Table 6.

TABLE 6
Summary of Affinities for Twelve Selected Fabs

| | |
|---|---|
| Biacore Affinity KD (nM) | SET Affinity (nM) |

| Fab | hOPN | mOPN | hOPN |
|---|---|---|---|
| 6453 | 373 | 9 | n.d. |
| 6454 | 16 | 10 | 0.4 |
| 6455 | 29 | 6 | 11 |
| 6989 | 120 | 2065 | n.d. |
| 6990 | 140 | 2272 | 35 |
| 6991 | 300 | 1388 | 3 |
| 6992 | 100 | 2600 | n.d. |
| 6993 | 70 | 3985 | 0.2 |
| 7201 | 20 | 3300 | n.d. |
| 7202 | 220 | 9900 | 6 |
| 7203 | 210 | 1628 | 3 |
| 7212 | n.d. | 2463 | 13 |

n.d. = not determined

### Example 4

### Conversion of Fabs to IgG

In order to express full length IgG, variable domain fragments of heavy (VH) and light chains (VL) were subcloned from Fab expression vectors into appropriate pMorph®_hlg vectors for human IgG2a. Variable domains were amplified via PCR using appropriate oligonucleotides (Fab_HC_for: 5′-CCT ACC GTT CGT CTT CAC CCC TG-3′ (SEQ ID NO:70); Fab_LC_for: 5′-GGC ACT GGC TGG TTT CGC TAC-3′ (SEQ ID NO:71); Fab_HC_rev: 5′-CTC GGA GCC AGC GGA AAC AC-3′ (SEQ ID NO:72); Fab_kappa_rev: 5′-CGG AAA AAT AAA CAC GCT CGG A-3′ (SEQ ID NO:73); Fab_lambda_rev: 5′-GCT CAC ACT CGG TGC GGC TTT C-3′ (SEQ ID NO:74)) followed by digestion with MfeI, BlpI (VH), EcoRV, HpaI (VLlambda) or EcoRV, BsiWI (VKappa), respectively. Fragments were used for subcloning into pMorph®_hlg2κ_{—}1, pMorph®_hlg2λ_{—}1 or pMorph®_hlgG2.

### Transient Expression and Purification of Human IgG2

Transient expression of full length human IgG2 was performed in HKB11 cells, which were transfected with IgG2 heavy and light chain expression vectors. Cell culture supernatant was harvested either three days after transfection or seven days after transfection and upscaling to 3-fold transfection volume, respectively. Supernatant was cleared by centrifugation.

After filtration (0.22 μm or 0.45 μm), the supernatant was subjected to standard protein A affinity chromatography (MabSelect SURE, GE). Proteins were eluted at pH 3 and neutralized in 3 M TRIS, pH 8. Further downstream processing involved buffer exchange to 1× Dubleccos' PBS (Invitrogen) and sterile filtration (0.2 μm; Millipore or Sartorius). Purity of IgG2 was analyzed under denaturing, reducing and denaturing, non-reducing conditions in SDS-PAGE or by capillary electrophoresis. HP-SEC was performed to analyze IgG2 preparations in their native state.

The procedures described above in Examples 1 to 4 were used to produce several fully human anti-OPN IgG₂ antibodies, including antibodies designated as “MOR-6990” (or 6990), “MOR-6991” (or 6991), and “MOR-6993” (or 6993), which are described herein.

### Example 5

### Structural Characterization of Human Antibodies MOR-6990, MOR-6991, and MOR-6993

The cDNA sequences encoding the heavy and light chain variable regions of the MOR-6990, MOR-6991, and MOR-6993 monoclonal antibodies were obtained from the respective hybridomas using standard PCR techniques and were sequenced using standard DNA sequencing techniques.

The nucleotide and amino acid sequences of the heavy chain variable region of 6990 are shown in FIGS. 1A and 1B and in SEQ ID NOs: 9 and 7, respectively. The nucleotide and amino acid sequences of the light chain variable region of 6990 are shown in FIGS. 1C and 1D and in SEQ ID NOs: 10 and 8, respectively.

The nucleotide and amino acid sequences of the heavy chain variable region of 6991 are shown in FIGS. 1E and 1F and in SEQ ID NOs: 23 and 21, respectively. The nucleotide and amino acid sequences of the light chain variable region of 6990 are shown in FIGS. 1G and 1H and in SEQ ID NOs: 24 and 22, respectively.

The nucleotide and amino acid sequences of the heavy chain variable region of 6993 are shown in FIGS. 1I and 1J and in SEQ ID NOs: 37 and 35, respectively. The nucleotide and amino acid sequences of the light chain variable region of 6990 are shown in FIGS. 1K and 1L and in SEQ ID NOs: 38 and 36, respectively.

As described in Example 4, the 6990, 6991, and 6993 human antibodies are of isotype IgG2. The nucleotide and amino acid sequences of the full-length heavy chain for 6990 are shown in FIGS. 2A and 2B, and in SEQ ID NOs: 13 and 11, respectively. The nucleotide and amino acid sequences of the full-length light chain for 6990 are shown in FIGS. 2C and 2D, and in SEQ ID NOs: 14 and 12, respectively.

The nucleotide and amino acid sequences of the full-length heavy chain for 6991 are shown in FIGS. 2E and 2F, and in SEQ ID NO: 27 and 25, respectively. The nucleotide and amino acid sequences of the full-length light chain for 6991 are shown in FIGS. 2G and 2H, and in SEQ ID NO: 28 and 26, respectively.

The nucleotide and amino acid sequences of the full-length heavy chain for 6993 are shown in FIGS. 2I and 2J, and in SEQ ID NO: 41 and 39, respectively. The nucleotide and amino acid sequences of the full-length light chain for 6993 are shown in FIGS. 2K and 2L, and in SEQ ID NO: 42 and 40, respectively.

Comparison of the 6990 heavy chain immunoglobulin sequence to the known human germline immunoglobulin heavy chain sequences demonstrated that the 6990 heavy chain utilizes a V_{H} segment from human germline V_{H} 3-23, a D segment from the human germline 3-22, and a JH segment from human germline JH 4a. Further analysis of the 6990 V_{H} sequence using the Kabat system of CDR region determination led to the delineation of the heavy chain CDR1, CDR2 and CDR3 regions as shown in FIG. 1B, and in SEQ ID NOs: 1, 2 and 3, respectively.

Comparison of the 6990 light chain immunoglobulin sequence to the known human germline immunoglobulin light chain sequences demonstrated that the 6990 light chain utilizes a V_{L} segment from human germline λ3 and a JL segment from human germline JL 3b. Further analysis of the 6990 V_{L} sequence using the Kabat system of CDR region determination led to the delineation of the light chain CDR1, CDR2 and CDR3 regions as shown in FIG. 1D, and in SEQ ID NOs: 4, 5 and 6, respectively.

Comparison of the 6991 heavy chain immunoglobulin sequence to the known human germline immunoglobulin heavy chain sequences demonstrated that the 6991 heavy chain utilizes a V_{H} segment from human germline V_{H} 3-23, a D segment from the human germline 2-21, and a JH segment from human germline JH 4a. Further analysis of the 6991 V_{H} sequence using the Kabat system of CDR region determination led to the delineation of the heavy chain CDR1, CDR2 and CDR3 regions as shown in FIG. 1F, and in SEQ ID NOs: 15, 16 and 17, respectively.

Comparison of the 6991 light chain immunoglobulin sequence to the known human germline immunoglobulin light chain sequences demonstrated that the 6991 light chain utilizes a V_{L} segment from human germline λ1-13 and a JL segment from human germline JL 3b. Further analysis of the 6991 V_{L} sequence using the Kabat system of CDR region determination led to the delineation of the light chain CDR1, CDR2 and CDR3 regions as shown in FIG. 1H, and in SEQ ID NOs: 18, 19 and 20, respectively.

Comparison of the 6993 heavy chain immunoglobulin sequence to the known human germline immunoglobulin heavy chain sequences demonstrated that the 6993 heavy chain utilizes a V_{H} segment from human germline V_{H} 3-23, a D segment from the human germline 3-10, and a JH segment from human germline JH 4a. Further analysis of the 6993 V_{H} sequence using the Kabat system of CDR region determination led to the delineation of the heavy chain CDR1, CDR2 and CDR3 regions as shown in FIG. 1J, and in SEQ ID NOs: 29, 30 and 31, respectively.

Comparison of the 6993 light chain immunoglobulin sequence to the known human germline immunoglobulin light chain sequences demonstrated that the 6993 light chain utilizes a V_{L} segment from human germline λ3 and a JL segment from human germline JL 3b. Further analysis of the 6993 V_{L} sequence using the Kabat system of CDR region determination led to the delineation of the light chain CDR1, CDR2 and CDR3 regions as shown in FIG. 1 L, and in SEQ ID NOs: 32, 33 and 34, respectively.

### Example 6

### Germlined Versions of Human Antibodies MOR-6990, MOR-6991, and MOR-6993

In order to minimize immunogenicity of the 6990, 6991, and 6993 antibodies, several mutations were returned to germ line sequence, as follows. A germlined version of 6990 (6990-GL) was prepared by returning two amino acids in the FR1 region of the heavy variable chain to germ line sequence. The two amino acid residues (and corresponding nucleic acid codons) that were changed in the heavy chain variable region can be seen in FIGS. 1M and 1N, where the mutated residues are indicated by boxing. In the light chain variable region of 6990, five amino acids in the FR1 region and one in the FR3 region were returned to germ line sequence. The six amino amino acids (and corresponding nucleic acid codons) that were changed in the light chain variable region can be seen in FIGS. 1O and 1P, where the mutated residues are indicated by boxing.

A germlined version of 6991 (6991-GL) was prepared by returning two amino acids in the FR1 region of the heavy variable chain to germ line sequence. The two amino acid residues (and corresponding nucleic acid codons) that were changed in the heavy chain variable region can be seen in FIGS. 1Q and 1R, where the mutated residues are indicated by boxing. In the light chain variable region of 6991, two amino acids in the FR1 region and one in the FR3 region were returned to germ line sequence. The three amino amino acids (and corresponding nucleic acid codons) that were changed in the light chain variable region can be seen in FIGS. 1S and 1T, where the mutated residues are indicated by boxing.

A germlined version of 6993 (6993-GL) was prepared by returning two amino acids in the FR1 region of the heavy variable chain to germ line sequence. The two amino acid residues (and corresponding nucleic acid codons) that were changed in the heavy chain variable region can be seen in FIGS. 1U and 1V, where the mutated residues are indicated by boxing. In the light chain variable region of 6993, five amino acids in the FR1 region, one amino acid in the FR2 region, and one in the FR3 region were returned to germ line sequence. The seven amino amino acids (and corresponding nucleic acid codons) that were changed in the light chain variable region can be seen in FIGS. 1W and 1X, where the mutated residues are indicated by boxing.

### Example 7

### Preparation of Mutant to Improve Solubility

In order to improve the solubility of 6993-GL, a point mutation was introduced in the FR2 region of the light chain variable region (V44K). This point mutation (and the corresponding nucleic acid codon) can be seen in FIGS. 1Y and 1Z, where the point mutation is indicated by bold text.

### Example 8

### Characterization of Binding Avidity of Osteopontin Human Monoclonal Antibodies (MOR6990, MOR6991 and MOR6993)

Binding avidity of monoclonal antibodies to Osteopontin was determined using Biacore analysis (General Electric Healthcare, Biacore 3000). To obtain nominal avidity measurements, human Osteopontin (R&D Systems, 1433-OP-050/CF) and mouse Osteopontin (R&D Systems, 441-OP-050/CF) were immobilized on a biosensor chip using standard amine coupling and various concentrations of monoclonal antibodies (MOR6990, MOR6991 and MOR6993) were flowed across the surface at 25.0° C. in 10 mM HEPES pH 7.4, 150 mM NaCl, 0.005% P20. The binding data were fit globally to a simple one-to-one binding model. The results are shown in Table 7.

TABLE 7
Summary of Avidities for MOR6990, MOR6991, and MOR6993

| | Mouse Osteopontin | | | Human Osteopontin | | |
|---|---|---|---|---|---|---|
| | kₐ (1/Ms) | k_{d} (1/s) | K_{D} (Avidity) | kₐ (1/Ms) | k_{d} (1/s) | K_{D} (Avidity) |
| MOR6990 | 1.83E+05 | 8.64E−04 | 4.7 nM | 9.69E+04 | 2.03E−03 | 20.9 nM |
| MOR6991 | N/A | N/A | N/A | 4.91E+05 | 9.33E−03 | 19.0 nM |
| MOR6993 | 6.64E+06 | 4.96E−03 | 746.7 pM | 6.14E+06 | 4.62E−03 | 752.0 pM |

N/A = No detectable binding of MOR6991 to mouse Osteopontin

### Example 9

### Inhibition of In Vivo Tumor Growth and Metastasis by MOR-6993

The following study demonstrated the anti-metastatic efficacy of MOR-6993 in a preclinical model of breast cancer.

MDA-MB-435-Luc is a human breast cancer cell line transfected with the luciferase gene. When implanted into the mouse mammary fat pad (MFP), it induces formation of tumors that can be measured by bioluminescence imaging (BLI).

In this study, MDA-MB-435-Luc cells were injected (3×10⁶ per animal) in the mammary fat pad of immuno-compromised SCID BALB/c mice that had received a pre-dose of MOR-6993 antibody (30 mg/kg, n=10 per group) 24 hours before implantation. Following implantation, animals were dosed once a week subcutaneously with 6993 (10 mg/kg) for 6 weeks. Bioluminescence of individual animals was measured once a week for 10 weeks. Tumors were surgically removed on day 40 from implant, and animals were monitored for additional 50 days for appearance of metastasis as well as overall survival.

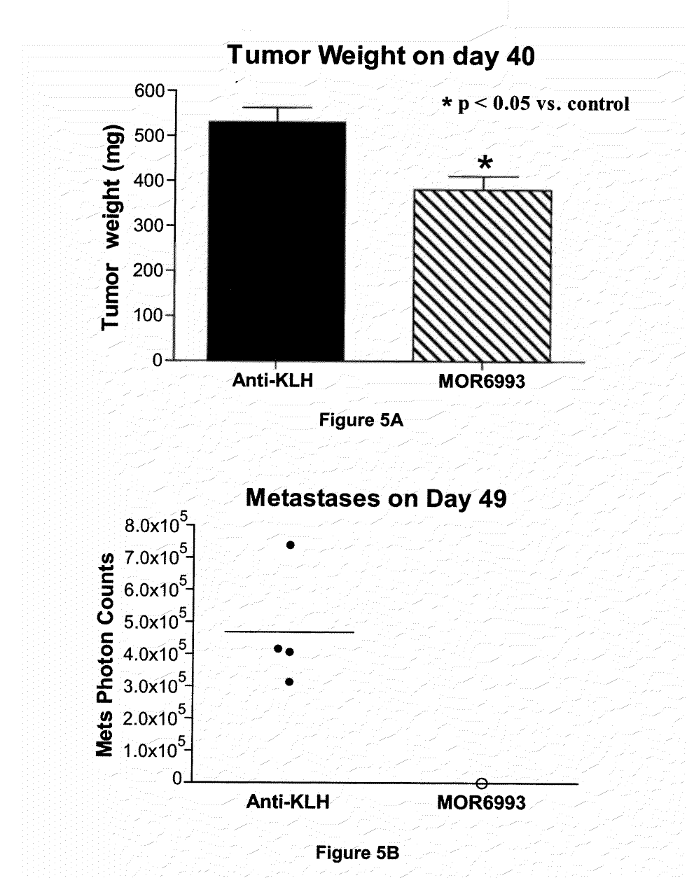

Dosing with 6993 resulted in significant TGI (tumor growth inhibition), 30% tumor weight reduction as shown in FIG. 5A. In addition, the treatment prevented or delayed the appearance of metastases (FIG. 5B) and improved overall survival. RNA analysis of the tumors upon removal showed that treatment with 6993 induced a decrease in OPN mRNA expression.

### Example 10

### In Vivo Neutralization of Circulating Osteopontin

The following study demonstrated neutralization of both endogenous mouse and tumor-produced Osteopontin by Mor-6990 and Mor-6993.

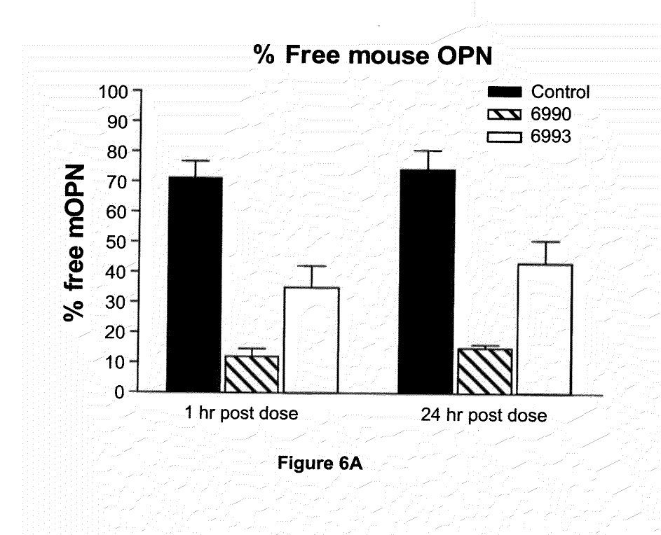

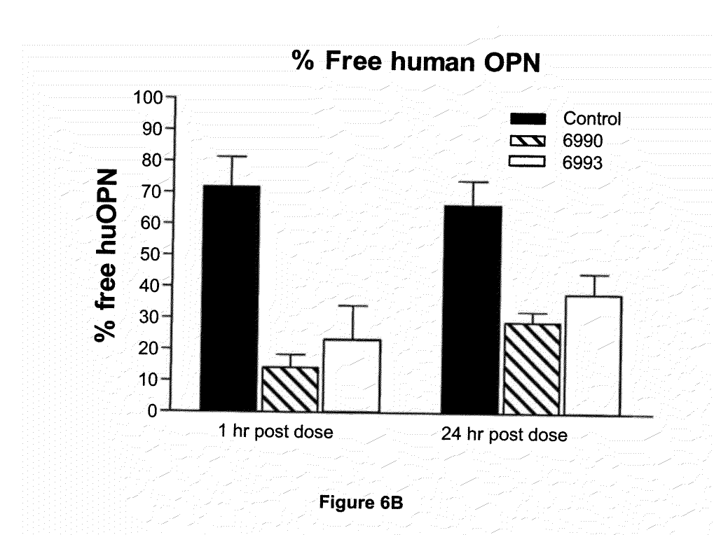

MDA-MB-435-Hal/Luc cells were injected (3×10⁶ per animal) in the mammary fat pad of immuno-compromised SCID BALB/c mice to induce tumor formation. When the tumors reached 500 mm³, animals were randomized and dosed intravenously with Mor-6990 or Mor-6993 at 25 mg/kg. Blood was collected at 1 and 24 hr post-dose. Total mouse and human Osteopontin in plasma were analyzed using a commercial ELISA kit (R&D systems). For analysis of the free Osteopontin, plasma was treated overnight with Protein G magnetic beads to remove Osteopontin bound to the IgGs. Free Osteopontin levels were determined using a commercial ELISA kit (R&D systems). The results demonstrated that both Mor-6990 and Mor-6993 effectively neutralized both mouse (FIG. 6A) and human (FIG. 6B) plasma Osteopontin.

### Example 11

### ELISA EC₅₀ Values and Binding Affinities of MOR-10475 Fabs

Protein ELISA EC₅₀ values, peptide ELISA EC₅₀ values, and Biacore K_{D} values were determined for MOR-10475 Fabs using both human and mouse osteopontin in a similar manner to procedures described in Examples 2 and 3, and the results are shown in Table 8.

TABLE 8
ELISA EC₅₀ and Biacore K_{D} values of MOR-10475

| | Protein ELISA | | Peptide ELISA | | Biacore | |
|---|---|---|---|---|---|---|
| | hOPN | mOPN | hOPN | mOPN | hOPN | mOPN |
| Fab | EC₅₀ (nM) | EC₅₀ (nM) | EC₅₀ (nM) | EC₅₀ (nM) | K_{D} (nM) | K_{D} (nM) |
| 10475 | 0.6 | 0.6 | 0.7 | 0.7 | 80 | 41 |

SUMMARY OF SEQUENCE LISTING (H-CDR1=heavy chain CDR1; L-CDR1=light chain CDR1, etc; 6990=antibody MOR-6990 as described herein; 6991=antibody MOR-6991 as described herein; 6993=antibody MOR-6993 as described herein; VH=variable heavy region; VL=variable light region; Heavy=full-length heavy chain; Light=full-length light chain; 6990-GL=germlined version of MOR-6990, as described herein; 6991-GL=germlined version of MOR-6991, as described herein; 6993-GL=germlined version of MOR-6993, as described herein; 6993-GL-V44K=germlined version of MOR-6993, including the V44K point mutation, as described herein; a.a.=amino acid; n.a.=nucleic acid)

| SEQ ID NO: | Description |
|---|---|
| 1 | H-CDR1 a.a. 6990 |
| 2 | H-CDR2 a.a. 6990 |
| 3 | H-CDR3 a.a. 6990 |
| 4 | L-CDR1 a.a. 6990 |
| 5 | L-CDR2 a.a. 6990 |
| 6 | L-CDR3 a.a. 6990 |
| 7 | V_{H} a.a. 6990 |
| 8 | V_{L} a.a. 6990 |
| 9 | V_{H} n.a. 6990 |
| 10 | V_{L} n.a. 6990 |
| 11 | Heavy a.a. 6990 |
| 12 | Light a.a. 6990 |
| 13 | Heavy n.a. 6990 |
| 14 | Light n.a. 6990 |
| 15 | H-CDR1 a.a. 6991 |
| 16 | H-CDR2 a.a. 6991 |
| 17 | H-CDR3 a.a. 6991 |
| 18 | L-CDR1 a.a. 6991 |
| 19 | L-CDR2 a.a. 6991 |
| 20 | L-CDR3 a.a. 6991 |
| 21 | V_{H} a.a. 6991 |
| 22 | V_{L} a.a. 6991 |
| 23 | V_{H} n.a. 6991 |
| 24 | V_{L} n.a. 6991 |
| 25 | Heavy a.a. 6991 |
| 26 | Light a.a. 6991 |
| 27 | Heavy n.a. 6991 |
| 28 | Light n.a. 6991 |
| 29 | H-CDR1 a.a. 6993 |
| 30 | H-CDR2 a.a. 6993 |
| 31 | H-CDR3 a.a. 6993 |
| 32 | L-CDR1 a.a. 6993 |
| 33 | L-CDR2 a.a. 6993 |
| 34 | L-CDR3 a.a. 6993 |
| 35 | V_{H} a.a. 6993 |
| 36 | V_{L} a.a. 6993 |
| 37 | V_{H} n.a. 6993 |
| 38 | V_{L} n.a. 6993 |
| 39 | Heavy a.a. 6993 |
| 40 | Light a.a. 6993 |
| 41 | Heavy n.a. 6993 |
| 42 | Light n.a. 6993 |
| 43 | Full-length human |
| | OPN a.a. isoform b |
| 44 | V_{H} a.a. 6990-GL |
| 45 | V_{H} n.a. 6990-GL |
| 46 | V_{L} a.a. 6990-GL |
| 47 | V_{L} n.a. 6990-GL |
| 48 | V_{H} a.a. 6991-GL |
| 49 | V_{H} n.a. 6991-GL |
| 50 | V_{L} a.a. 6991-GL |
| 51 | V_{L} n.a. 6991-GL |
| 52 | V_{H} a.a. 6993-GL |
| 53 | V_{H} n.a. 6993-GL |
| 54 | V_{L} a.a. 6993-GL |
| 55 | V_{L} n.a. 6993-GL |
| 56 | V_{L} a.a. 6993-GL-V44K |
| 57 | V_{L} n.a. 6993-GL-V44K |
| 58 | Heavy 6990-GL a.a. |
| 59 | Light 6990-GL a.a. |
| 60 | Heavy 6990-GL n.a. |
| 61 | Light 6990-GL n.a. |
| 62 | Heavy 6991-GL a.a. |
| 63 | Light 6991-GL a.a. |
| 64 | Heavy 6991-GL n.a. |
| 65 | Light 6991-GL n.a. |
| 66 | Heavy 6993-GL a.a. |
| 67 | Light 6993-GL a.a. |
| 68 | Heavy 6993-GL n.a. |
| 69 | Light 6993-GL n.a. |
| 70 | Fab heavy chain |
| | forward primer |
| 71 | Fab light chain forward |
| | primer |
| 72 | Fab heavy chain |
| | reverse primer |
| 73 | Fab kappa reverse |
| | primer |
| 74 | Fab lambda reverse |
| | primer |
| 75 | L-CDR3 a.a. |
| | MOR10475 |
| 76 | V_{L} a.a. MOR10475 |
| 77 | V_{L} n.a. MOR10475 |
| 78 | Light a.a. MOR10475 |

We claim:

1. An isolated antibody, or antigen-binding portion thereof, that specifically binds to osteopontin, wherein said antibody or antigen-binding portion comprises:
(a) an H-CDR1 as set forth in SEQ ID NO:1, an H-CDR2 as set forth in SEQ ID NO:2, an H-CDR3 as set forth in SEQ ID NO:3, an L-CDR1 as set forth in SEQ ID NO:4, an L-CDR2 as set forth in SEQ ID NO:5, and an L-CDR3 as set forth in SEQ ID NO:6;
(b) an H-CDR1 as set forth in SEQ ID NO:15, an H-CDR2 as set forth in SEQ ID NO:16, an H-CDR3 as set forth in SEQ ID NO:17, an L-CDR1 as set forth in SEQ ID NO:18, an L-CDR2 as set forth in SEQ ID NO:19, and an L-CDR3 as set forth in SEQ ID NO:20;
(c) an H-CDR1 as set forth in SEQ ID NO:29, an H-CDR2 as set forth in SEQ ID NO:30, an H-CDR3 as set forth in SEQ ID NO:31, an L-CDR1 as set forth in SEQ ID NO:32, an L-CDR2 as set forth in SEQ ID NO:33, and an L-CDR3 as set forth in SEQ ID NO:34; or
(d) an H-CDR1 as set forth in SEQ ID NO:1, an H-CDR2 as set forth in SEQ ID NO:2, an H-CDR3 as set forth in SEQ ID NO:3, an L-CDR1 as set forth in SEQ ID NO:4, an L-CDR2 as set forth in SEQ ID NO:5, and an L-CDR3 as set forth in SEQ ID NO:75.

2. The antibody according to claim 1, which is an IgG1 or IgG2.

3. The antibody according to claim 1, which is a human, humanized, or chimeric antibody.

4. The antibody according to claim 3, which is a synthetic human antibody.

5. The antigen-binding portion according to claim 1, which is a Fab or scFv antibody fragment.

6. A nucleic acid encoding the antibody or antigen-binding portion according to claim 1.

7. A vector comprising the nucleic acid according to claim 6.

8. A host cell comprising the vector according to claim 7.

9. The host cell according to claim 8, wherein said cell is bacterial.

10. The host cell according to claim 8, wherein said cell is mammalian.

11. A pharmaceutical composition comprising an antibody or antigen-binding portion according to claim 1 and a pharmaceutically acceptable carrier or excipient.

12. A method for treating abnormal cell growth comprising administering to a subject in need thereof an effective amount of a pharmaceutical composition according to claim 11.

13. A method of reducing tumor cell metastasis in a subject, comprising administering to said subject an effective amount of a pharmaceutical composition according to claim 11.

14. A method of preparing an anti-ostepontin antibody, or antigen-binding portion thereof, comprising expressing the antibody or antigen-binding portion in a host cell according to claim 8.

15. An isolated antibody, or antigen-binding portion thereof, that specifically binds to osteopontin, wherein said antibody or antigen-binding portion comprises a V_{H} chain amino acid sequence as set forth in SEQ ID NO:7, SEQ ID NO:21, SEQ ID NO:35, SEQ ID NO:44, SEQ ID NO:48, or SEQ ID NO:52.

16. The antibody or antigen-binding portion according to claim 15, wherein said antibody or antigen-binding portion further comprises a V_{L} chain amino acid sequence as set forth in SEQ ID NO:8, SEQ ID NO:22, SEQ ID NO:36, SEQ ID NO:46, SEQ ID NO:50, SEQ ID NO:54, or SEQ ID NO:76.

17. An isolated antibody that specifically binds to osteopontin, wherein said antibody comprises a heavy chain amino acid sequence as set forth in SEQ ID NO:11, SEQ ID NO:25, SEQ ID NO:39, SEQ ID NO:58, SEQ ID NO:62, or SEQ ID NO:66, with the proviso that the C-terminal lysine residue of said heavy chain amino acid sequence is optionally not present.

18. The isolated antibody according to claim 17, further comprising a light chain amino acid sequence as set forth in SEQ ID NO:12, SEQ ID NO:26, SEQ ID NO:40, SEQ ID NO:59, SEQ ID NO:63, SEQ ID NO:67, or SEQ ID NO:78.

19. An isolated antibody or antigen-binding portion thereof comprising a V_{H} chain that is encoded by (i) a nucleic acid sequence comprising SEQ ID NO:9, SEQ ID NO:23, SEQ ID NO:37, SEQ ID NO:45, SEQ ID NO:49, or SEQ ID NO:53, or (ii) a nucleic acid sequence that hybridizes under high stringency conditions to the complementary strand of SEQ ID NO:9, SEQ ID NO:23, SEQ ID NO:37, SEQ ID NO:45, SEQ ID NO:49, or SEQ ID NO:53, wherein said antibody or antigen-binding portion specifically binds to osteopontin.

20. The isolated antibody or antigen-binding portion according to claim 19, further comprising a V_{L} chain that is encoded by (i) a nucleic acid sequence comprising SEQ ID NO:10, SEQ ID NO:24, SEQ ID NO:38, SEQ ID NO:47, SEQ ID NO:51, SEQ ID NO:55, or SEQ ID NO:77 or (ii) a nucleic acid sequence that hybridizes under high stringency conditions to the complementary strand of SEQ ID NO:10, SEQ ID NO:24, SEQ ID NO:38, SEQ ID NO:47, SEQ ID NO:51, SEQ ID NO:55, or SEQ ID NO:77, wherein said antibody or antigen-binding portion specifically binds to osteopontin.


SEQUENCE LISTING (78 sequences)

SEQ ID NO: 1 - protein, 6 aa, Homo sapiens
SNYVMH

SEQ ID NO: 2 - protein, 17 aa, Homo sapiens
SIFGSGSDTYYADSVKG

SEQ ID NO: 3 - protein, 16 aa, Homo sapiens
RSASSGFGFAGYGIDS

SEQ ID NO: 4 - protein, 11 aa, Homo sapiens
SGDSLRYYYAH

SEQ ID NO: 5 - protein, 7 aa, Homo sapiens
DDNKRPS

SEQ ID NO: 6 - protein, 10 aa, Homo sapiens
QSWDLFHSSV

SEQ ID NO: 7 - protein, 124 aa, Homo sapiens
QVQLVESGGGLVQPGGSLRLSCAASGFTFSNYVMHWVRQAPGKGLEWVSSIFGSGSDTYYADSVKGRFTISRDNSKNTLYLQMNSLRAEDTAVYYCARSASSGFGFAGYGIDSWGQGTLVTVSS

SEQ ID NO: 8 - protein, 107 aa, Homo sapiens
DIELTQPPSVSVAPGQTARISCSGDSLRYYYAHWYQQKPGQAPVLVIYDDNKRPSGIPERFSGSNSGNTATLTISGTQAEDEADYYCQSWDLFHSSVFGGGTKLTVL

SEQ ID NO: 9 - DNA, 372 bp, Homo sapiens
caggtgcaattggtggaaagcggcggcggcctggtgcaaccgggcggcagcctgcgtctgagctgcgcggcctccggatttaccttttctaattatgttatgcattgggtgcgccaagcccctgggaagggtctcgagtgggtgagctctatctttggttctggtagcgatacctattatgcggatagcgtgaaaggccgttttaccatttcacgtgataattcgaaaaacaccctgtatctgcaaatgaacagcctgcgtgcggaagatacggccgtgtattattgcgcgcggtctgcttcttctggttttggttttgctggttatggtattgattcttggggccaaggcaccctggtgacggttagctca

SEQ ID NO: 10 - DNA, 321 bp, Homo sapiens
gatatcgaactgacccagccgccttcagtgagcgttgcaccaggtcagaccgcgcgtatctcgtgtagcggcgattctcttcgttattattatgctcattggtaccagcagaaacccgggcaggcgccagttcttgtgatttatgatgataataagcgtccctcaggcatcccggaacgctttagcggatccaacagcggcaacaccgcgaccctgaccattagcggcactcaggcggaagacgaagcggattattattgccagtcttgggatctttttcattcttctgtgtttggcggcggcacgaagttaaccgtccta

SEQ ID NO: 11 - protein, 450 aa, Homo sapiens
QVQLVESGGGLVQPGGSLRLSCAASGFTFSNYVMHWVRQAPGKGLEWVSSIFGSGSDTYYADSVKGRFTISRDNSKNTLYLQMNSLRAEDTAVYYCARSASSGFGFAGYGIDSWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 213 aa, Homo sapiens
DIELTQPPSVSVAPGQTARISCSGDSLRYYYAHWYQQKPGQAPVLVIYDDNKRPSGIPERFSGSNSGNTATLTISGTQAEDEADYYCQSWDLFHSSVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - DNA, 1350 bp, Homo sapiens
caggtgcaattggtggaaagcggcggcggcctggtgcaaccgggcggcagcctgcgtctgagctgcgcggcctccggatttaccttttctaattatgttatgcattgggtgcgccaagcccctgggaagggtctcgagtgggtgagctctatctttggttctggtagcgatacctattatgcggatagcgtgaaaggccgttttaccatttcacgtgataattcgaaaaacaccctgtatctgcaaatgaacagcctgcgtgcggaagatacggccgtgtattattgcgcgcggtctgcttcttctggttttggttttgctggttatggtattgattcttggggccaaggcaccctggtgacggttagctcagccagcaccaagggccccagcgtgttccccctggccccctgcagcagaagcaccagcgagagcacagccgccctgggctgcctggtgaaggactacttccccgagcccgtgaccgtgagctggaacagcggagccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagcggaagtgctgcgtggagtgccccccctgccctgcccctcctgtggccggaccctccgtgttcctgttcccccccaagcccaaggacaccctgatgatcagccggacccccgaggtgacctgcgtggtggtggacgtgagccacgaggaccccgaggtgcagtttaattggtacgtggacggcgtggaggtgcacaacgccaagaccaagccccgggaggaacagttcaacagcaccttccgggtggtgtccgtgctgaccgtggtgcaccaggactggctgaacggcaaagaatacaagtgcaaggtgtccaacaagggcctgcctgcccccatcgagaaaaccatcagcaagacaaagggccagcccagggaaccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagccggtggcagcagggcaacgtgttcagctgcagcgtgatgcacgaggccctgcacaaccactacacccagaagagcctgagcctgtcccccggcaaa

SEQ ID NO: 14 - DNA, 639 bp, Homo sapiens
gatatcgaactgacccagccgccttcagtgagcgttgcaccaggtcagaccgcgcgtatctcgtgtagcggcgattctcttcgttattattatgctcattggtaccagcagaaacccgggcaggcgccagttcttgtgatttatgatgataataagcgtccctcaggcatcccggaacgctttagcggatccaacagcggcaacaccgcgaccctgaccattagcggcactcaggcggaagacgaagcggattattattgccagtcttgggatctttttcattcttctgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 15 - protein, 6 aa, Homo sapiens
NNYAVS

SEQ ID NO: 16 - protein, 17 aa, Homo sapiens
GISYGGSNTYYADSVKG

SEQ ID NO: 17 - protein, 9 aa, Homo sapiens
RTLGGDFDH

SEQ ID NO: 18 - protein, 13 aa, Homo sapiens
SGSSSNIGSNYVN

SEQ ID NO: 19 - protein, 7 aa, Homo sapiens
GNSKRPS

SEQ ID NO: 20 - protein, 9 aa, Homo sapiens
QSFTQMLLV

SEQ ID NO: 21 - protein, 117 aa, Homo sapiens
QVQLVESGGGLVQPGGSLRLSCAASGFTFNNYAVSWVRQAPGKGLEWVSGISYGGSNTYYADSVKGRFTISRDNSKNTLYLQMNSLRAEDTAVYYCARTLGGDFDHWGQGTLVTVSS

SEQ ID NO: 22 - protein, 108 aa, Homo sapiens
DIVLTQPPSVSGAPGQRVTISCSGSSSNIGSNYVNWYQQLPGTAPKLLIYGNSKRPSGVPDRFSGSKSGTSASLAITGLQSEDEADYYCQSFTQMLLVFGGGTKLTVL

SEQ ID NO: 23 - DNA, 351 bp, Homo sapiens
caggtgcaattggtggaaagcggcggcggcctggtgcaaccgggcggcagcctgcgtctgagctgcgcggcctccggatttacctttaataattatgctgtttcttgggtgcgccaagcccctgggaagggtctcgagtgggtgagcggtatctcttatggtggtagcaatacctattatgcggatagcgtgaaaggccgttttaccatttcacgtgataattcgaaaaacaccctgtatctgcaaatgaacagcctgcgtgcggaagatacggccgtgtattattgcgcgcgtactcttggtggtgattttgatcattggggccaaggcaccctggtgacggttagctca

SEQ ID NO: 24 - DNA, 324 bp, Homo sapiens
gatatcgtgctgacccagccgccttcagtgagtggcgcaccaggtcagcgtgtgaccatctcgtgtagcggcagcagcagcaacattggttctaattatgtgaattggtaccagcagttgcccgggacggcgccgaaacttctgatttatggtaattctaagcgtccctcaggcgtgccggatcgttttagcggatccaaaagcggcaccagcgcgagccttgcgattacgggcctgcaaagcgaagacgaagcggattattattgccagtcttttactcagatgcttcttgtgtttggcggcggcacgaagttaaccgtccta

SEQ ID NO: 25 - protein, 443 aa, Homo sapiens
QVQLVESGGGLVQPGGSLRLSCAASGFTFNNYAVSWVRQAPGKGLEWVSGISYGGSNTYYADSVKGRFTISRDNSKNTLYLQMNSLRAEDTAVYYCARTLGGDFDHWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 26 - protein, 214 aa, Homo sapiens
DIVLTQPPSVSGAPGQRVTISCSGSSSNIGSNYVNWYQQLPGTAPKLLIYGNSKRPSGVPDRFSGSKSGTSASLAITGLQSEDEADYYCQSFTQMLLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 27 - DNA, 1329 bp, Homo sapiens
caggtgcaattggtggaaagcggcggcggcctggtgcaaccgggcggcagcctgcgtctgagctgcgcggcctccggatttacctttaataattatgctgtttcttgggtgcgccaagcccctgggaagggtctcgagtgggtgagcggtatctcttatggtggtagcaatacctattatgcggatagcgtgaaaggccgttttaccatttcacgtgataattcgaaaaacaccctgtatctgcaaatgaacagcctgcgtgcggaagatacggccgtgtattattgcgcgcgtactcttggtggtgattttgatcattggggccaaggcaccctggtgacggttagctcagccagcaccaagggccccagcgtgttccccctggccccctgcagcagaagcaccagcgagagcacagccgccctgggctgcctggtgaaggactacttccccgagcccgtgaccgtgagctggaacagcggagccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagcggaagtgctgcgtggagtgccccccctgccctgcccctcctgtggccggaccctccgtgttcctgttcccccccaagcccaaggacaccctgatgatcagccggacccccgaggtgacctgcgtggtggtggacgtgagccacgaggaccccgaggtgcagtttaattggtacgtggacggcgtggaggtgcacaacgccaagaccaagccccgggaggaacagttcaacagcaccttccgggtggtgtccgtgctgaccgtggtgcaccaggactggctgaacggcaaagaatacaagtgcaaggtgtccaacaagggcctgcctgcccccatcgagaaaaccatcagcaagacaaagggccagcccagggaaccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagccggtggcagcagggcaacgtgttcagctgcagcgtgatgcacgaggccctgcacaaccactacacccagaagagcctgagcctgtcccccggcaaa

SEQ ID NO: 28 - DNA, 642 bp, Homo sapiens
gatatcgtgctgacccagccgccttcagtgagtggcgcaccaggtcagcgtgtgaccatctcgtgtagcggcagcagcagcaacattggttctaattatgtgaattggtaccagcagttgcccgggacggcgccgaaacttctgatttatggtaattctaagcgtccctcaggcgtgccggatcgttttagcggatccaaaagcggcaccagcgcgagccttgcgattacgggcctgcaaagcgaagacgaagcggattattattgccagtcttttactcagatgcttcttgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 29 - protein, 6 aa, Homo sapiens
TTSSMH

SEQ ID NO: 30 - protein, 17 aa, Homo sapiens
RISSHGSNTYYADSVKG

SEQ ID NO: 31 - protein, 12 aa, Homo sapiens
RDMYRGVYGFAL

SEQ ID NO: 32 - protein, 11 aa, Homo sapiens
SGDAIRNYYVH

SEQ ID NO: 33 - protein, 7 aa, Homo sapiens
EDSDRPS

SEQ ID NO: 34 - protein, 9 aa, Homo sapiens
QSYDKSNVV

SEQ ID NO: 35 - protein, 120 aa, Homo sapiens
QVQLVESGGGLVQPGGSLRLSCAASGFTFTTSSMHWVRQAPGKGLEWVSRISSHGSNTYYADSVKGRFTISRDNSKNTLYLQMNSLRAEDTAVYYCARDMYRGVYGFALWGQGTLVTVSS

SEQ ID NO: 36 - protein, 106 aa, Homo sapiens
DIELTQPPSVSVAPGQTARISCSGDAIRNYYVHWYQQKPGQAPVLVIYEDSDRPSGIPERFSGSNSGNTATLTISGTQAEDEADYYCQSYDKSNVVFGGGTKLTVL

SEQ ID NO: 37 - DNA, 360 bp, Homo sapiens
caggtgcaattggtggaaagcggcggcggcctggtgcaaccgggcggcagcctgcgtctgagctgcgcggcctccggatttacctttactacttcttctatgcattgggtgcgccaagcccctgggaagggtctcgagtgggtgagccgtatctcttctcatggtagcaatacctattatgcggatagcgtgaaaggccgttttaccatttcacgtgataattcgaaaaacaccctgtatctgcaaatgaacagcctgcgtgcggaagatacggccgtgtattattgcgcgcgtgatatgtatcgtggtgtttatggttttgctctttggggccaaggcaccctggtgacggttagctca

SEQ ID NO: 38 - DNA, 318 bp, Homo sapiens
gatatcgaactgacccagccgccttcagtgagcgttgcaccaggtcagaccgcgcgtatctcgtgtagcggcgatgctattcgtaattattatgttcattggtaccagcagaaacccgggcaggcgccagttcttgtgatttatgaggattctgatcgtccctcaggcatcccggaacgctttagcggatccaacagcggcaacaccgcgaccctgaccattagcggcactcaggcggaagacgaagcggattattattgccagtcttatgataagtctaatgttgtgtttggcggcggcacgaagttaaccgtccta

SEQ ID NO: 39 - protein, 446 aa, Homo sapiens
QVQLVESGGGLVQPGGSLRLSCAASGFTFTTSSMHWVRQAPGKGLEWVSRISSHGSNTYYADSVKGRFTISRDNSKNTLYLQMNSLRAEDTAVYYCARDMYRGVYGFALWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 40 - protein, 212 aa, Homo sapiens
DIELTQPPSVSVAPGQTARISCSGDAIRNYYVHWYQQKPGQAPVLVIYEDSDRPSGIPERFSGSNSGNTATLTISGTQAEDEADYYCQSYDKSNVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - DNA, 1338 bp, Homo sapiens
caggtgcaattggtggaaagcggcggcggcctggtgcaaccgggcggcagcctgcgtctgagctgcgcggcctccggatttacctttactacttcttctatgcattgggtgcgccaagcccctgggaagggtctcgagtgggtgagccgtatctcttctcatggtagcaatacctattatgcggatagcgtgaaaggccgttttaccatttcacgtgataattcgaaaaacaccctgtatctgcaaatgaacagcctgcgtgcggaagatacggccgtgtattattgcgcgcgtgatatgtatcgtggtgtttatggttttgctctttggggccaaggcaccctggtgacggttagctcagccagcaccaagggccccagcgtgttccccctggccccctgcagcagaagcaccagcgagagcacagccgccctgggctgcctggtgaaggactacttccccgagcccgtgaccgtgagctggaacagcggagccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagcggaagtgctgcgtggagtgccccccctgccctgcccctcctgtggccggaccctccgtgttcctgttcccccccaagcccaaggacaccctgatgatcagccggacccccgaggtgacctgcgtggtggtggacgtgagccacgaggaccccgaggtgcagtttaattggtacgtggacggcgtggaggtgcacaacgccaagaccaagccccgggaggaacagttcaacagcaccttccgggtggtgtccgtgctgaccgtggtgcaccaggactggctgaacggcaaagaatacaagtgcaaggtgtccaacaagggcctgcctgcccccatcgagaaaaccatcagcaagacaaagggccagcccagggaaccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagccggtggcagcagggcaacgtgttcagctgcagcgtgatgcacgaggccctgcacaaccactacacccagaagagcctgagcctgtcccccggcaaa

SEQ ID NO: 42 - DNA, 636 bp, Homo sapiens
gatatcgaactgacccagccgccttcagtgagcgttgcaccaggtcagaccgcgcgtatctcgtgtagcggcgatgctattcgtaattattatgttcattggtaccagcagaaacccgggcaggcgccagttcttgtgatttatgaggattctgatcgtccctcaggcatcccggaacgctttagcggatccaacagcggcaacaccgcgaccctgaccattagcggcactcaggcggaagacgaagcggattattattgccagtcttatgataagtctaatgttgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 43 - protein, 300 aa, Homo sapiens
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 44 - protein, 124 aa, Homo sapiens
EVQLLESGGGLVQPGGSLRLSCAASGFTFSNYVMHWVRQAPGKGLEWVSSIFGSGSDTYYADSVKGRFTISRDNSKNTLYLQMNSLRAEDTAVYYCARSASSGFGFAGYGIDSWGQGTLVTVSS

SEQ ID NO: 45 - DNA, 372 bp, Homo sapiens
gaggtgcagctgctggagagcggcggcggcctggtgcagcccggcggcagcctgaggctgtcctgcgccgccagcggcttcaccttcagcaactacgtgatgcactgggtgaggcaggcccccggcaagggcctggagtgggtgagcagcatcttcggcagcggcagcgacacctactacgccgacagcgtgaagggcaggttcaccatcagcagggacaacagcaagaacaccctgtacctgcagatgaacagcctgagggccgaggacaccgccgtgtactactgcgccaggagcgccagcagcggcttcggcttcgccggctacggcatcgacagctggggccagggcaccctggtgaccgtgagctca

SEQ ID NO: 46 - protein, 107 aa, Homo sapiens
SYELTQPPSVSVSPGQTASITCSGDSLRYYYAHWYQQKPGQSPVLVIYDDNKRPSGIPERFSGSNSGNTATLTISGTQAMDEADYYCQSWDLFHSSVFGGGTKLTVL

SEQ ID NO: 47 - DNA, 321 bp, Homo sapiens
agctacgagctgacccagccccccagcgtgagcgtgagccccggccagaccgccagcatcacctgcagcggcgacagcctgaggtactactacgcccactggtaccagcagaagcccggccagagccccgtgctggtgatctacgacgacaacaagaggcccagcggcatccccgagaggttcagcggcagcaacagcggcaacaccgccaccctgaccatcagcggcacccaggccatggacgaggccgactactactgccagagctgggacctgttccacagcagcgtgttcggcggcggcaccaagttaaccgtccta

SEQ ID NO: 48 - protein, 117 aa, Homo sapiens
EVQLLESGGGLVQPGGSLRLSCAASGFTFNNYAVSWVRQAPGKGLEWVSGISYGGSNTYYADSVKGRFTISRDNSKNTLYLQMNSLRAEDTAVYYCARTLGGDFDHWGQGTLVTVSS

SEQ ID NO: 49 - DNA, 351 bp, Homo sapiens
gaggtgcaattgctggaaagcggcggcggcctggtgcaaccgggcggcagcctgcgtctgagctgcgcggcctccggatttacctttaataattatgctgtttcttgggtgcgccaagcccctgggaagggtctcgagtgggtgagcggtatctcttatggtggtagcaatacctattatgcggatagcgtgaaaggccgttttaccatttcacgtgataattcgaaaaacaccctgtatctgcaaatgaacagcctgcgtgcggaagatacggccgtgtattattgcgcgcgtactcttggtggtgattttgatcattggggccaaggcaccctggtgacggttagctca

SEQ ID NO: 50 - protein, 108 aa, Homo sapiens
QSVLTQPPSVSGAPGQRVTISCSGSSSNIGSNYVNWYQQLPGTAPKLLIYGNSKRPSGVPDRFSGSKSGTSASLAITGLQAEDEADYYCQSFTQMLLVFGGGTKLTVL

SEQ ID NO: 51 - DNA, 324 bp, Homo sapiens
cagagcgtgctgacccagccgccttcagtgagtggcgcaccaggtcagcgtgtgaccatctcgtgtagcggcagcagcagcaacattggttctaattatgtgaattggtaccagcagttgcccgggacggcgccgaaacttctgatttatggtaattctaagcgtccctcaggcgtgccggatcgttttagcggatccaaaagcggcaccagcgcgagccttgcgattacgggcctgcaagccgaagacgaagcggattattattgccagtcttttactcagatgcttcttgtgtttggcggcggcacgaagttaaccgtccta

SEQ ID NO: 52 - protein, 120 aa, Homo sapiens
EVQLLESGGGLVQPGGSLRLSCAASGFTFTTSSMHWVRQAPGKGLEWVSRISSHGSNTYYADSVKGRFTISRDNSKNTLYLQMNSLRAEDTAVYYCARDMYRGVYGFALWGQGTLVTVSS

SEQ ID NO: 53 - DNA, 360 bp, Homo sapiens
gaggtgcaattgctggaaagcggcggcggcctggtgcaaccgggcggcagcctgcgtctgagctgcgcggcctccggatttacctttactacttcttctatgcattgggtgcgccaagcccctgggaagggtctcgagtgggtgagccgtatctcttctcatggtagcaatacctattatgcggatagcgtgaaaggccgttttaccatttcacgtgataattcgaaaaacaccctgtatctgcaaatgaacagcctgcgtgcggaagatacggccgtgtattattgcgcgcgtgatatgtatcgtggtgtttatggttttgctctttggggccaaggcaccctggtgacggttagctca

SEQ ID NO: 54 - protein, 106 aa, Homo sapiens
SYELTQPPSVSVSPGQTASITCSGDAIRNYYVHWYQQKPGQSPVLVIYEDSDRPSGIPERFSGSNSGNTATLTISGTQAMDEADYYCQSYDKSNVVFGGGTKLTVL

SEQ ID NO: 55 - DNA, 318 bp, Homo sapiens
agctacgaactgacccagccgccttcagtgagcgttagcccaggtcagaccgcgagcatcacctgtagcggcgatgctattcgtaattattatgttcattggtaccagcagaaacccgggcagagcccagttcttgtgatttatgaggattctgatcgtccctcaggcatcccggaacgctttagcggatccaacagcggcaacaccgcgaccctgaccattagcggcactcaggcgatggacgaagcggattattattgccagtcttatgataagtctaatgttgtgtttggcggcggcacgaagttaaccgtccta

SEQ ID NO: 56 - protein, 106 aa, Homo sapiens
SYELTQPPSVSVSPGQTASITCSGDAIRNYYVHWYQQKPGQSPKLVIYEDSDRPSGIPERFSGSNSGNTATLTISGTQAMDEADYYCQSYDKSNVVFGGGTKLTVL

SEQ ID NO: 57 - DNA, 318 bp, Homo sapiens
agctacgagctgacccagccccctagcgtgtccgtgagccctggccagaccgccagcatcacatgcagcggcgacgccatccggaactactatgtgcattggtatcagcagaagcccggccagagccccaagctggtcatctacgaggacagcgacagacccagcggcatccccgagagattcagcggcagcaacagcggcaataccgccaccctgaccatcagcggcacccaggccatggacgaggccgactactactgccagagctacgacaagagcaacgtggtgttcggcggagggaccaagctgaccgtccta

SEQ ID NO: 58 - protein, 450 aa, Homo sapiens
EVQLLESGGGLVQPGGSLRLSCAASGFTFSNYVMHWVRQAPGKGLEWVSSIFGSGSDTYYADSVKGRFTISRDNSKNTLYLQMNSLRAEDTAVYYCARSASSGFGFAGYGIDSWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 59 - protein, 213 aa, Homo sapiens
SYELTQPPSVSVSPGQTASITCSGDSLRYYYAHWYQQKPGQSPVLVIYDDNKRPSGIPERFSGSNSGNTATLTISGTQAMDEADYYCQSWDLFHSSVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 60 - DNA, 1350 bp, Homo sapiens
gaggtgcagctgctggagagcggcggcggcctggtgcagcccggcggcagcctgaggctgtcctgcgccgccagcggcttcaccttcagcaactacgtgatgcactgggtgaggcaggcccccggcaagggcctggagtgggtgagcagcatcttcggcagcggcagcgacacctactacgccgacagcgtgaagggcaggttcaccatcagcagggacaacagcaagaacaccctgtacctgcagatgaacagcctgagggccgaggacaccgccgtgtactactgcgccaggagcgccagcagcggcttcggcttcgccggctacggcatcgacagctggggccagggcaccctggtgaccgtgagctcagccagcaccaagggccccagcgtgttccccctggccccctgcagcagaagcaccagcgagagcacagccgccctgggctgcctggtgaaggactacttccccgagcccgtgaccgtgagctggaacagcggagccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagcggaagtgctgcgtggagtgccccccctgccctgcccctcctgtggccggaccctccgtgttcctgttcccccccaagcccaaggacaccctgatgatcagccggacccccgaggtgacctgcgtggtggtggacgtgagccacgaggaccccgaggtgcagtttaattggtacgtggacggcgtggaggtgcacaacgccaagaccaagccccgggaggaacagttcaacagcaccttccgggtggtgtccgtgctgaccgtggtgcaccaggactggctgaacggcaaagaatacaagtgcaaggtgtccaacaagggcctgcctgcccccatcgagaaaaccatcagcaagacaaagggccagcccagggaaccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagccggtggcagcagggcaacgtgttcagctgcagcgtgatgcacgaggccctgcacaaccactacacccagaagagcctgagcctgtcccccggcaaa

SEQ ID NO: 61 - DNA, 639 bp, Homo sapiens
agctacgagctgacccagccccccagcgtgagcgtgagccccggccagaccgccagcatcacctgcagcggcgacagcctgaggtactactacgcccactggtaccagcagaagcccggccagagccccgtgctggtgatctacgacgacaacaagaggcccagcggcatccccgagaggttcagcggcagcaacagcggcaacaccgccaccctgaccatcagcggcacccaggccatggacgaggccgactactactgccagagctgggacctgttccacagcagcgtgttcggcggcggcaccaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 62 - protein, 443 aa, Homo sapiens
EVQLLESGGGLVQPGGSLRLSCAASGFTFNNYAVSWVRQAPGKGLEWVSGISYGGSNTYYADSVKGRFTISRDNSKNTLYLQMNSLRAEDTAVYYCARTLGGDFDHWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 63 - protein, 214 aa, Homo sapiens
QSVLTQPPSVSGAPGQRVTISCSGSSSNIGSNYVNWYQQLPGTAPKLLIYGNSKRPSGVPDRFSGSKSGTSASLAITGLQAEDEADYYCQSFTQMLLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 64 - DNA, 1329 bp, Homo sapiens
gaggtgcaattgctggaaagcggcggcggcctggtgcaaccgggcggcagcctgcgtctgagctgcgcggcctccggatttacctttaataattatgctgtttcttgggtgcgccaagcccctgggaagggtctcgagtgggtgagcggtatctcttatggtggtagcaatacctattatgcggatagcgtgaaaggccgttttaccatttcacgtgataattcgaaaaacaccctgtatctgcaaatgaacagcctgcgtgcggaagatacggccgtgtattattgcgcgcgtactcttggtggtgattttgatcattggggccaaggcaccctggtgacggttagctcagccagcaccaagggccccagcgtgttccccctggccccctgcagcagaagcaccagcgagagcacagccgccctgggctgcctggtgaaggactacttccccgagcccgtgaccgtgagctggaacagcggagccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagcggaagtgctgcgtggagtgccccccctgccctgcccctcctgtggccggaccctccgtgttcctgttcccccccaagcccaaggacaccctgatgatcagccggacccccgaggtgacctgcgtggtggtggacgtgagccacgaggaccccgaggtgcagtttaattggtacgtggacggcgtggaggtgcacaacgccaagaccaagccccgggaggaacagttcaacagcaccttccgggtggtgtccgtgctgaccgtggtgcaccaggactggctgaacggcaaagaatacaagtgcaaggtgtccaacaagggcctgcctgcccccatcgagaaaaccatcagcaagacaaagggccagcccagggaaccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagccggtggcagcagggcaacgtgttcagctgcagcgtgatgcacgaggccctgcacaaccactacacccagaagagcctgagcctgtcccccggcaaa

SEQ ID NO: 65 - DNA, 642 bp, Homo sapiens
cagagcgtgctgacccagccgccttcagtgagtggcgcaccaggtcagcgtgtgaccatctcgtgtagcggcagcagcagcaacattggttctaattatgtgaattggtaccagcagttgcccgggacggcgccgaaacttctgatttatggtaattctaagcgtccctcaggcgtgccggatcgttttagcggatccaaaagcggcaccagcgcgagccttgcgattacgggcctgcaagccgaagacgaagcggattattattgccagtcttttactcagatgcttcttgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 66 - protein, 446 aa, Homo sapiens
EVQLLESGGGLVQPGGSLRLSCAASGFTFTTSSMHWVRQAPGKGLEWVSRISSHGSNTYYADSVKGRFTISRDNSKNTLYLQMNSLRAEDTAVYYCARDMYRGVYGFALWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - protein, 212 aa, Homo sapiens
SYELTQPPSVSVSPGQTASITCSGDAIRNYYVHWYQQKPGQSPVLVIYEDSDRPSGIPERFSGSNSGNTATLTISGTQAMDEADYYCQSYDKSNVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 68 - DNA, 1338 bp, Homo sapiens
gaggtgcaattgctggaaagcggcggcggcctggtgcaaccgggcggcagcctgcgtctgagctgcgcggcctccggatttacctttactacttcttctatgcattgggtgcgccaagcccctgggaagggtctcgagtgggtgagccgtatctcttctcatggtagcaatacctattatgcggatagcgtgaaaggccgttttaccatttcacgtgataattcgaaaaacaccctgtatctgcaaatgaacagcctgcgtgcggaagatacggccgtgtattattgcgcgcgtgatatgtatcgtggtgtttatggttttgctctttggggccaaggcaccctggtgacggttagctcagccagcaccaagggccccagcgtgttccccctggccccctgcagcagaagcaccagcgagagcacagccgccctgggctgcctggtgaaggactacttccccgagcccgtgaccgtgagctggaacagcggagccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagcggaagtgctgcgtggagtgccccccctgccctgcccctcctgtggccggaccctccgtgttcctgttcccccccaagcccaaggacaccctgatgatcagccggacccccgaggtgacctgcgtggtggtggacgtgagccacgaggaccccgaggtgcagtttaattggtacgtggacggcgtggaggtgcacaacgccaagaccaagccccgggaggaacagttcaacagcaccttccgggtggtgtccgtgctgaccgtggtgcaccaggactggctgaacggcaaagaatacaagtgcaaggtgtccaacaagggcctgcctgcccccatcgagaaaaccatcagcaagacaaagggccagcccagggaaccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagccggtggcagcagggcaacgtgttcagctgcagcgtgatgcacgaggccctgcacaaccactacacccagaagagcctgagcctgtcccccggcaaa

SEQ ID NO: 69 - DNA, 636 bp, Homo sapiens
agctacgaactgacccagccgccttcagtgagcgttagcccaggtcagaccgcgagcatcacctgtagcggcgatgctattcgtaattattatgttcattggtaccagcagaaacccgggcagagcccagttcttgtgatttatgaggattctgatcgtccctcaggcatcccggaacgctttagcggatccaacagcggcaacaccgcgaccctgaccattagcggcactcaggcgatggacgaagcggattattattgccagtcttatgataagtctaatgttgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 70 - DNA, 23 bp, Artificial Sequence
  Primer
cctaccgttcgtcttcacccctg

SEQ ID NO: 71 - DNA, 21 bp, Artificial Sequence
  Primer
ggcactggctggtttcgctac

SEQ ID NO: 72 - DNA, 20 bp, Artificial Sequence
  Primer
ctcggagccagcggaaacac

SEQ ID NO: 73 - DNA, 22 bp, Artificial Sequence
  Primer
cggaaaaataaacacgctcgga

SEQ ID NO: 74 - DNA, 22 bp, Artificial Sequence
  Primer
gctcacactcggtgcggctttc

SEQ ID NO: 75 - protein, 10 aa, Homo sapiens
QAWDLINSHV

SEQ ID NO: 76 - protein, 107 aa, Homo sapiens
DIELTQPPSVSVAPGQTARISCSGDSLRYYYAHWYQQKPGQAPVLVIYDDNKRPSGIPERFSGSNSGNTATLTISGTQAEDEADYYCQAWDLINSHVFGGGTKLTVL

SEQ ID NO: 77 - DNA, 321 bp, Homo sapiens
gatatcgaactgacccagccgccttcagtgagcgttgcaccaggtcagaccgcgcgtatctcgtgtagcggcgattctcttcgttattattatgctcattggtaccagcagaaacccgggcaggcgccagttcttgtgatttatgatgataataagcgtccctcaggcatcccggaacgctttagcggatccaacagcggcaacaccgcgaccctgaccattagcggcactcaggcggaagacgaagcggattattattgccaggcttgggatcttattaattctcatgtgtttggcggcggcacgaagttaaccgtccta

SEQ ID NO: 78 - protein, 213 aa, Homo sapiens
DIELTQPPSVSVAPGQTARISCSGDSLRYYYAHWYQQKPGQAPVLVIYDDNKRPSGIPERFSGSNSGNTATLTISGTQAEDEADYYCQAWDLINSHVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS